SEQUENCE LISTING (18 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 3 - protein, 222 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRPSQGSHLPSCLSKEPLGRRNPLPTHFHPSPPGLHLSHQDSSSQRPLGCSLAWTEARDTSTQ

SEQ ID NO: 4 - DNA, 1044 bp, Homo sapiens
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 5 - DNA, 860 bp, Homo sapiens
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 6 - DNA, 1012 bp, Homo sapiens
ccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggata

SEQ ID NO: 7 - DNA, 696 bp, Artificial Sequence
  Synthetic Construct
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgatga

SEQ ID NO: 8 - DNA, 696 bp, Artificial Sequence
  Synthetic Construct
atggagcctctgagactgctgattctgctgtttgtcactgaactgagcggcgcacataataccactgtcttccagggcgtcgctgggcagtctctgcaggtgagctgcccctacgactctatgaagcactggggccggagaaaggcatggtgccggcagctgggagagaagggaccttgtcagagagtggtgagcacccacaacctgtggctgctgtccttcctgaggcgctggaatggctctacagccatcaccgacgatacactgggcggcaccctgacaatcaccctgaggaacctgcagcctcacgacgcaggcctgtatcagtgccagtccctgcacggctctgaggccgatacactgaggaaggtgctggtggaggtgctggccgaccctctggatcacagggacgcaggcgatctgtggttcccaggcgagagcgagtcctttgaggatgcccacgtggagcactctatcagccggtccctgctggagggagagatcccattcccccctaccagcatcctgctgctgctggcctgtatctttctgatcaagatcctggcagcatccgccctgtgggcagcagcctggcacggacagaagccaggaacacacccaccatccgagctggattgcggacatgaccccggctaccagctgcagacactgcctggcctgagggatacatgatga

SEQ ID NO: 9 - DNA, 663 bp, Artificial Sequence
  Synthetic Construct
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgatga

SEQ ID NO: 10 - DNA, 663 bp, Artificial Sequence
  Synthetic Construct
atggagcctctgcggctgctgatcctgctgttcgtgaccgagctgtccggcgcccacaacaccacagtgtttcagggagtggcaggacagtccctgcaggtgtcttgcccatacgactctatgaagcactggggccggagaaaggcatggtgcaggcagctgggagagaagggaccatgtcagcgcgtggtgtctacacacaacctgtggctgctgagcttcctgaggcgctggaatggctccacagccatcaccgacgatacactgggcggcaccctgacaatcaccctgaggaatctgcagccacacgacgccggcctgtatcagtgtcagagcctgcacggctccgaggcagataccctgcggaaggtgctggtggaggtgctggccgaccccctggatcacagagacgcaggcgatctgtggttccctggcgagagcgagtcctttgaggatgcccacgtggagcactctatcagccgggccgagagacacgtgaaggaggacgatggaaggaagtctcctggagaggtgccacctggaaccagcccagcatgcatcctggcaacatggccaccaggcctgctggtgctgctgtggcaggagacaacactgcccgagcactgtttttcctggaccctggaggccggcacaggctgatga

SEQ ID NO: 11 - DNA, 1945 bp, Artificial Sequence
  Synthetic Construct
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgatgattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgatga

SEQ ID NO: 12 - DNA, 1945 bp, Artificial Sequence
  Synthetic Construct
atggagcctctgagactgctgattctgctgtttgtcactgaactgagcggcgcacataataccactgtcttccagggcgtcgctgggcagtctctgcaggtgagctgcccctacgactctatgaagcactggggccggagaaaggcatggtgccggcagctgggagagaagggaccttgtcagagagtggtgagcacccacaacctgtggctgctgtccttcctgaggcgctggaatggctctacagccatcaccgacgatacactgggcggcaccctgacaatcaccctgaggaacctgcagcctcacgacgcaggcctgtatcagtgccagtccctgcacggctctgaggccgatacactgaggaaggtgctggtggaggtgctggccgaccctctggatcacagggacgcaggcgatctgtggttcccaggcgagagcgagtcctttgaggatgcccacgtggagcactctatcagccggtccctgctggagggagagatcccattcccccctaccagcatcctgctgctgctggcctgtatctttctgatcaagatcctggcagcatccgccctgtgggcagcagcctggcacggacagaagccaggaacacacccaccatccgagctggattgcggacatgaccccggctaccagctgcagacactgcctggcctgagggatacatgatgattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatggagcctctgcggctgctgatcctgctgttcgtgaccgagctgtccggcgcccacaacaccacagtgtttcagggagtggcaggacagtccctgcaggtgtcttgcccatacgactctatgaagcactggggccggagaaaggcatggtgcaggcagctgggagagaagggaccatgtcagcgcgtggtgtctacacacaacctgtggctgctgagcttcctgaggcgctggaatggctccacagccatcaccgacgatacactgggcggcaccctgacaatcaccctgaggaatctgcagccacacgacgccggcctgtatcagtgtcagagcctgcacggctccgaggcagataccctgcggaaggtgctggtggaggtgctggccgaccccctggatcacagagacgcaggcgatctgtggttccctggcgagagcgagtcctttgaggatgcccacgtggagcactctatcagccgggccgagagacacgtgaaggaggacgatggaaggaagtctcctggagaggtgccacctggaaccagcccagcatgcatcctggcaacatggccaccaggcctgctggtgctgctgtggcaggagacaacactgcccgagcactgtttttcctggaccctggaggccggcacaggctgatga

SEQ ID NO: 13 - protein, 317 aa, Artificial Sequence [sequence omitted]

SEQ ID NO: 14 - DNA, 385 bp, Artificial Sequence
  Synthetic Construct
gatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgg

SEQ ID NO: 15 - DNA, 6821 bp, Artificial Sequence
  Synthetic Construct
cgcgttggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgggcccgacgtcgcatgctccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagctagcggtaccgatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggatccaccggtgccaccatggagcctctgcggctgctgatcctgctgttcgtgaccgagctgtccggcgcccacaacaccacagtgtttcagggagtggcaggacagtccctgcaggtgtcttgcccatacgactctatgaagcactggggccggagaaaggcatggtgcaggcagctgggagagaagggaccatgtcagcgcgtggtgtctacacacaacctgtggctgctgagcttcctgaggcgctggaatggctccacagccatcaccgacgatacactgggcggcaccctgacaatcaccctgaggaatctgcagccacacgacgccggcctgtatcagtgtcagagcctgcacggctccgaggcagataccctgcggaaggtgctggtggaggtgctggccgaccccctggatcacagagacgcaggcgatctgtggttccctggcgagagcgagtcctttgaggatgcccacgtggagcactctatcagccgggccgagagacacgtgaaggaggacgatggaaggaagtctcctggagaggtgccacctggaaccagcccagcatgcatcctggcaacatggccaccaggcctgctggtgctgctgtggcaggagacaacactgcccgagcactgtttttcctggaccctggaggccggcacaggctgatgagtcgacgaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagagga

SEQ ID NO: 16 - DNA, 398 bp, Artificial Sequence
  Synthetic Construct
ttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 17 - DNA, 483 bp, Artificial Sequence
  Synthetic Construct
atttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaacccctggttcacctccttccaggttctgg

SEQ ID NO: 18 - DNA, 1191 bp, Artificial Sequence
  Synthetic Construct
atttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaacccctggttcacctccttccaggttctggggatccaccggtatggagcctctgagactgctgattctgctgtttgtcactgaactgagcggcgcacataataccactgtcttccagggcgtcgctgggcagtctctgcaggtgagctgcccctacgactctatgaagcactggggccggagaaaggcatggtgccggcagctgggagagaagggaccttgtcagagagtggtgagcacccacaacctgtggctgctgtccttcctgaggcgctggaatggctctacagccatcaccgacgatacactgggcggcaccctgacaatcaccctgaggaacctgcagcctcacgacgcaggcctgtatcagtgccagtccctgcacggctctgaggccgatacactgaggaaggtgctggtggaggtgctggccgaccctctggatcacagggacgcaggcgatctgtggttcccaggcgagagcgagtcctttgaggatgcccacgtggagcactctatcagccggtccctgctggagggagagatcccattcccccctaccagcatcctgctgctgctggcctgtatctttctgatcaagatcctggcagcatccgccctgtgggcagcagcctggcacggacagaagccaggaacacacccaccatccgagctggattgcggacatgaccccggctaccagctgcagacactgcctggcctgagggatacatgatga

(19) United States
(12) Patent Application Publication
(10) Pub. No.: US 2023/0322897 A1
(43) Pub. Date: Oct. 12, 2023

(54) COMPOSITIONS AND METHODS FOR TREATING NEUROCOGNITIVE DISORDERS

(71) Applicant: AvroBio, Inc., Cambridge, MA (US)

(72) Inventors: Chris MASON, Cambridge, MA (US); Oliver COOPER, Jamaica Plain, MA (US); Robert PLASSCHAERT, Cambridge, MA (US)

(21) Appl. No.: 18/014,311

(22) PCT Filed: Jun. 29, 2021
(86) PCT No.: PCT/US2021/039613

§ 371 Date: Jan. 3, 2023

Related U.S. Application Data
(60) Provisional application No. 63/047,744, filed on Jul. 2, 2020.

(51) Int. Cl. C07K 14/705; A61P 25/28; A61P 25/00; A61P 25/16; A61K 35/28; A61K 35/545; C07K 14/47; A61K 31/7088; A61K 38/46

(52) U.S. Cl. CPC C07K 14/70503; A61P 25/28; A61P 25/00; A61P 25/16; A61K 35/28; A61K 35/545; C07K 14/47; A61K 31/7088; A61K 38/465; C07K 2319/00

(57) ABSTRACT

Described herein are compositions and methods for treating a subject having or at risk of developing a neurocognitive disorder, such as Alzheimer's disease or Nasu-Hakola disease. For example, using the compositions and methods of the disclosure, a subject having or at risk of developing a neurocognitive disorder may be administered one or more cells that contain a transgene encoding triggering receptor expressed on myeloid cells two (TREM2), such as a population of CD34+ hematopoietic stem or progenitor cells that express TREM2, thereby treating or preventing the disorder.

### SEQUENCE LISTING

The instant application contains a Sequence Listing which has been submitted electronically in ASCII format and is hereby incorporated by reference in its entirety. Said ASCII copy, created on Jun. 28, 2021, is named “51182-027WO2_Sequence_Listing_6_28_21_ST25” and is 35,497 bytes in size.

### FIELD OF THE DISCLOSURE

The disclosure relates to compositions and methods for treating neurocognitive disorders, such as Alzheimer's disease, Nasu-Hakola Disease, frontotemporal lobar degeneration, and Parkinson disease.

### BACKGROUND

Neurodegeneration is a pathophysiological process that is observed in a number of diseases associated with progressive dementia, such as Alzheimer's disease and Nasu-Hakola Disease. A key feature of this process is the neuronal degeneration and death that causes the wholesale destruction of brain tissue and the accompanying gamut of behavioral deficits including cognitive decline, language impairments, among others.

Alzheimer's disease (AD) is a late-onset neurodegenerative disorder responsible for the majority of dementia cases in the elderly. AD patients suffer from a progressive cognitive decline characterized by symptoms including an insidious loss of short- and long-term memory, attention deficits, language-specific problems, disorientation, impulse control, social withdrawal, anhedonia, and other symptoms. Distinguishing neuropathological features of AD are extracellular aggregates of amyloid-3 plaques and neurofibrillary tangles composed of hyperphosphorylated microtubule-associated tau proteins. Accumulation of these aggregates is associated with neuronal loss and atrophy in a number of brain regions including the frontal, temporal, and parietal lobes of the cerebral cortex as well as subcortical structures like the basal forebrain cholinergic system and the locus coeruleus within the brainstem. AD is also associated with increased neuroinflammation characterized by reactive gliosis and elevated levels of pro-inflammatory cytokines.

Nasu-Hakola Disease, also known as polycystic lipomembranous osteodysplasia with sclerosing leukoencephalopathy (PLOSL) is a neurodegenerative disorder characterized by the presence of white matter degeneration, axonal spheroids, as well as cystic bone lesions in the upper and lower extremities. PLOSL patients exhibit early onset dementia as well as recurrent bone fractures. Unlike AD, which largely affects older patients, PLOSL may begin manifesting during adolescence during the osseous stage when patients may experience polyarthralgia in hands, wrists, ankles, and feet. The osseous stage is followed by the early neurological stage during which patients may exhibit profound personality changes, progressive memory deficits, and epileptic seizures. The late neurological stage of PLOSL patients presents with profound dementia and motor incapacitation.

Parkinson's disease (PD) is a progressive disorder of the nervous system that affects movement and produces symptoms such as resting tremor, rigidity, and bradykinesia. Patients suffering from Parkinson's disease may also experience non-motor symptoms, including depression, constipation, pain, sleep disorders, cognitive decline, and olfactory dysfunction. Post-mortem analyses of PD patient brains often reveal Lewy bodies containing α-synuclein in affected brain regions and a loss of dopaminergic neurons in the substantia nigra pars compacta.

Frontotemporal lobar degeneration (FTLD) is a neurodegenerative disorder characterized by a complex clinical presentation that may include deficits in speech comprehension and production, poor motor planning and coordination, and/or loss of executive function characterized by lack of impulse control and a preference for perseverative behaviors. Clinical descriptions of FTLD separate into three distinct variants: 1) behavioral-frontotemporal dementia, characterized by profound changes in behavior, personality, and pronounced degeneration of the frontal lobe; 2) semantic dementia, characterized by insidious degradation of language and pronounced degeneration in the anterior temporal lobe; and 3) progressive nonfluent aphasia, characterized by deficits in speech and grammar and corresponding to degeneration of left perisylvian cortex. Histological analyses of post-mortem brain tissue from FTLD patients exhibit complex and heterogeneous neuropathological profiles with the common presentation of degeneration of neural tissue in the frontal and temporal lobes of the brain.

Current treatments for the above indications strive to ameliorate disease symptomology, but therapies targeting the underlying neurodegeneration are lacking, thus underscoring the need for new therapeutic avenues.

### SUMMARY OF THE DISCLOSURE

The present disclosure provides methods for treating a neurocognitive disorder (NCD; e.g., Alzheimer's disease (AD), Nasu-Hakola Disease (also known as polycystic lipomembranous osteodysplasia with sclerosing leukoencephalopathy; PLOSL), frontotemporal lobar degeneration (FTLD), and Parkinson disease (PD)) by administering cells, such as pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia containing a transgene encoding TREM2 (“triggering receptor expressed on myeloid cells two”). The cells may be administered to a subject (e.g., a human) having an NCD by one or more of a variety of routes, including directly to the central nervous system of the subject (e.g., by intracerebroventricular administration) or systemically (e.g., by intravenous administration), among others. The disclosure also features compositions containing such cells, as well as kits containing these cells for the treatment of an NCD.

In a first aspect, the disclosure provides a method of treating a subject diagnosed as having an NCD (e.g., AD, PLOSL, FTLD, or PD) by administering to the subject a composition containing a population of cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding TREM2 operably linked to a high expression constitutive promoter. In some embodiments, the high expression constitutive promoter is an MND promoter.

In some embodiments, the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 14. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 14. In some embodiments, the MND promoter includes a polynucleotide having at least 95% (e.g., at least 96%, 97%, 98%, 99%, or more) sequence identity to the nucleic acid sequence of SEQ ID NO. 14. In some embodiments, the MND promoter includes a polynucleotide having at least 98% (e.g., at least 99% or more) sequence identity to the nucleic acid sequence of SEQ ID NO. 14. In some embodiments, the MND promoter includes a polynucleotide having at least 99% sequence identity to the nucleic acid sequence of SEQ ID NO. 14. In some embodiments, the MND promoter includes a polynucleotide having the nucleic acid sequence of SEQ ID NO. 14.

In some embodiments, the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 16. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 16. In some embodiments, the MND promoter includes a polynucleotide having at least 95% (e.g., at least 96%, 97%, 98%, 99%, or more) sequence identity to the nucleic acid sequence of SEQ ID NO. 16. In some embodiments, the MND promoter includes a polynucleotide having at least 98% (e.g., at least 99% or more) sequence identity to the nucleic acid sequence of SEQ ID NO. 16. In some embodiments, the MND promoter includes a polynucleotide having at least 99% sequence identity to the nucleic acid sequence of SEQ ID NO. 16. In some embodiments, the MND promoter includes a polynucleotide having the nucleic acid sequence of SEQ ID NO. 16.

In some embodiments, the high expression constitutive promoter is an integrin subunit alpha M (CD11b) promoter. In some embodiments, the CD11b promoter comprises a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 17, optionally wherein the CD11b promoter comprises a polynucleotide having at least 90% % (e.g., at least 91%, 92%, 93%, 94%, 95%, 96%, 97%, 98%, 99%, or more) sequence identity to the nucleic acid sequence of SEQ ID NO. 17, optionally wherein the CD11b promoter comprises a polynucleotide having at least 95% (e.g., at least 96%, 97%, 98%, 99%, or more) sequence identity to the nucleic acid sequence of SEQ ID NO. 17, optionally wherein the CD11b promoter comprises a polynucleotide having the nucleic acid sequence of SEQ ID NO. 17. In some embodiments, the CD11b promoter includes a polynucleotide having at least 98% (e.g., at least 99% or more) sequence identity to the nucleic acid sequence of SEQ ID NO. 17. In some embodiments, the CD11b promoter includes a polynucleotide having at least 99% sequence identity to the nucleic acid sequence of SEQ ID NO. 17. In some embodiments, the CD11b promoter includes a polynucleotide having the nucleic acid sequence of SEQ ID NO. 17.

In some embodiments, the transgene encoding TREM2 is capable of expression in a macrophage or a microglial cell. In some embodiments, the cell expresses the transgene encoding TREM2.

In some embodiments, the NCD is a major NCD. In some embodiments, the major NCD interferes with the subject's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the subject on a cognitive test that is at least two standard deviations away from the mean score of a reference population. In some embodiments, the NCD is a mild NCD. In some embodiments, the mild NCD does not interfere with the subject's independence and/or normal daily functioning. In some embodiments, the mild NCD is associated with a score obtained by the subject on a cognitive test that is between one to two standard deviations away from the mean score of a reference population. In some embodiments, the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the subject's age, medical history, education, socioeconomic status, and lifestyle. In some embodiments, the NCD is AD. In some embodiments, the NCD is a leukodystrophy. In some embodiments, the NCD is PLOSL. In some embodiments, the NCD is a frontotemporal NCD. In some embodiments the frontotemporal NCD is a FTLD. In some embodiments, the FTLD is the behavioral-variant frontotemporal dementia (BVFTD). In some embodiments, the FTLD is the semantic dementia (SD) variant of FTLD. In some embodiments, the FTLD is the progressive nonfluent aphasia (PNA) variant of FTLD. In some embodiments, the NCD is a movement disorder. In some embodiments, the movement disorder is PD.

In some embodiments, the TREM2 is full-length TREM2, such as TREM2 having an amino acid sequence of any one of SEQ ID NOS. 1-3 or a variant thereof having at least 85% sequence identity thereto (e.g. at least 86%, 90%, 95%, 96%, 97%, 98%, 99%, or more) sequence identity to the amino acid sequence of any one of SEQ ID NOS. 1-3.

In some embodiments, the TREM2 has an amino acid sequence of SEQ ID NO. 1 or is a variant thereof having at least 85% (e.g. at least 86%, 90%, 95%, 96%, 97%, 98%, 99%, or more) sequence identity to the amino acid sequence of SEQ ID NO. 1. In some embodiments, the TREM2 has an amino acid sequence having at least 90% (e.g., at least 91%, 92%, 93%, 94%, 95%, 96%, 97%, 98%, 99%, or more) sequence identity to the amino acid sequence of SEQ ID NO. 1. In some embodiments, the TREM2 has an amino acid sequence having at least 95% (e.g., at least 96%, 97%, 98%, 99%, or more) sequence identity to the amino acid sequence of SEQ ID NO. 1. In some embodiments, the TREM2 has an amino acid sequence having at least 98% (e.g., at least 99% or more) sequence identity to the amino acid sequence of SEQ ID NO. 1. In some embodiments, the TREM2 has an amino acid sequence having at least 99% sequence identity to the amino acid sequence of SEQ ID NO. 1. In some embodiments, the TREM2 has the amino acid sequence of SEQ ID NO. 1.

In some embodiments, the TREM2 has an amino acid sequence of SEQ ID NO. 2 or is a variant thereof having at least 85% (e.g. at least 86%, 90%, 95%, 96%, 97%, 98%, 99%, or more) sequence identity to the amino acid sequence of SEQ ID NO. 2. In some embodiments, the TREM2 has an amino acid sequence having at least 90% (e.g., at least 91%, 92%, 93%, 94%, 95%, 96%, 97%, 98%, 99%, or more) sequence identity to the amino acid sequence of SEQ ID NO. 2. In some embodiments, the TREM2 has an amino acid sequence having at least 95% (e.g., at least 96%, 97%, 98%, 99%, or more) sequence identity to the amino acid sequence of SEQ ID NO. 2. In some embodiments, the TREM2 has an amino acid sequence having at least 98% (e.g., at least 99% or more) sequence identity to the amino acid sequence of SEQ ID NO. 2. In some embodiments, the TREM2 has an amino acid sequence having at least 99% sequence identity to the amino acid sequence of SEQ ID NO. 2. In some embodiments, the TREM2 has the amino acid sequence of SEQ ID NO. 2.

In some embodiments, the TREM2 has an amino acid sequence of SEQ ID NO. 3 or is a variant thereof having at least 85% (e.g. at least 86%, 90%, 95%, 96%, 97%, 98%, 99%, or more) sequence identity to the amino acid sequence of SEQ ID NO. 3. In some embodiments, the TREM2 has an amino acid sequence having at least 90% (e.g., at least 91%, 92%, 93%, 94%, 95%, 96%, 97%, 98%, 99%, or more) sequence identity to the amino acid sequence of SEQ ID NO. 3. In some embodiments, the TREM2 has an amino acid sequence having at least 95% (e.g., at least 96%, 97%, 98%, 99%, or more) sequence identity to the amino acid sequence of SEQ ID NO. 3. In some embodiments, the TREM2 has an amino acid sequence having at least 98% (e.g., at least 99% or more) sequence identity to the amino acid sequence of SEQ ID NO. 3. In some embodiments, the TREM2 has an amino acid sequence having at least 99% sequence identity to the amino acid sequence of SEQ ID NO. 3. In some embodiments, the TREM2 has the amino acid sequence of SEQ ID NO. 3.

In some embodiments, the transgene encoding TREM2 includes a TREM2 polynucleotide having the nucleic acid sequence of SEQ ID NO. 4 or a variant thereof having at least 85% (e.g. at least 86%, 90%, 95%, 96%, 97%, 98%, 99%, or more) sequence identity to the nucleic acid sequence of SEQ ID NO. 4. In some embodiments, the transgene encoding TREM2 includes a TREM2 polynucleotide having at least 90% (e.g., at least 91%, 92%, 93%, 94%, 95%, 96%, 97%, 98%, 99%, or more) sequence identity to the nucleic acid sequence of SEQ ID NO. 4. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 4. In some embodiments, the transgene encoding TREM2 includes a TREM2 polynucleotide having at least 98% (e.g., at least 99% or more) sequence identity to the nucleic acid sequence of SEQ ID NO. 4. In some embodiments, the transgene encoding TREM2 includes a TREM2 polynucleotide having at least 99% sequence identity to the nucleic acid sequence of SEQ ID NO. 4. In some embodiments, the transgene encoding TREM2 includes a TREM2 polynucleotide having the nucleic acid sequence of SEQ ID NO. 4.

In some embodiments, the transgene encoding TREM2 includes a TREM2 polynucleotide having the nucleic acid sequence of SEQ ID NO. 5 or a variant thereof having at least 85% (e.g. at least 86%, 90%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 5. In some embodiments, the transgene encoding TREM2 includes a TREM2 polynucleotide having at least 98% (e.g. at least 99% or more) sequence identity to the nucleic acid sequence of SEQ ID NO. 5. In some embodiments, the transgene encoding TREM2 includes a TREM2 polynucleotide having at least 99% sequence identity to the nucleic acid sequence of SEQ ID NO. 5. In some embodiments, the transgene encoding TREM2 includes a TREM2 polynucleotide having the nucleic acid sequence of SEQ ID NO. 5.

In some embodiments, the transgene encoding TREM2 includes a TREM2 polynucleotide having the nucleic acid sequence of SEQ ID NO. 6 or a variant thereof having at least 85% (e.g. at least 86%, 90%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 6. In some embodiments, the transgene encoding TREM2 includes a TREM2 polynucleotide having at least 98% (e.g. at least 99% or more) sequence identity to the nucleic acid sequence of SEQ ID NO. 6. In some embodiments, the transgene encoding TREM2 includes a TREM2 polynucleotide having at least 99% sequence identity to the nucleic acid sequence of SEQ ID NO. 6. In some embodiments, the transgene encoding TREM2 includes a TREM2 polynucleotide having the nucleic acid sequence of SEQ ID NO. 6.

In some embodiments, the transgene encoding TREM2 includes a TREM2 polynucleotide having the nucleic acid sequence of SEQ ID NO. 7 or a variant thereof having at least 85% (e.g. at least 86%, 90%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 7. In some embodiments, the transgene encoding TREM2 includes a TREM2 polynucleotide having at least 98% (e.g. at least 99% or more) sequence identity to the nucleic acid sequence of SEQ ID NO. 7. In some embodiments, the transgene encoding TREM2 includes a TREM2 polynucleotide having at least 99% sequence identity to the nucleic acid sequence of SEQ ID NO. 7. In some embodiments, the transgene encoding TREM2 includes a TREM2 polynucleotide having the nucleic acid sequence of SEQ ID NO. 7.

In some embodiments, the transgene encoding TREM2 includes a TREM2 polynucleotide having the nucleic acid sequence of SEQ ID NO. 9 or a variant thereof having at least 85% (e.g. at least 86%, 90%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 9. In some embodiments, the transgene encoding TREM2 includes a TREM2 polynucleotide having at least 98% (e.g. at least 99% or more) sequence identity to the nucleic acid sequence of SEQ ID NO. 9. In some embodiments, the transgene encoding TREM2 includes a TREM2 polynucleotide having at least 99% sequence identity to the nucleic acid sequence of SEQ ID NO. 9. In some embodiments, the transgene encoding TREM2 includes a TREM2 polynucleotide having the nucleic acid sequence of SEQ ID NO. 9.

In some embodiments, the transgene encoding TREM2 includes a TREM2 polynucleotide having the nucleic acid sequence of SEQ ID NO. 11 or a variant thereof having at least 85% (e.g. at least 86%, 90%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 11. In some embodiments, the transgene encoding TREM2 includes a TREM2 polynucleotide having at least 98% (e.g. at least 99% or more) sequence identity to the nucleic acid sequence of SEQ ID NO. 11. In some embodiments, the transgene encoding TREM2 includes a TREM2 polynucleotide having at least 99% sequence identity to the nucleic acid sequence of SEQ ID NO. 11. In some embodiments, the transgene encoding TREM2 includes a TREM2 polynucleotide having the nucleic acid sequence of SEQ ID NO. 11.

In some embodiments, the transgene encoding TREM2 may be codon-optimized (e.g., any one of SEQ ID NO. 8, SEQ ID NO. 10, or SEQ ID NO. 12).

In some embodiments, the transgene encoding TREM2 includes a codon-optimized TREM2 polynucleotide of SEQ ID NO. 8 or a variant thereof having at least 85% (e.g. at least 86%, 90%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 98% (e.g. at least 99% or more) sequence identity to the nucleic acid sequence of SEQ ID NO. 8. In some embodiments, the transgene encoding TREM2 includes a codon-optimized TREM2 polynucleotide having at least 99% sequence identity to the nucleic acid sequence of SEQ ID NO. 8. In some embodiments, the transgene encoding TREM2 includes a codon-optimized TREM2 polynucleotide of SEQ ID NO. 8.

In some embodiments, the transgene encoding TREM2 includes a codon-optimized TREM2 polynucleotide of SEQ ID NO. 10 or a variant thereof having at least 85% (e.g. at least 86%, 90%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 98% (e.g. at least 99% or more) sequence identity to the nucleic acid sequence of SEQ ID NO. 10. In some embodiments, the transgene encoding TREM2 includes a codon-optimized TREM2 polynucleotide having at least 99% sequence identity to the nucleic acid sequence of SEQ ID NO. 10. In some embodiments, the transgene encoding TREM2 includes a codon-optimized TREM2 polynucleotide of SEQ ID NO. 10.

In some embodiments, the transgene encoding TREM2 includes a codon-optimized TREM2 polynucleotide of SEQ ID NO. 12 or a variant thereof having at least 85% (e.g. at least 86%, 90%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 98% (e.g. at least 99% or more) sequence identity to the nucleic acid sequence of SEQ ID NO. 12. In some embodiments, the transgene encoding TREM2 includes a codon-optimized TREM2 polynucleotide having at least 99% sequence identity to the nucleic acid sequence of SEQ ID NO. 12. In some embodiments, the transgene encoding TREM2 includes a codon-optimized TREM2 polynucleotide of SEQ ID NO. 12.

In some embodiments, the transgene encodes two or more TREM2 proteins (e.g., at least 2, 3, 4, 5, 6, 7, 8, 9, 10 or more TREM2 proteins). In some embodiments, the transgene encodes from two to ten TREM2 proteins (e.g., 2, 3, 4, 5, 6, 7, 8, 9, or 10 TREM2 proteins). In some embodiments, the transgene encodes from two to five TREM2 proteins (e.g., 2, 3, 4, or 5 TREM2 proteins). In some embodiments, the transgene encodes two TREM2 proteins. In some embodiments, the TREM2 transgenes are expressed from a single, polycistronic expression cassette. In some embodiments, the TREM2 transgenes are separated from one another by way of one or more (e.g., 1, 2, 3, 4, 5, 6, 7, 8, 9, or more) internal ribosome entry sites (IRES). In some embodiments, the TREM2 transgenes are expressed from one or more (e.g., 1, 2, 3, 4, 5, 6, 7, 8, 9, 10, or more) monocistronic expression cassettes.

In some embodiments, the transgene encoding TREM2 includes a signal peptide (e.g., a TREM2 signal peptide).

In some embodiments, the TREM2 is soluble TREM2 (sTREM2). In some embodiments, the TREM2 is the TREM2 C-terminal fragment (TREM2-CTF). In some embodiments, the TREM2 is the TREM2 intracellular domain (TREM2-ICD). In some embodiments, the TREM2 is the TREM2-A p-like (TREM-T23) peptide. In some embodiments, the TREM2 lacks a functional ectodomain cleavage site. In some embodiments, the TREM2 lacks a functional intramembrane cleavage site within the TREM2-CTF.

In some embodiments, the TREM2 is a TREM2 fusion protein. In some embodiments, the TREM2 fusion protein contains a low-density lipoprotein receptor family (LDLRf) binding (Rb) domain of apolipoprotein E (ApoE), or a fragment, variant, or oligomer thereof. In some embodiments, the Rb domain of ApoE, or a fragment, variant, or oligomer thereof, is operably linked to the N-terminus of the TREM2. In some embodiments, the Rb domain of ApoE, or a fragment, variant, or oligomer thereof is operably linked to the C-terminus of the TREM2. In some embodiments, the TREM2 fusion protein contains 1 or more (e.g., 1, 2, 3, 4, 5, 6, 7, 8, 9, 10, or more) oligomers of the Rb domain of ApoE. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 25-185 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 50-180 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 75-175 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 100-170 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 125-160 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 130-150 of SEQ ID NO. 13. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 148-173 or a portion thereof containing residues 159-167 of SEQ ID NO. 13, or a variant having at least 70% sequence identity (e.g., at least 70%, 75%, 80%, 85%, 90%, 95%, 96%, 97%, 98%, 99%, or greater, sequence identity) to residues 159-167 of SEQ ID NO. 13. In some embodiments, the Rb domain contains a region having at least 70% sequence identity (e.g., at least 70%, 75%, 80%, 85%, 90%, 95%, 96%, 97%, 98%, 99%, or greater, sequence identity) to the amino acid sequence of residues 159-167 of SEQ ID NO. 13.

In some embodiments, the transgene encoding TREM2 further contains a microRNA (miRNA) targeting sequence (e.g., a miR-126 targeting sequence). In some embodiments, the miRNA targeting sequence (e.g., a miR-126 targeting sequence) is located within the 3′-untranslated region (UTR) of the transgene.

In some embodiments, the TREM2 penetrates the blood brain barrier (BBB) in the subject.

In some embodiments, the NCD is TREM2-associated NCD. In some embodiments, the AD or PLOSL is TREM2-associated AD, PLOSL, FTLD, or PD.

In some embodiments, the subject suffering from TREM2-associated AD or PLOSL carries a mutation in the TREM2 gene. The mutation in the TREM2 gene may result in an amino acid substitution (e.g., p.R47H, p.R62H, p.T66M, p.T66M, p.Y38C, p.T96K, p.D87N, p.H157Y, p.R98W, p.T96K, p.D87N, p.L211P, p.R136Q, or p.N68K). In some embodiments, the mutation in the TREM2 gene may result from a single nucleotide substitution or deletion (e.g., c.40G>T, c.C97>T, c.132G>A, c.267delGm c.313delG, c.377T>G, c.401A>G, c.482+2T>C, c.558GA).

In some embodiments, the subject suffering from TREM2-associated AD, PLOSL, FTLD, or PD may carry any other pathogenic mutation in the TREM2 gene known to have a causative role in AD, PLOSL, FTLD, or PD. For example, pathogenic mutations in the TREM2 gene may be any of the mutations discussed in Guerreiro et al., The New England Journal of Medicine 368:117-27, (2013); Jonsson et al., The New England Journal of Medicine 368:107-16; Ulrich et al., Neuron Review 94:237-48 (2017); and Xing et al., Research and Reports in Biochemistry 5:89-100 (2015); the disclosures of which are incorporated herein by reference as they pertain to AD-associated and PLOSL-associated human TREM2 mutations.

In some embodiments, the transgene encoding TREM2 contains a polynucleotide encoding of wild type human TREM2 polypeptide (e.g., any one of SEQ ID NOS. 1-3). In some embodiments, the transgene encoding TREM2 includes a polynucleotide encoding a polypeptide having at least 85% sequence identity (e.g., at least 86%, 90%, 95%, 96%, 97%, 98%, 99%, or more, sequence identity) to a polypeptide having the amino acid sequence of any one of SEQ ID NOS. 1-3.

In some embodiments, the transgene encoding TREM2 includes a polynucleotide encoding a polypeptide having at least 85% (e.g. at least 86%, 90%, 95%, 96%, 97%, 98%, 99%, or more) sequence identity to a polypeptide having the amino acid sequence of any one of SEQ ID NO. 1. In some embodiments, the transgene encoding TREM2 includes a polynucleotide encoding a polypeptide having at least 90% (e.g. at least 91%, 95%, 96%, 97%, 98%, 99%, or more) sequence identity to a polypeptide having the amino acid sequence of any one of SEQ ID NO. 1. In some embodiments, the transgene encoding TREM2 includes a polynucleotide encoding a polypeptide having at least 95% (e.g., at least 96%, 97%, 98%, 99%, or more) sequence identity to a polypeptide having the amino acid sequence of any one of SEQ ID NO. 1. In some embodiments, the transgene encoding TREM2 includes a polynucleotide encoding a polypeptide having at least 98% (e.g. at least 99% or more) sequence identity to a polypeptide having the amino acid sequence of any one of SEQ ID NO. 1. In some embodiments, the transgene encoding TREM2 includes a polynucleotide encoding a polypeptide having at least 99% sequence identity to a polypeptide having the amino acid sequence of any one of SEQ ID NO. 1. In some embodiments, the transgene encoding TREM2 includes a polynucleotide encoding a polypeptide having the amino acid sequence of SEQ ID NO. 1.

In some embodiments, the transgene encoding TREM2 includes a polynucleotide encoding a polypeptide having at least 85% (e.g. at least 86%, 90%, 95%, 96%, 97%, 98%, 99%, or more) sequence identity to a polypeptide having the amino acid sequence of any one of SEQ ID NO. 2. In some embodiments, the transgene encoding TREM2 includes a polynucleotide encoding a polypeptide having at least 90% (e.g. at least 91%, 95%, 96%, 97%, 98%, 99%, or more) sequence identity to a polypeptide having the amino acid sequence of any one of SEQ ID NO. 2. In some embodiments, the transgene encoding TREM2 includes a polynucleotide encoding a polypeptide having at least 95% (e.g., at least 96%, 97%, 98%, 99%, or more) sequence identity to a polypeptide having the amino acid sequence of any one of SEQ ID NO. 2. In some embodiments, the transgene encoding TREM2 includes a polynucleotide encoding a polypeptide having at least 98% (e.g. at least 99% or more) sequence identity to a polypeptide having the amino acid sequence of any one of SEQ ID NO. 2. In some embodiments, the transgene encoding TREM2 includes a polynucleotide encoding a polypeptide having at least 99% sequence identity to a polypeptide having the amino acid sequence of any one of SEQ ID NO. 2. In some embodiments, the transgene encoding TREM2 includes a polynucleotide encoding a polypeptide having the amino acid sequence of SEQ ID NO. 2.

In some embodiments, the transgene encoding TREM2 includes a polynucleotide encoding a polypeptide having 85% (e.g. at least 86%, 90%, 95%, 96%, 97%, 98%, 99%, or more) sequence identity to a polypeptide having the amino acid sequence of any one of SEQ ID NO. 3. In some embodiments, the transgene encoding TREM2 includes a polynucleotide encoding a polypeptide having at least 90% (e.g. at least 91%, 95%, 96%, 97%, 98%, 99%, or more) sequence identity to a polypeptide having the amino acid sequence of any one of SEQ ID NO. 3. In some embodiments, the transgene encoding TREM2 includes a polynucleotide encoding a polypeptide having at least 95% (e.g., at least 96%, 97%, 98%, 99%, or more) sequence identity to a polypeptide having the amino acid sequence of any one of SEQ ID NO. 3. In some embodiments, the transgene encoding TREM2 includes a polynucleotide encoding a polypeptide having at least 98% (e.g. at least 99% or more) sequence identity to a polypeptide having the amino acid sequence of any one of SEQ ID NO. 3. In some embodiments, the transgene encoding TREM2 includes a polynucleotide encoding a polypeptide having at least 99% sequence identity to a polypeptide having the amino acid sequence of any one of SEQ ID NO. 3. In some embodiments, the transgene encoding TREM2 includes a polynucleotide encoding a polypeptide having the amino acid sequence of SEQ ID NO. 3.

In some embodiments, the transgene encoding TREM2 includes a polynucleotide encoding polypeptide that contains one or more amino acid substitutions, such as one or more conservative amino acid substitutions (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions), relative to a polypeptide having the sequence of any one of SEQ ID NOS. 1-3.

In some embodiments, the transgene encoding TREM2 includes a polynucleotide encoding a sTREM2 polypeptide. In some embodiments, the transgene encoding TREM2 includes a polynucleotide encoding a TREM2-CTF polypeptide. In some embodiments, the transgene encoding TREM2 includes a polynucleotide encoding a TREM2-ICD polypeptide. In some embodiments, the transgene encoding TREM2 includes a polynucleotide encoding a TREM2-T23 polypeptide. In some embodiments, the transgene encoding TREM2 includes a polynucleotide encoding a TREM2 polypeptide lacking a functional ectodomain cleavage site. In some embodiments, the transgene encoding TREM2 includes a polynucleotide encoding a TREM2 polypeptide lacking a functional intramembrane cleavage site within the TREM2-CTF.

In some embodiments, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments, upon administration of the cells to the subject, expression of the TREM2 transgene is higher in myeloid cells in the subject than in lymphoid cells in the subject. In some embodiments, upon administration of the cells to the subject, expression of the TREM2 transgene is higher in myeloid cells in the subject by at least 50% than in lymphoid cells in the subject. In some embodiments, upon administration of the cells to the subject, expression of the TREM2 transgene is higher in myeloid cells in the subject by at least 60% than in lymphoid cells in the subject. In some embodiments, upon administration of the cells to the subject, expression of the TREM2 transgene is higher in myeloid cells in the subject by at least 70% than in lymphoid cells in the subject. In some embodiments, upon administration of the cells to the subject, expression of the TREM2 transgene is higher in myeloid cells in the subject by at least 80% than in lymphoid cells in the subject. In some embodiments, upon administration of the cells to the subject, expression of the TREM2 transgene is higher in myeloid cells in the subject by at least 90% than in lymphoid cells in the subject. In some embodiments, upon administration of the cells to the subject, expression of the TREM2 transgene is higher in myeloid cells in the subject by at least 95% than in lymphoid cells in the subject. In some embodiments, upon administration of the cells to the subject, expression of the TREM2 transgene is higher in myeloid cells in the subject by at least 100% than in lymphoid cells in the subject. In some embodiments, upon administration of the cells to the subject, expression of the TREM2 transgene is higher in myeloid cells in the subject by at least 150% than in lymphoid cells in the subject. In some embodiments, upon administration of the cells to the subject, expression of the TREM2 transgene is higher in myeloid cells in the subject by at least 200% than in lymphoid cells in the subject. In some embodiments, upon administration of the cells to the subject, expression of the TREM2 transgene is higher in myeloid cells in the subject by at least 250% than in lymphoid cells in the subject. In some embodiments, upon administration of the cells to the subject, expression of the TREM2 transgene is higher in myeloid cells in the subject by at least 300% than in lymphoid cells in the subject. In some embodiments, upon administration of the cells to the subject, expression of the TREM2 transgene is higher in myeloid cells in the subject by at least 350% than in lymphoid cells in the subject. In some embodiments, upon administration of the cells to the subject, expression of the TREM2 transgene is higher in myeloid cells in the subject by at least 400% than in lymphoid cells in the subject. In some embodiments, upon administration of the cells to the subject, expression of the TREM2 transgene is higher in myeloid cells in the subject by at least 450% than in lymphoid cells in the subject. In some embodiments, upon administration of the cells to the subject, expression of the TREM2 transgene is higher in myeloid cells in the subject by at least 500% than in lymphoid cells in the subject.

In some embodiments, the lymphoid cells are B-cells. In some embodiments, the lymphoid cells are T-cells. In some embodiments, the lymphoid cells are natural killer (NK) cells.

In some embodiments, expression of the TREM2 transgene is measured in one or more organs, tissues, or body fluids of the subject. In some embodiments, the one or more body fluids is peripheral blood.

In some embodiments, a population of endogenous microglia in the subject has been ablated prior to administration of the composition to the subject. In some embodiments, the method includes ablating a population of endogenous microglia in the subject prior to administering the composition to the subject. In some embodiments, the microglia are ablated using an agent selected from the group consisting of busulfan, PLX3397, PLX647, PLX5622, treosulfan, and clodronate liposomes, by radiation therapy, or a combination thereof.

In some embodiments, the composition is administered systemically to the subject. In some embodiments, the composition is administered to the subject by way of intravenous injection. In some embodiments, the composition is administered directly to the central nervous system of the subject. In some embodiments, the composition is administered to the cerebrospinal fluid of the subject. For example, the composition may be administered to the subject by way of intracerebroventricular injection, intrathecal injection, stereotactic injection, or a combination thereof. In some embodiments, the composition is administered to the subject by way of intraparenchymal injection.

In some embodiments, the composition is administered to the subject by way of a bone marrow transplant. In some embodiments, the composition is administered directly to the bone marrow of the subject, such as by way of intraosseous injection.

In some embodiments, the composition is administered to the subject by way of intracerebroventricular injection. In some embodiments, the composition is administered to the subject by way of intravenous injection.

In some embodiments, the composition is administered to the subject by direct administration to the central nervous system of the subject and by systemic administration. In some embodiments, the composition is administered to the subject by way of intracerebroventricular injection and intravenous injection. In some embodiments, the composition is administered to the subject by way of intrathecal injection and intravenous injection. In some embodiments, the composition is administered to the subject by way of intraparenchymal injection and intravenous injection.

In some embodiments, the subject is diagnosed with an NCD. In some embodiments, the NCD is a major NCD. In some embodiments, the major NCD interferes with the subject's independence and/or normal daily functioning (e.g., social, occupational, or academic functioning, personal hygiene, grooming, dressing, toilet hygiene, functional mobility (e.g., ability to walk, get in and out of bed), and self-feeding.

In some embodiments, the major NCD is associated with a score obtained by the subject on a cognitive test that is at least two standard deviations away from the mean score of a reference population. In some embodiments, the NCD is a mild NCD. In some embodiments, the mild NCD does not interfere with the subject's independence and/or normal daily functioning. In some embodiments, the mild NCD is associated with a score obtained by the subject on a cognitive test that is between one to two standard deviations away from the mean score of a reference population. In some embodiments, the cognitive test is selected from the group consisting of AD8, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the subject's age, medical history, education, socioeconomic status, and lifestyle. In some embodiments, the NCD is AD. In some embodiments, the NCD is a leukodystrophy. In some embodiments, the leukodystrophy is PLOSL. In some embodiments, the NCD is a frontotemporal NCD. In some embodiments, the frontotemporal NCD is a FTLD. In some embodiments, the FTLD is the behavioral-variant frontotemporal dementia (BVFTD). In some embodiments, the FTLD is the semantic dementia (SD) variant of FTLD. In some embodiments, the FTLD is the progressive nonfluent aphasia (PNA) variant of FTLD. In some embodiments, the NCD is a movement disorder. In some embodiments, the movement disorder is PD.

In some embodiments, the method includes administering to the subject a population of cells. In some embodiments, the population of cells is administered to the subject prior to administration of the composition. In some embodiments, the population of cells is administered to the subject following administration of the composition. In some embodiments, the cells are selected from the group consisting of ESCs, iPSCs, CD34+ cells, HSCs, MPCs, BLPCs, microglial progenitor cells, monocytes, macrophages, and microglia. In some embodiments, the cells are not modified to express a transgene encoding TREM2. In some embodiments, the cells are administered to the subject systemically. In some embodiments, the cells are administered to the subject by way of intravenous injection.

In some embodiments, endogenous TREM2 is disrupted in the cells prior to administration of the composition to the subject.

In some embodiments, the endogenous TREM2 is disrupted by contacting the cells with a nuclease that catalyzes cleavage of an endogenous TREM2 nucleic acid in the cells. In some embodiments, the nuclease is a CRISPR-associated protein. In some embodiments, the CRISPR-associated protein is CRISPR-associated protein 9. In some embodiments, the CRISPR-associated protein is CRISPR-associated protein 12a. In some embodiments, the nuclease is a transcription activator-like effector nuclease, a meganuclease, or a zinc finger nuclease.

In some embodiments, the endogenous TREM2 is disrupted by contacting the cells with an inhibitory RNA molecule, e.g., for a time and in a quantity sufficient to disrupt expression of the endogenous TREM2. In some embodiments, the inhibitory RNA molecule is a short interfering RNA (siRNA), a short hairpin RNA (shRNA), or a miRNA.

In some embodiments, the endogenous TREM2 is disrupted in the subject prior to administration of the composition to the subject. In some embodiments, the endogenous TREM2 is disrupted by administering to the subject an inhibitory RNA molecule. In some embodiments, the inhibitory RNA molecule is a siRNA, a shRNA, or a miRNA. In some embodiments, the endogenous TREM2 is disrupted in a population of neurons in the subject prior to administration of the composition to the subject. In some embodiments, the endogenous TREM2 is disrupted in a population of neurons by contacting the population of neurons with an inhibitory RNA molecule, e.g., for a time and in a quantity sufficient to disrupt expression of the endogenous TREM2. In some embodiments, the inhibitory RNA molecule is a siRNA, a shRNA, or a miRNA.

In some embodiments, the cells are autologous cells. In some embodiments, the cells are allogeneic cells.

In some embodiments, the cells are transduced ex vivo to express the TREM2.

In some embodiments, the cells are transduced with a viral vector selected from the group including an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

In some embodiments, the viral vector is a Retroviridae family viral vector. In some embodiments, the Retroviridae family viral vector is a lentiviral vector. In some embodiments, the Retroviridae family viral vector is an alpharetroviral vector. In some embodiments, the Retroviridae family viral vector is a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is an AAV selected from the group including AAV1, AAV2, AAV3, AAV4, AAV5, AAV6, AAV7, AAV5, AAV9, AAV10, and AAVrh74.

In some embodiments, the viral vector is a pseudotyped viral vector. In some embodiments, the viral vector is a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the viral vector includes a polynucleotide having at least 85% (e.g. at least 86%, 90%, 95%, 96%, 97%, 98%, 99%, or more) sequence identity to the nucleic acid sequence of SEQ ID NO. 15. In some embodiments, the viral vector includes a polynucleotide having at least 90% (e.g. at least 91%, 95%, 96%, 97%, 98%, 99%, or more) sequence identity to the nucleic acid sequence of SEQ ID NO. 15. In some embodiments, the viral vector includes a polynucleotide having at least 95% (e.g., at least 96%, 97%, 98%, 99%, or more) sequence identity to the nucleic acid sequence of SEQ ID NO. 15. In some embodiments, the viral vector includes a polynucleotide having at least 98% (e.g. at least 99% or more) sequence identity to the nucleic acid sequence of SEQ ID NO. 15. In some embodiments, the viral vector includes a polynucleotide having at least 99% sequence identity to the nucleic acid sequence of SEQ ID NO. 15. In some embodiments, the viral vector includes a polynucleotide having the nucleic acid sequence of SEQ ID NO. 15.

In some embodiments, the viral vector includes a polynucleotide having at least 85% (e.g. at least 86%, 90%, 95%, 96%, 97%, 98%, 99%, or more) sequence identity to the nucleic acid sequence of SEQ ID NO. 18. In some embodiments, the viral vector includes a polynucleotide having at least 90% (e.g. at least 91%, 95%, 96%, 97%, 98%, 99%, or more) sequence identity to the nucleic acid sequence of SEQ ID NO. 18. In some embodiments, the viral vector includes a polynucleotide having at least 95% (e.g., at least 96%, 97%, 98%, 99%, or more) sequence identity to the nucleic acid sequence of SEQ ID NO. 18. In some embodiments, the viral vector includes a polynucleotide having at least 98% (e.g. at least 99% or more) sequence identity to the nucleic acid sequence of SEQ ID NO. 18. In some embodiments, the viral vector includes a polynucleotide having at least 99% sequence identity to the nucleic acid sequence of SEQ ID NO. 18. In some embodiments, the viral vector includes a polynucleotide having the nucleic acid sequence of SEQ ID NO. 18.

In some embodiments, the cells are transfected ex vivo to express the TREM2.

In some embodiments, the cells are transfected using an agent selected from the group including a cationic polymer, diethylaminoethyl-dextran, polyethylenimine, a cationic lipid, a liposome, calcium phosphate, an activated dendrimer, and a magnetic bead; or a technique selected from the group including electroporation, Nucleofection, squeeze-poration, sonoporation, optical transfection, Magnetofection, and impalefection.

In some embodiments, the composition is administered to the subject in an amount sufficient to increase the quantity of microglia expressing an anti-inflammatory phenotype in the brain of the subject relative to the quantity of microglia expressing a pro-inflammatory phenotype in the brain of the subject, decrease the level of one or more pro-inflammatory cytokines in the brain of the subject, increase the level of one or more anti-inflammatory cytokines in the brain of the subject, improve the cognitive performance of the subject, improve the motor function of the subject, reduce neuronal loss in the subject, and/or reduce levels of amyloid-3 and neurofibrillary tau proteins, or aggregation thereof, in the subject.

In some embodiments, the subject is a human.

In another aspect, the disclosure provides a composition containing a population of cells that express a transgene encoding TREM2 operably linked to a high expression constitutive promoter. In some embodiments, the high expression constitutive promoter is an MND promoter.

In some embodiments, the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 14. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 14. In some embodiments, the MND promoter includes a polynucleotide having at least 95% (e.g., at least 96%, 97%, 98%, 99%, or more) sequence identity to the nucleic acid sequence of SEQ ID NO. 14. In some embodiments, the MND promoter includes a polynucleotide having at least 98% (e.g., at least 99% or more) sequence identity to the nucleic acid sequence of SEQ ID NO. 14. In some embodiments, the MND promoter includes a polynucleotide having at least 99% sequence identity to the nucleic acid sequence of SEQ ID NO. 14. In some embodiments, the MND promoter includes a polynucleotide having the nucleic acid sequence of SEQ ID NO. 14.

In some embodiments, the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 16. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 16. In some embodiments, the MND promoter includes a polynucleotide having at least 95% (e.g., at least 96%, 97%, 98%, 99%, or more) sequence identity to the nucleic acid sequence of SEQ ID NO. 16. In some embodiments, the MND promoter includes a polynucleotide having at least 98% (e.g., at least 99% or more) sequence identity to the nucleic acid sequence of SEQ ID NO. 16. In some embodiments, the MND promoter includes a polynucleotide having at least 99% sequence identity to the nucleic acid sequence of SEQ ID NO. 16. In some embodiments, the MND promoter includes a polynucleotide having the nucleic acid sequence of SEQ ID NO. 16.

In some embodiments, the high expression constitutive promoter is a CD11b promoter. In some embodiments, the CD11b promoter comprises a polynucleotide having at least 85% (e.g. at least 86%, 90%, 95%, 96%, 97%, 98%, 99%, or more) sequence identity to the nucleic acid sequence of SEQ ID NO. 17. In some embodiments. the CD11b promoter comprises a polynucleotide having at least 90% (e.g. at least 91%, 95%, 96%, 97%, 98%, 99%, or more) sequence identity to the nucleic acid sequence of SEQ ID NO. 17. In some embodiments, the CD11b promoter comprises a polynucleotide having at least 95% (e.g., at least 96%, 97%, 98%, 99%, or more) sequence identity to the nucleic acid sequence of SEQ ID NO. 17. In some embodiments, the CD11b promoter includes a polynucleotide having at least 98% (e.g., at least 99% or more) sequence identity to the nucleic acid sequence of SEQ ID NO. 17. In some embodiments, the CD11b promoter includes a polynucleotide having at least 99% sequence identity to the nucleic acid sequence of SEQ ID NO. 17. In some embodiments, the CD11b promoter includes a polynucleotide having the nucleic acid sequence of SEQ ID NO. 17.

In some embodiments of the preceding aspect, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid cells. In some embodiments, the myeloid cells are myeloid progenitor cells. In some embodiments, the myeloid cells are erythrocytes, mast cells, megakaryocytes, thrombocytes, myeloblasts, basophils, neutrophils, eosinophils, monocytes, or macrophages. In some embodiments, the cells are blood line progenitor cells (BLPCs). In some embodiments, the BLPCs are monocytes. In some embodiments the cells are macrophages. In some embodiments, the cells are microglia.

In some embodiments, upon administration of the composition to a subject, the composition is capable of driving higher expression of the TREM2 transgene in myeloid cells in the subject than in lymphoid cells in the subject. In some embodiments, upon administration of the composition to the subject, expression of the TREM2 transgene is higher in myeloid cells in the subject by at least 50% than in lymphoid cells in the subject. In some embodiments, upon administration of the composition to the subject, expression of the TREM2 transgene is higher in myeloid cells in the subject by at least 60% than in lymphoid cells in the subject. In some embodiments, upon administration of the composition to the subject, expression of the TREM2 transgene is higher in myeloid cells in the subject by at least 70% than in lymphoid cells in the subject. In some embodiments, upon administration of the composition to the subject, expression of the TREM2 transgene is higher in myeloid cells in the subject by at least 80% than in lymphoid cells in the subject. In some embodiments, upon administration of the composition to the subject, expression of the TREM2 transgene is higher in myeloid cells in the subject by at least 90% than in lymphoid cells in the subject. In some embodiments, upon administration of the composition to the subject, expression of the TREM2 transgene is higher in myeloid cells in the subject by at least 95% than in lymphoid cells in the subject. In some embodiments, upon administration of the composition to the subject, expression of the TREM2 transgene is higher in myeloid cells in the subject by at least 100% than in lymphoid cells in the subject. In some embodiments, upon administration of the composition to the subject, expression of the TREM2 transgene is higher in myeloid cells in the subject by at least 150% than in lymphoid cells in the subject. In some embodiments, upon administration of the composition to the subject, expression of the TREM2 transgene is higher in myeloid cells in the subject by at least 200% than in lymphoid cells in the subject. In some embodiments, upon administration of the composition to the subject, expression of the TREM2 transgene is higher in myeloid cells in the subject by at least 250% than in lymphoid cells in the subject. In some embodiments, upon administration of the composition to the subject, expression of the TREM2 transgene is higher in myeloid cells in the subject by at least 300% than in lymphoid cells in the subject. In some embodiments, upon administration of the composition to the subject, expression of the TREM2 transgene is higher in myeloid cells in the subject by at least 350% than in lymphoid cells in the subject. In some embodiments, upon administration of the composition to the subject, expression of the TREM2 transgene is higher in myeloid cells in the subject by at least 400% than in lymphoid cells in the subject. In some embodiments, upon administration of the composition to the subject, expression of the TREM2 transgene is higher in myeloid cells in the subject by at least 450% than in lymphoid cells in the subject. In some embodiments, upon administration of the composition to the subject, expression of the TREM2 transgene is higher in myeloid cells in the subject by at least 500% than in lymphoid cells in the subject.

In some embodiments, the lymphoid cells are B-cells. In some embodiments, the lymphoid cells are T-cells. In some embodiments, the lymphoid cells are NK cells.

In some embodiments, the cells are transduced ex vivo to express the TREM2. In some embodiments, the cells are transfected ex vivo to express the TREM2.

In some embodiments, the TREM2 is full-length TREM2, such as TREM2 having an amino acid sequence of any one of SEQ ID NOS. 1-3 or a variant thereof having at least 85% sequence identity thereto (e.g. at least 86%, 87%, 88%, 89%, 90%, 95%, 96%, 97%, 98%, 99%, or more sequence identity to any one of SEQ ID NOS. 1-3).

In some embodiments, the TREM2 has an amino acid sequence of SEQ ID NO. 1 or is a variant thereof having at least 85% (e.g. at least 86%, 90%, 95%, 96%, 97%, 98%, 99%, or more) sequence identity to the amino acid sequence of SEQ ID NO. 1. In some embodiments, the TREM2 has an amino acid sequence having at least 90% (e.g. at least 91%, 95%, 96%, 97%, 98%, 99%, or more) sequence identity to the amino acid sequence of SEQ ID NO. 1. In some embodiments, the TREM2 has an amino acid sequence having at least 95% (e.g., at least 96%, 97%, 98%, 99%, or more) sequence identity to the amino acid sequence of SEQ ID NO. 1. In some embodiments, the TREM2 has an amino acid sequence having at least 98% (e.g. at least 99% or more) sequence identity to the amino acid sequence of SEQ ID NO. 1. In some embodiments, the TREM2 has an amino acid sequence having at least 99% sequence identity to the amino acid sequence of SEQ ID NO. 1. In some embodiments, the TREM2 has the amino acid sequence of SEQ ID NO. 1.

In some embodiments, the TREM2 has an amino acid sequence of SEQ ID NO. 2 or is a variant thereof having at least 85% (e.g. at least 86%, 90%, 95%, 96%, 97%, 98%, 99%, or more) sequence identity to the amino acid sequence of SEQ ID NO. 2. In some embodiments, the TREM2 has an amino acid having at least 90% (e.g. at least 91%, 95%, 96%, 97%, 98%, 99%, or more) sequence identity to the amino acid sequence of SEQ ID NO. 2. In some embodiments, the TREM2 has an amino acid sequence having at least 95% (e.g., at least 96%, 97%, 98%, 99%, or more) sequence identity to the amino acid sequence of SEQ ID NO. 2. In some embodiments, the TREM2 has an amino acid sequence having at least 98% (e.g. at least 99% or more) sequence identity to the amino acid sequence of SEQ ID NO. 2. In some embodiments, the TREM2 has an amino acid sequence having at least 99% sequence identity to the amino acid sequence of SEQ ID NO. 2. In some embodiments, the TREM2 has the amino acid sequence of SEQ ID NO. 2.

In some embodiments, the TREM2 has an amino acid sequence of SEQ ID NO. 3 or is a variant thereof having at least 85% (e.g. at least 86%, 90%, 95%, 96%, 97%, 98%, 99%, or more) sequence identity to the amino acid sequence of SEQ ID NO. 3. In some embodiments, the TREM2 has an amino acid sequence having at least 90% (e.g. at least 91%, 95%, 96%, 97%, 98%, 99%, or more) sequence identity to the amino acid sequence of SEQ ID NO. 3. In some embodiments, the TREM2 has an amino acid sequence having at least 95% (e.g., at least 96%, 97%, 98%, 99%, or more) sequence identity to the amino acid sequence of SEQ ID NO. 3. In some embodiments, the TREM2 has an amino acid sequence having at least 98% (e.g. at least 99% or more) sequence identity to the amino acid sequence of SEQ ID NO. 3. In some embodiments, the TREM2 has an amino acid sequence having at least 99% sequence identity to the amino acid sequence of SEQ ID NO. 3. In some embodiments, the TREM2 has the amino acid sequence of SEQ ID NO. 3.

In some embodiments, the transgene encoding TREM2 includes a TREM2 polynucleotide having the nucleic acid sequence of SEQ ID NO. 4 or a variant thereof having at least 85% (e.g. at least 86%, 90%, 95%, 96%, 97%, 98%, 99%, or more) sequence identity to the nucleic acid sequence of SEQ ID NO. 4. In some embodiments, the transgene encoding TREM2 includes a TREM2 polynucleotide having the nucleic acid sequence having at least 90% (e.g. at least 91%, 95%, 96%, 97%, 98%, 99%, or more) sequence identity to the nucleic acid sequence of SEQ ID NO. 4. In some embodiments, the transgene encoding TREM2 includes a TREM2 polynucleotide having the nucleic acid sequence having at least 95% (e.g., at least 96%, 97%, 98%, 99%, or more) sequence identity to the nucleic acid sequence of SEQ ID NO. 4. In some embodiments, the transgene encoding TREM2 includes a TREM2 polynucleotide having the nucleic acid sequence having at least 98% (e.g. at least 99% or more) sequence identity to the nucleic acid sequence of SEQ ID NO. 4. In some embodiments, the transgene encoding TREM2 includes a TREM2 polynucleotide having the nucleic acid sequence having at least 99% sequence identity to the nucleic acid sequence of SEQ ID NO. 4. In some embodiments, the transgene encoding TREM2 includes a TREM2 polynucleotide having the nucleic acid sequence of SEQ ID NO. 4.

In some embodiments, the transgene encoding TREM2 includes a TREM2 polynucleotide having the nucleic acid sequence of SEQ ID NO. 5 or a variant thereof having at least 85% (e.g. at least 86%, 90%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 5. In some embodiments, the transgene encoding TREM2 includes a TREM2 polynucleotide having at least 98% (e.g. at least 99% or more) sequence identity to the nucleic acid sequence of SEQ ID NO. 5. In some embodiments, the transgene encoding TREM2 includes a TREM2 polynucleotide having at least 99% sequence identity to the nucleic acid sequence of SEQ ID NO. 5. In some embodiments, the transgene encoding TREM2 includes a TREM2 polynucleotide having the nucleic acid sequence of SEQ ID NO. 5.

In some embodiments, the transgene encoding TREM2 includes a TREM2 polynucleotide having the nucleic acid sequence of SEQ ID NO. 6 or a variant thereof having at least 85% (e.g. at least 86%, 90%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 6. In some embodiments, the transgene encoding TREM2 includes a TREM2 polynucleotide having at least 98% (e.g. at least 99% or more) sequence identity to the nucleic acid sequence of SEQ ID NO. 6. In some embodiments, the transgene encoding TREM2 includes a TREM2 polynucleotide having at least 99% sequence identity to the nucleic acid sequence of SEQ ID NO. 6. In some embodiments, the transgene encoding TREM2 includes a TREM2 polynucleotide having the nucleic acid sequence of SEQ ID NO. 6.

In some embodiments, the transgene encoding TREM2 includes a TREM2 polynucleotide having the nucleic acid sequence of SEQ ID NO. 7 or a variant thereof having at least 85% (e.g. at least 86%, 90%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 7. In some embodiments, the transgene encoding TREM2 includes a TREM2 polynucleotide having at least 98% (e.g. at least 99% or more) sequence identity to the nucleic acid sequence of SEQ ID NO. 7. In some embodiments, the transgene encoding TREM2 includes a TREM2 polynucleotide having at least 99% sequence identity to the nucleic acid sequence of SEQ ID NO. 7. In some embodiments, the transgene encoding TREM2 includes a TREM2 polynucleotide having the nucleic acid sequence of SEQ ID NO. 7.

In some embodiments, the transgene encoding TREM2 includes a TREM2 polynucleotide having the nucleic acid sequence of SEQ ID NO. 9 or a variant thereof having at least 85% (e.g. at least 86%, 90%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 9. In some embodiments, the transgene encoding TREM2 includes a TREM2 polynucleotide having at least 98% (e.g. at least 99% or more) sequence identity to the nucleic acid sequence of SEQ ID NO. 9. In some embodiments, the transgene encoding TREM2 includes a TREM2 polynucleotide having at least 99% sequence identity to the nucleic acid sequence of SEQ ID NO. 9. In some embodiments, the transgene encoding TREM2 includes a TREM2 polynucleotide having the nucleic acid sequence of SEQ ID NO. 9.

In some embodiments, the transgene encoding TREM2 includes a TREM2 polynucleotide having the nucleic acid sequence of SEQ ID NO. 11 or a variant thereof having at least 85% (e.g. at least 86%, 90%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 95% (e.g., at least 96%, 97%, 98%, 99%, or more) sequence identity to the nucleic acid sequence of SEQ ID NO. 11. In some embodiments, the transgene encoding TREM2 includes a TREM2 polynucleotide having at least 98% (e.g. at least 99% or more) sequence identity to the nucleic acid sequence of SEQ ID NO. 11. In some embodiments, the transgene encoding TREM2 includes a TREM2 polynucleotide having at least 99% sequence identity to the nucleic acid sequence of SEQ ID NO. 11. In some embodiments, the transgene encoding TREM2 includes a TREM2 polynucleotide having the nucleic acid sequence of SEQ ID NO. 11.

In some embodiments, the transgene encoding TREM2 may be codon-optimized (e.g., any one of SEQ ID NO. 8, SEQ ID NO. 10, or SEQ ID NO. 12).

In some embodiments, the transgene encoding TREM2 includes a codon-optimized TREM2 polynucleotide of SEQ ID NO. 8 or a variant thereof having at least 85% (e.g. at least 86%, 90%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 98% (e.g. at least 99% or more) sequence identity to the nucleic acid sequence of SEQ ID NO. 8. In some embodiments, the transgene encoding TREM2 includes a codon-optimized TREM2 polynucleotide having at least 99% sequence identity to the nucleic acid sequence of SEQ ID NO. 8. In some embodiments, the transgene encoding TREM2 includes a codon-optimized TREM2 polynucleotide of SEQ ID NO. 8.

In some embodiments, the transgene encoding TREM2 includes a codon-optimized TREM2 polynucleotide of SEQ ID NO. 10 or a variant thereof having at least 85% (e.g. at least 86%, 90%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 98% (e.g. at least 99% or more) sequence identity to the nucleic acid sequence of SEQ ID NO. 10. In some embodiments, the transgene encoding TREM2 includes a codon-optimized TREM2 polynucleotide having at least 99% sequence identity to the nucleic acid sequence of SEQ ID NO. 10. In some embodiments, the transgene encoding TREM2 includes a codon-optimized TREM2 polynucleotide of SEQ ID NO. 10.

In some embodiments, the transgene encoding TREM2 includes a codon-optimized TREM2 polynucleotide of SEQ ID NO. 12 or a variant thereof having at least 85% (e.g. at least 86%, 90%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 95% (e.g., at least 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 98% (e.g. at least 99% or more) sequence identity to the nucleic acid sequence of SEQ ID NO. 12. In some embodiments, the transgene encoding TREM2 includes a codon-optimized TREM2 polynucleotide having at least 99% sequence identity to the nucleic acid sequence of SEQ ID NO. 12. In some embodiments, the transgene encoding TREM2 includes a codon-optimized TREM2 polynucleotide of SEQ ID NO. 12.

In some embodiments, the transgene encodes two or more TREM2 proteins (e.g., at least 2, 3, 4, 5, 6, 7, 8, 9, 10 or more TREM2 proteins). In some embodiments, the transgene encodes from two to ten TREM2 proteins (e.g., 2, 3, 4, 5, 6, 7, 8, 9, or 10 TREM2 proteins). In some embodiments, the transgene encodes from two to five TREM2 proteins (e.g., 2, 3, 4, or 5 TREM2 proteins). In some embodiments, the transgene encodes two TREM2 proteins. In some embodiments, the TREM2 transgenes are expressed from a single, polycistronic expression cassette. In some embodiments, the TREM2 transgenes are separated from one another by way of one or more (e.g., 1, 2, 3, 4, 5, 6, 7, 8, 9, or more) IRES. In some embodiments, the TREM2 transgenes are expressed from one or more (e.g., 1, 2, 3, 4, 5, 6, 7, 8, 9, 10, or more) monocistronic expression cassettes.

In some embodiments, the TREM2 includes a signal peptide. In some embodiments, the signal peptide is a TREM2 signal peptide.

In some embodiments, the TREM2 is sTREM2. In some embodiments, the TREM2 is the TREM-CTF. In some embodiments, the TREM2 is the TREM2-ICD. In some embodiments, the TREM2 is the TREM2-T2β peptide. In some embodiments, the TREM2 lacks a functional ectodomain cleavage site. In some embodiments, the TREM2 lacks a functional intramembrane cleavage site within the TREM2-CTF.

In some embodiments, the TREM2 is a TREM2 fusion protein. In some embodiments, the TREM2 fusion protein includes an Rb domain of ApoE. In some embodiments, the Rb domain includes a portion of ApoE having the amino acid sequence of residues 25-185, 50-180, 75-175, 100-170, 125-160, or 130-150 of SEQ ID NO. 13. In some embodiments, the Rb domain includes a region having at least 70% sequence identity to the amino acid sequence of residues 159-167 of SEQ ID NO. 13.

In some embodiments, the transgene encoding TREM2 further includes a miRNA targeting sequence in the 3′-UTR. In some embodiments, the miRNA targeting sequence is a miR-126 targeting sequence.

In some embodiments, endogenous TREM2 is disrupted in the cells.

In some embodiments, the composition is formulated for systemic administration to the subject. In some embodiments, the composition is formulated for administration to a subject by way of intravenous injection. In some embodiments, the composition is formulated for administration to the cerebrospinal fluid of the subject. In some embodiments, the composition is formulated for administration to a subject by way of intracerebroventricular injection, intrathecal, stereotactic injection, or a combination thereof. In some embodiments, the composition is formulated for administration by way of intraparenchymal injection. In some embodiments, the composition is formulated for administration directly to the bone marrow of a subject. In some embodiments, the composition is formulated for administration to a subject by way of intraosseous injection. In some embodiments, the composition is formulated for administration to a subject by way of bone marrow transplant including the composition. In some embodiments, the composition is formulated for administration to a subject by way of intracerebroventricular injection and intravenous injection.

In another aspect, the disclosure provides a pharmaceutical composition containing compositions according to any of the above aspects and embodiments, the pharmaceutical composition further containing one or more pharmaceutically acceptable carriers, diluents, or excipients.

In an additional aspect, the disclosure provides kits containing compositions according to any of the above aspects and embodiments and a package insert. In some embodiments, the package insert instructs a user of the kit to perform a method according to any of the above aspects and embodiments.

Additional embodiments of the present invention are listed in the enumerated paragraphs below.

E1. A method of treating a subject diagnosed as having a neurocognitive disorder (NCD), the method including administering to the subject a composition including a population of cells (e.g., pluripotent cells, ESCs, iPSCs, multipotent cells, CD34+ cells, HSCs, myeloid cells (e.g., MPCs, macrophages, MPCs, mast cells, erythrocytes, megakaryocytes, thrombocytes, myeloblasts, basophils, neutrophils, eosinophils, or monocytes), BLPCs, microglial progenitor cells, monocytes, macrophages, or microglia) containing a transgene encoding one or more triggering receptor expressed on myeloid cells two (TREM2) proteins operably linked to a high expression constitutive promoter.
E2. The method of E1, wherein the high expression constitutive promoter is a Myeloproliferative Sarcoma Virus Enhancer, Negative Control Region Deleted, dl587rev Primer-Binding Site Substituted (MND) promoter.
E3. The method of E2, wherein the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 14.
E4. The method of E3, wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 14.
E5. The method of E4, wherein the MND promoter includes a polynucleotide having at least 95% (e.g., at least 96%, 97%, 98%, 99%, or more) sequence identity to the nucleic acid sequence of SEQ ID NO. 14.
E6. The method of E5, wherein the MND promoter includes a polynucleotide having the nucleic acid sequence of SEQ ID NO. 14.
E7. The method of E2, wherein the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 16.
E8. The method of E7, wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 16.
E9. The method of E8, wherein the MND promoter includes a polynucleotide having at least 95% (e.g., at least 96%, 97%, 98%, 99%, or more) sequence identity to the nucleic acid sequence of SEQ ID NO. 16.
E10. The method of E9, wherein the MND promoter includes a polynucleotide having the nucleic acid sequence of SEQ ID NO. 16.
E11. The method of E1, wherein the high expression constitutive promoter is a integrin subunit alpha M (CD11b) promoter.
E12. The method of E11, wherein the CD11b promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 17, optionally wherein the CD11b promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 17, optionally wherein the CD11b promoter includes a polynucleotide having at least 95% (e.g., at least 96%, 97%, 98%, 99%, or more) sequence identity to the nucleic acid sequence of SEQ ID NO. 17, optionally wherein the CD11b promoter includes a polynucleotide having the nucleic acid sequence of SEQ ID NO. 17.
E13. The method of any one of E1-E12, wherein the NCD is a major NCD.
E14. The method of E13, wherein the major NCD interferes with the subject's independence and/or normal daily functioning.
E15. The method of E13 or E14, wherein the major NCD is associated with a score obtained by the subject on a cognitive test that is at least two standard deviations away from the mean score of a reference population.
E16. The method of any one of E1-E12, wherein the NCD is a mild NCD.
E17. The method of E16, wherein the mild NCD does not interfere with the subject's independence and/or normal daily functioning.
E18. The method of E16 or E17, wherein the mild NCD is associated with a score obtained by the subject on a cognitive test that is between one to two standard deviations away from the mean score of a reference population.
E19. The method of E15 or E18, wherein the reference population is a general population.
E20. The method of E15, E18, or E19, wherein the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE).
E21. The method of any one of E1-E20, wherein the NCD is associated with impairment in one or more of complex attention, executive function, learning and memory, language, perceptual-motor function, and social cognition.
E22. The method of any one of E1-E21, wherein the NCD is not due to delirium or other mental disorder.
E23. The method of any one of E1-E22, wherein the NCD is Alzheimer's disease (AD).
E24. The method of any one of E1-E22, wherein the NCD is a leukodystrophy.
E25. The method of E24, wherein the leukodystrophy is Nasu-Hakola disease (PLOSL).
E26. The method of any one of E1-E25, wherein the transgene includes a polynucleotide encoding a TREM2 protein having an amino acid sequence that is at least 85% identical to the amino acid sequence of SEQ ID NO. 1.
E27. The method of E26, wherein the transgene includes a polynucleotide encoding a TREM2 protein having an amino acid sequence that is at least 90% identical to the amino acid sequence of SEQ ID NO. 1.
E28. The method of E26, wherein the transgene includes a polynucleotide encoding a TREM2 protein having an amino acid sequence that is at least 95% identical to the amino acid sequence of SEQ ID NO. 1.
E29. The method of E28, wherein the transgene includes a polynucleotide encoding a TREM2 protein having an amino acid sequence of SEQ ID NO. 1.
E30. The method of any one of E1-E29, wherein the transgene includes a polynucleotide encoding a TREM2 protein having an amino acid sequence that is at least 85% identical to the amino acid sequence of SEQ ID NO. 2.
E31. The method of E30, wherein the transgene includes a polynucleotide encoding a TREM2 protein having an amino acid sequence that is at least 90% identical to the amino acid sequence of SEQ ID NO. 2.
E32. The method of E31, wherein the transgene includes a polynucleotide encoding a TREM2 protein having an amino acid sequence that is at least 95% identical to the amino acid sequence of SEQ ID NO. 2.
E33. The method of E32, wherein the transgene includes a polynucleotide encoding a TREM2 protein having an amino acid sequence of SEQ ID NO. 2.
E34. The method of any one of E1-E33, wherein the transgene includes a polynucleotide encoding a TREM2 protein having an amino acid sequence that is at least 85% identical to the amino acid sequence of SEQ ID NO. 3.
E35. The method of E34, wherein the transgene includes a polynucleotide encoding a TREM2 protein having an amino acid sequence that is at least 90% identical to the amino acid sequence of SEQ ID NO. 3.
E36. The method of E35, wherein the transgene includes a polynucleotide encoding a TREM2 protein having an amino acid sequence that is at least 95% identical to the amino acid sequence of SEQ ID NO. 3.
E37. The method of E36, wherein the transgene includes a polynucleotide encoding a TREM2 protein having an amino acid sequence of SEQ ID NO. 3.
E38. The method of any one of E1-E37, wherein the TREM2 is a full-length TREM2.
E39. The method of any one of E1-E38, wherein the TREM2 includes a signal peptide.
E40. The method of E35, wherein the signal peptide is a TREM2 signal peptide.
E41. The method of any one of E1-E40, wherein the TREM2 is a soluble TREM2 (sTREM2).
E42. The method of any one of E1-E40, wherein the TREM2 is a TREM2 C-terminal fragment (TREM2-CTF).
E43. The method of any one of E1-E40, wherein the TREM2 is a TREM2 intracellular domain (TREM2-ICD).
E44. The method of any one of E1-E40, wherein the TREM2 is a TREM2-A p-like (TREM2-T23) peptide.
E45. The method of any one of E1-E40, wherein the TREM2 lacks a functional ectodomain cleavage site.
E46. The method of E42, wherein the TREM2-CTF lacks a functional intramembrane cleavage site.
E47. The method of any one of E1-E46, wherein the transgene includes a polynucleotide encoding two or more TREM2 proteins.
E48. The method of E47, wherein the transgene includes a polynucleotide encoding from two to ten TREM2 proteins.
E49. The method of E48, wherein the transgene includes a polynucleotide encoding from two to five TREM2 proteins.
E50. The method of E49, wherein the transgene includes a polynucleotide encoding two TREM2 proteins.
E51. The method of any one of E47-E50, wherein the TREM2 transgenes are expressed from a single, polycistronic expression cassette.
E52. The method of any one of E47-E51, wherein the TREM2 transgenes are separated from one another by way of one or more internal ribosome entry sites (IRES).
E53. The method of any one of E47-E50, wherein the TREM2 transgenes are expressed from one or more monocistronic expression cassettes.
E54. The method of any one of E1-E53, wherein the transgene includes a polynucleotide having at least 85% (e.g. at least 86%, 90%, 95%, 96%, 97%, 98%, 99%, or more) sequence identity to the nucleic acid sequence of SEQ ID NO. 4.
E55. The method of E54, wherein the transgene includes a polynucleotide having at least 90% (e.g. at least 91%, 95%, 96%, 97%, 98%, 99%, or more) sequence identity to the nucleic acid sequence of SEQ ID NO. 4.
E56. The method of E55, wherein the transgene includes a polynucleotide having at least 95% (e.g., at least 96%, 97%, 98%, 99%, or more) sequence identity to the nucleic acid sequence of SEQ ID NO. 4.
E57. The method of E56, wherein the transgene includes a polynucleotide having the nucleic acid sequence of SEQ ID NO. 4.
E58. The method of any one of E1-E57, wherein the transgene includes a polynucleotide having at least 85% (e.g. at least 86%, 90%, 95%, 96%, 97%, 98%, 99%, or more) sequence identity to the nucleic acid sequence of SEQ ID NO. 5.
E59. The method of E58, wherein the transgene includes a polynucleotide having at least 90% (e.g. at least 91%, 95%, 96%, 97%, 98%, 99%, or more) sequence identity to the nucleic acid sequence of SEQ ID NO. 5.
E60. The method of E59, wherein the transgene includes a polynucleotide having at least 95% (e.g., at least 96%, 97%, 98%, 99%, or more) sequence identity to the nucleic acid sequence of SEQ ID NO. 5.
E61. The method of E60, wherein the transgene includes a polynucleotide having the nucleic acid sequence of SEQ ID NO. 5.
E62. The method of any one of E1-E61, wherein the transgene includes a polynucleotide having at least 85% (e.g. at least 86%, 90%, 95%, 96%, 97%, 98%, 99%, or more) sequence identity to the nucleic acid sequence of SEQ ID NO. 6.
E63. The method of E62, wherein the transgene includes a polynucleotide having at least 90% (e.g. at least 91%, 95%, 96%, 97%, 98%, 99%, or more) sequence identity to the nucleic acid sequence of SEQ ID NO. 6.
E64. The method of E63, wherein the transgene includes a polynucleotide having at least 95% (e.g., at least 96%, 97%, 98%, 99%, or more) sequence identity to the nucleic acid sequence of SEQ ID NO. 6.
E65. The method of E64, wherein the transgene includes a polynucleotide having the nucleic acid sequence of SEQ ID NO. 6.
E66. The method of any one of E1-E65, wherein the transgene includes a polynucleotide having at least 85% (e.g. at least 86%, 90%, 95%, 96%, 97%, 98%, 99%, or more) sequence identity to the nucleic acid sequence of SEQ ID NO. 7.
E67. The method of E66, wherein the transgene includes a polynucleotide having at least 90% (e.g. at least 91%, 95%, 96%, 97%, 98%, 99%, or more) sequence identity to the nucleic acid sequence of SEQ ID NO. 7.
E68. The method of E67, wherein the transgene includes a polynucleotide having at least 95% (e.g., at least 96%, 97%, 98%, 99%, or more) sequence identity to the nucleic acid sequence of SEQ ID NO. 7.
E69. The method of E68, wherein the transgene includes a polynucleotide having the nucleic acid sequence of SEQ ID NO. 7.
E70. The method of any one of E1-E69, wherein the transgene includes a polynucleotide having at least 85% (e.g. at least 86%, 90%, 95%, 96%, 97%, 98%, 99%, or more) sequence identity to the nucleic acid sequence of SEQ ID NO. 9.
E71. The method of E70, wherein the transgene includes a polynucleotide having at least 90% (e.g. at least 91%, 95%, 96%, 97%, 98%, 99%, or more) sequence identity to the nucleic acid sequence of SEQ ID NO. 9.
E72. The method of E71, wherein the transgene includes a polynucleotide having at least 95% (e.g., at least 96%, 97%, 98%, 99%, or more) sequence identity to the nucleic acid sequence of SEQ ID NO. 9.
E73. The method of E72, wherein the transgene includes a polynucleotide having the nucleic acid sequence of SEQ ID NO. 9.
E74. The method of any one of E1-E73, wherein the transgene includes a polynucleotide having at least 85% (e.g. at least 86%, 90%, 95%, 96%, 97%, 98%, 99%, or more) sequence identity to the nucleic acid sequence of SEQ ID NO. 11.
E75. The method of E74, wherein the transgene includes a polynucleotide having at least 90% (e.g. at least 91%, 95%, 96%, 97%, 98%, 99%, or more) sequence identity to the nucleic acid sequence of SEQ ID NO. 11.
E76. The method of E75, wherein the transgene includes a polynucleotide having at least 95% (e.g., at least 96%, 97%, 98%, 99%, or more) sequence identity to the nucleic acid sequence of SEQ ID NO. 11.
E77. The method of E76, wherein the transgene includes a polynucleotide having the nucleic acid sequence of SEQ ID NO. 11.
E78. The method of any one of E1-E77, wherein the transgene is a codon-optimized TREM2 transgene.
E79. The method of E78, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 8.
E80. The method of E79, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 8.
E81. The method of E80, wherein the codon-optimized TREM2 transgene includes a polynucleotide having at least 95% (e.g., at least 96%, 97%, 98%, 99%, or more) sequence identity to the nucleic acid sequence of SEQ ID NO. 8.
E82. The method of E81, wherein the codon-optimized TREM2 transgene includes a polynucleotide of SEQ ID NO. 8.
E83. The method of any one of E78-E82, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 10.
E84. The method of E83, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 10.
E85. The method of E84, wherein the codon-optimized TREM2 transgene includes a polynucleotide having at least 95% (e.g., at least 96%, 97%, 98%, 99%, or more) sequence identity to the nucleic acid sequence of SEQ ID NO. 10.
E86. The method of E85, wherein the codon-optimized TREM2 transgene includes a polynucleotide of SEQ ID NO. 10.
E87. The method of any one of E78-E86, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 12.
E88. The method of E87, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 12.
E89. The method of E88, wherein the codon-optimized TREM2 transgene includes a polynucleotide having at least 95% (e.g., at least 96%, 97%, 98%, 99%, or more) sequence identity to the nucleic acid sequence of SEQ ID NO. 12.
E90. The method of E89, wherein the codon-optimized TREM2 transgene includes a polynucleotide of SEQ ID NO. 12.
E91. The method of any one of E1-E90, wherein the TREM2 is a TREM2 fusion protein.
E92. The method of E91, wherein the TREM2 fusion protein includes a receptor-binding (Rb) domain of apolipoprotein E (ApoE).
E93. The method of E92, wherein the Rb domain includes a portion of ApoE having the amino acid sequence of residues 25-185, 50-180, 75-175, 100-170, 125-160, or 130-150 of SEQ ID NO. 13.
E94. The method of E87 or E88, wherein the Rb domain includes a region having at least 70% (e.g., at least 70%, 75%, 80%, 85%, 90%, 95%, 96%, 97%, 98%, 99%, or more) sequence identity to the amino acid sequence of residues 159-167 of SEQ ID NO. 13.
E95. The method of any one of E1-E94, wherein the transgene encoding TREM2 further includes a microRNA (miRNA) targeting sequence in the 3′-UTR.
E96. The method of E95, wherein the miRNA targeting sequence is a miR-126 targeting sequence.
E97. The method of any one of E1-E96, wherein upon administration of the composition to the subject, the TREM2 penetrates the blood brain barrier in the subject.
E98. The method of any one of E23-E97, wherein the AD or PLOSL is TREM2-associated AD or PLOSL.
E99. The method of any one of E1-E98, wherein the cells are ESCs.
E100. The method of any one of E1-E98, wherein the cells are iPSCs.
E101. The method of any one of E1-E98, wherein the cells are CD34+ cells.
E102. The method of E101, wherein the CD34+ cells are HSCs.
E103. The method of E101, wherein the CD34+ cells are myeloid cells.
E104. The method of E103, wherein the myeloid cells are MPCs.
E105. The method of E103, wherein the myeloid cells are erythrocytes, mast cells, megakaryocytes, thrombocytes, myeloblasts, basophils, neutrophils, eosinophils, monocytes, or macrophages.
E106. The method of any one of E103-E105, wherein, upon administration of the cells to the subject, expression the TREM2 transgene is higher in myeloid cells in the subject than in lymphoid cells in the subject.
E107. The method of E106, wherein, upon administration of the cells to the subject, expression of the TREM2 transgene is higher in myeloid cells in the subject by at least 50% than in lymphoid cells in the subject; optionally, wherein, upon administration of the cells to the subject, expression of the TREM2 transgene is higher in myeloid cells in the subject by at least 60% than in lymphoid cells in the subject; optionally, wherein, upon administration of the cells to the subject, expression of the TREM2 transgene is higher in myeloid cells in the subject by at least 70% than in lymphoid cells in the subject; optionally, wherein, upon administration of the cells to the subject, expression of the TREM2 transgene is higher in myeloid cells in the subject by at least 80% than in lymphoid cells in the subject; optionally, wherein, upon administration of the cells to the subject, expression of the TREM2 transgene is higher in myeloid cells in the subject by at least 90% than in lymphoid cells in the subject; optionally, wherein, upon administration of the cells to the subject, expression of the TREM2 transgene is higher in myeloid cells in the subject by at least 95% than in lymphoid cells in the subject; optionally, wherein, upon administration of the cells to the subject, expression of the TREM2 transgene is higher in myeloid cells in the subject by at least 100% than in lymphoid cells in the subject; optionally, wherein, upon administration of the cells to the subject, expression of the TREM2 transgene is higher in myeloid cells in the subject by at least 150% than in lymphoid cells in the subject; optionally, wherein, upon administration of the cells to the subject, expression of the TREM2 transgene is higher in myeloid cells in the subject by at least 200% than in lymphoid cells in the subject; optionally, wherein, upon administration of the cells to the subject, expression of the TREM2 transgene is higher in myeloid cells in the subject by at least 250% than in lymphoid cells in the subject; optionally, wherein, upon administration of the cells to the subject, expression of the TREM2 transgene is higher in myeloid cells in the subject by at least 300% than in lymphoid cells in the subject; optionally, wherein, upon administration of the cells to the subject, expression of the TREM2 transgene is higher in myeloid cells in the subject by at least 350% than in lymphoid cells in the subject; optionally, wherein, upon administration of the cells to the subject, expression of the TREM2 transgene is higher in myeloid cells in the subject by at least 400% than in lymphoid cells in the subject; optionally, wherein, upon administration of the cells to the subject, expression of the TREM2 transgene is higher in myeloid cells in the subject by at least 450% than in lymphoid cells in the subject; optionally, wherein, upon administration of the cells to the subject, expression of the TREM2 transgene is higher in myeloid cells in the subject by at least 500% than in lymphoid cells in the subject.
E108. The method of E106 or E107, wherein the lymphoid cells are B-cells or T-cells.
E109. The method of any one of E106-E108, wherein the expression level of the TREM2 transgene is measured in one or more organs, tissues, or body fluids of the subject.
E110. The method of E109, wherein the one or more body fluids is peripheral blood.
E111. The method of any one of E1-E110, wherein a population of endogenous microglia in the subject has been ablated prior to administration of the composition.
E112. The method of any one of E1-E110, the method including ablating a population of endogenous microglia in the subject prior to administering the composition to the subject.
E113. The method of E111 or E112 wherein the microglia are ablated using an agent selected from the group consisting of busulfan, PLX3397, PLX647, PLX5622, treosulfan, and clodronate liposomes, by radiation therapy, or a combination thereof.
E114. The method of any one of E1-E113, wherein the composition is administered systemically to the subject.
E115. The method of E114, wherein the composition is administered to the subject by way of intravenous injection.
E116. The method of any one of E1-E113, wherein the composition is administered directly to the central nervous system of the subject.
E117. The method of E116, wherein, the composition is administered to the subject by way of direct administration to the cerebrospinal fluid.
E118. The method of E117, wherein the composition is administered to the subject by way of intracerebroventricular injection, intrathecal injection, stereotactic injection, or a combination thereof.
E119. The method of E116, wherein the composition is administered to the subject by way of intraparenchymal injection.
E120. The method of any one of E1-E113, wherein the composition is administered directly to the bone marrow of the subject.
E121. The method of E120, wherein the composition is administered to the subject by way of intraosseous injection.
E122. The method of any one of E1-E113, wherein the composition is administered to the subject by way of a bone marrow transplant including the composition.
E123. The method of any one of E1-E113, wherein the composition is administered to the subject by way of intracerebroventricular injection.
E124. The method of any one of E1-E113, wherein the composition is administered to the subject by way of intrathecal injection.
E125. The method of any one of E1-E113, wherein the composition is administered to the subject by way of intraparenchymal injection.
E126. The method of any one of E1-E113, wherein the composition is administered to the subject by way of intravenous injection.
E127. The method of any one of E1-E113, wherein the composition is administered to the subject by direct administration to the central nervous system of the subject and by systemic administration.
E128. The method of E127, wherein the composition is administered to the subject by way of intracerebroventricular injection and intravenous injection.
E129. The method of E127, wherein the composition is administered to the subject by way of intrathecal injection and intravenous injection.
E130. The method of E127, wherein the composition is administered to the subject by way of intraparenchymal injection and intravenous injection.
E131. The method of any one of E1-E130, the method further including administering to the subject a population of cells.
E132. The method of E131, wherein the population of cells is administered to the subject prior to administration of the composition.
E133. The method of E131, wherein the population of cells is administered to the subject following administration of the composition.
E134. The method of any one of E131-E133, wherein the cells are selected from the group consisting of pluripotent cells, ESCs, iPSCs, multipotent cells, HSCs, myeloid cells (e.g., MPCs, erythrocytes, mast cells, megakaryocytes, thrombocytes, myeloblasts, basophils, neutrophils, eosinophils, monocytes, or macrophages), BLPCs, monocytes, microglial progenitor cells, macrophages, and microglia.
E135. The method of any one of E131-E134, wherein the cells are not modified to express a transgene encoding TREM2.
E136. The method of any one of E131-E135, wherein the cells are administered to the subject systemically.
E137. The method of E136, wherein the cells are administered to the subject by way of intravenous injection.
E138. The method of any one of E1-E137, wherein, prior to administration of the composition to the subject, endogenous TREM2 is disrupted in the cells.
E139. The method of any one of E1-E138, wherein, prior to administration of the composition to the subject, endogenous TREM2 is disrupted in the subject.
E140. The method of E139, wherein, prior to the administration of the composition to the subject, endogenous TREM2 is disrupted in a population of neurons in the subject.
E141. The method of E138, wherein the endogenous TREM2 is disrupted by contacting the cells with a nuclease that catalyzes cleavage of an endogenous TREM2 nucleic acid in the cells.
E142. The method of E139, wherein the nuclease is a clustered regularly interspaced short palindromic repeats (CRISPR)-associated protein.
E143. The method of E142, wherein the CRISPR-associated protein is CRISPR-associated protein 9 (Cas9).
E144. The method of E142, wherein the CRISPR-associated protein is CRISPR-associated protein 12a (Cas12a)
E145. The method of E141, wherein the nuclease is a transcription activator-like effector nuclease, a meganuclease, or a zinc finger nuclease.
E146. The method of any one of E138-E140, wherein the endogenous TREM2 is disrupted by administering an inhibitory RNA molecule to the cells, the subject, or the population of neurons.
E147. The method of E146, wherein the inhibitory RNA molecule is a short interfering RNA, a short hairpin RNA, or a miRNA.
E148. The method of any one of E1-E147, wherein the cells are autologous cells.
E149. The method of any one of E1-E147, wherein the cells are allogeneic cells.
E150. The method of any one of E1-E149, wherein the cells are transduced ex vivo to express the TREM2.
E151. The method of E150, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.
E152. The method of E151, wherein the viral vector is a Retroviridae family viral vector.
E153. The method of E152, wherein the Retroviridae family viral vector is a lentiviral vector.
E154. The method of E152, wherein the Retroviridae family viral vector is an alpharetroviral vector.
E155. The method of E152, wherein the Retroviridae family viral vector is a gammaretroviral vector.
E156. The method of any one of E152-E155, wherein the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E157. The method of E151, wherein the viral vector is an AAV selected from the group consisting of AAV1, AAV2, AAV3, AAV4, AAV5, AAV6, AAV7, AAV8, AAV9, AAV10, and AAVrh74.
E158. The method of any one of E151-E157, wherein the viral vector is a pseudotyped viral vector.
E159. The method of E158, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E160. The method of any one of E151-E159, wherein the viral vector includes a polynucleotide having at least 85% (e.g. at least 86%, 90%, 95%, 96%, 97%, 98%, 99%, or more) sequence identity to the nucleic acid sequence of SEQ ID NO. 15, optionally wherein the viral vector includes a polynucleotide having at least 90% (e.g. at least 91%, 95%, 96%, 97%, 98%, 99%, or more) sequence identity to the nucleic acid sequence of SEQ ID NO. 15, optionally wherein the viral vector includes a polynucleotide includes a polynucleotide having at least 95% (e.g., at least 96%, 97%, 98%, 99%, or more) sequence identity to the nucleic acid sequence of SEQ ID NO. 15, optionally wherein the viral vector includes a polynucleotide includes a polynucleotide having at least 98% (e.g. at least 99% or more) sequence identity to the nucleic acid sequence of SEQ ID NO. 15, optionally wherein the viral vector includes a polynucleotide includes a polynucleotide having at least 99% sequence identity to the nucleic acid sequence of SEQ ID NO. 15, optionally wherein the viral vector includes a polynucleotide includes a polynucleotide having the nucleic acid sequence of SEQ ID NO. 15.
E161. The method of any one of E151-E159, wherein the viral vector includes a polynucleotide having at least 85% (e.g. at least 86%, 90%, 95%, 96%, 97%, 98%, 99%, or more) sequence identity to the nucleic acid sequence of SEQ ID NO. 18, optionally wherein the viral vector includes a polynucleotide having at least 90% (e.g. at least 91%, 95%, 96%, 97%, 98%, 99%, or more) sequence identity to the nucleic acid sequence of SEQ ID NO. 18, optionally wherein the viral vector includes a polynucleotide includes a polynucleotide having at least 95% (e.g., at least 96%, 97%, 98%, 99%, or more) sequence identity to the nucleic acid sequence of SEQ ID NO. 18, optionally wherein the viral vector includes a polynucleotide includes a polynucleotide having at least 98% (e.g. at least 99% or more) sequence identity to the nucleic acid sequence of SEQ ID NO. 18, optionally wherein the viral vector includes a polynucleotide includes a polynucleotide having at least 99% sequence identity to the nucleic acid sequence of SEQ ID NO. 18, optionally wherein the viral vector includes a polynucleotide includes a polynucleotide having the nucleic acid sequence of SEQ ID NO. 18.
E162. The method of any one of E1-E161, wherein the cells are transfected ex vivo to express the TREM2.
E163. The method of E162, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.
E164. The method of any one of E1-E163, wherein the composition is administered to the subject in an amount sufficient to: a) increase the quantity of microglia expressing an anti-inflammatory phenotype in the brain of the subject relative to the quantity of microglia expressing a pro-inflammatory phenotype in the brain of the subject; b) decrease the level of one or more pro-inflammatory cytokines in the brain of the subject; c) increase the level of one or more anti-inflammatory cytokines in the brain of the subject; d) improve the cognitive performance of the subject; e) improve the motor function of the subject; f) reduce neuron loss in the subject; and/or g) reduce levels of amyloid-3 and neurofibrillary tau proteins, or aggregation thereof in the subject.
E165. The method of any one of E1-E164, wherein the subject is a human.
E166. A composition including a population of cells containing a transgene encoding TREM2 (e.g., a transgene capable of expression in macrophages or microglial cells) operably linked to a high expression constitutive promoter.
E167. The composition of E166, wherein the high expression constitutive promoter is an MND promoter.
E168. The composition of E167, wherein the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 14.
E169. The composition of E168, wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 14.
E170. The composition of E169, wherein the MND promoter includes a polynucleotide having at least 95% (e.g., at least 96%, 97%, 98%, 99%, or more) sequence identity to the nucleic acid sequence of SEQ ID NO. 14.
E171. The composition of E170, wherein the MND promoter includes a polynucleotide having the nucleic acid sequence of SEQ ID NO. 14.
E172. The composition of E167, wherein the MND promoter includes a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 16.
E173. The composition of E172, wherein the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO. 16.
E174. The composition of E173, wherein the MND promoter includes a polynucleotide having at least 95% (e.g., at least 96%, 97%, 98%, 99%, or more) sequence identity to the nucleic acid sequence of SEQ ID NO. 16.
E175. The composition of E174, wherein the MND promoter includes a polynucleotide having the nucleic acid sequence of SEQ ID NO. 16.
E176. The composition of E166, wherein the high expression constitutive promoter is a integrin subunit alpha M (CD11b) promoter.
E177. The composition of E176, wherein the CD11b promoter includes a polynucleotide having at least 85% (e.g. at least 86%, 90%, 95%, 96%, 97%, 98%, 99%, or more) sequence identity to the nucleic acid sequence of SEQ ID NO. 17, optionally wherein the CD11b promoter includes a polynucleotide having at least 90% (e.g. at least 91%, 95%, 96%, 97%, 98%, 99%, or more) sequence identity to the nucleic acid sequence of SEQ ID NO. 17, optionally wherein the CD11b promoter includes a polynucleotide having at least 95% (e.g., at least 96%, 97%, 98%, 99%, or more) sequence identity to the nucleic acid sequence of SEQ ID NO. 17, optionally wherein the CD11b promoter includes a polynucleotide having the nucleic acid sequence of SEQ ID NO. 17.
E178. The composition of any one of E166-E177, wherein the TREM2 is a full-length TREM2.
E179. The composition of any one of E166-E178, wherein the TREM2 or a variant thereof has an amino acid sequence with at least 85% (e.g., at least 86%, 90%, 95%, 96%, 97%, 98%, 99%, or more) sequence identity to the amino acid sequence of any one of SEQ ID NOS. 1-3.
E180. The composition of E179, wherein the TREM2 has an amino acid sequence that has at least 85% (e.g. at least 86%, 90%, 95%, 96%, 97%, 98%, 99%, or more) sequence identity to the amino acid sequence of SEQ ID NO. 1.
E181. The composition of E180, wherein the TREM2 has an amino acid sequence that has at least 90% (e.g. at least 91%, 95%, 96%, 97%, 98%, 99%, or more) sequence identity to the amino acid sequence of SEQ ID NO. 1.
E182. The composition of E181, wherein the TREM2 has an amino acid sequence that has at least 95% (e.g., at least 96%, 97%, 98%, 99%, or more) sequence identity to the amino acid sequence of SEQ ID NO. 1.
E183. The composition of E182, wherein the TREM2 has the amino acid sequence of SEQ ID NO. 1.
E184. The composition of any one of E166-E183, wherein the TREM2 has an amino acid sequence that has at least 85% (e.g. at least 86%, 90%, 95%, 96%, 97%, 98%, 99%, or more) sequence identity to the amino acid sequence of SEQ ID NO. 2.
E185. The composition of E184, wherein the TREM2 has an amino acid sequence that has at least 90% (e.g. at least 91%, 95%, 96%, 97%, 98%, 99%, or more) sequence identity to the amino acid sequence of SEQ ID NO. 2.
E186. The composition of E185, wherein the TREM2 has an amino acid sequence that has at least 95% (e.g., at least 96%, 97%, 98%, 99%, or more) sequence identity to the amino acid sequence of SEQ ID NO. 2.
E187. The composition of E186, wherein the TREM2 has the amino acid sequence of SEQ ID NO. 2.
E188. The composition of any one of E166-E187, wherein the TREM2 has an amino acid sequence that has at least 85% (e.g. at least 86%, 90%, 95%, 96%, 97%, 98%, 99%, or more) sequence identity of SEQ ID NO. 3.
E189. The composition of E188, wherein the TREM2 has an amino acid sequence that has at least 90% (e.g. at least 91%, 95%, 96%, 97%, 98%, 99%, or more) sequence identity to the amino acid sequence of SEQ ID NO. 3.
E190. The composition of E189, wherein the TREM2 has an amino acid sequence that has at least 95% (e.g., at least 96%, 97%, 98%, 99%, or more) sequence identity to the amino acid sequence of SEQ ID NO. 3.
E191. The composition of E190, wherein the TREM2 has the amino acid sequence of SEQ ID NO. 3.
E192. The composition of any one of E166-E191, wherein the TREM2 includes a signal peptide.
E193. The composition of E192, wherein the signal peptide is a TREM2 signal peptide.
E194. The composition of any one of E166-E192, wherein the TREM2 is a sTREM2.
E195. The composition of any one of E166-E194, wherein the TREM2 is a TREM2-CTF.
E196. The composition of any one of E166-E195, wherein the TREM2 is a TREM2-ICD.
E197. The composition of any one of E166-E196, wherein the TREM2 is a TREM2-T2β peptide.
E198. The composition of any one of E166-E197, wherein the TREM2 lacks a functional ectodomain cleavage site.
E199. The composition of any one of E166-E198, wherein the TREM2-CTF lacks a functional intramembrane cleavage site.
E200. The composition of any one of E166-E199, wherein the transgene encodes two or more TREM2 proteins.
E201. The composition of E187, wherein the transgene encodes from two to ten TREM2 proteins.
E202. The composition of E188, wherein the transgene encodes from two to five TREM2 proteins.
E203. The composition of E189, wherein the transgene encodes two TREM2 proteins.
E204. The composition of any one of E200-E203, wherein the TREM2 transgenes are expressed from a single, polycistronic expression cassette.
E205. The composition of any one of E200-E203, wherein the TREM2 transgenes are separated from one another by way of one or more IRES.
E206. The composition of any one of E200-E203, wherein the TREM2 transgenes are expressed from one or more monocistronic expression cassettes.
E207. The composition of any one of E166-E206, wherein the transgene includes a polynucleotide having at least 85% (e.g. at least 86%, 90%, 95%, 96%, 97%, 98%, 99%, or more) sequence identity to the nucleic acid sequence of SEQ ID NO. 4.
E208. The composition of E207, wherein the transgene includes a polynucleotide having at least 90% (e.g. at least 91%, 95%, 96%, 97%, 98%, 99%, or more) sequence identity to the nucleic acid sequence of SEQ ID NO. 4.
E209. The composition of E208, wherein the transgene includes a polynucleotide having at least 95% (e.g., at least 96%, 97%, 98%, 99%, or more) sequence identity to the nucleic acid sequence of SEQ ID NO. 4.
E210. The composition of E209, wherein the transgene includes a polynucleotide having the nucleic acid sequence of SEQ ID NO. 4.
E211. The composition of any one of E166-E210, wherein the transgene includes a polynucleotide having at least 85% (e.g. at least 86%, 90%, 95%, 96%, 97%, 98%, 99%, or more) sequence identity to the nucleic acid sequence of SEQ ID NO. 5.
E212. The composition of E211, wherein the transgene includes a polynucleotide having at least 90% (e.g. at least 91%, 95%, 96%, 97%, 98%, 99%, or more) sequence identity to the nucleic acid sequence of SEQ ID NO. 5.
E213. The composition of E212, wherein the transgene includes a polynucleotide having at least 95% (e.g., at least 96%, 97%, 98%, 99%, or more) sequence identity to the nucleic acid sequence of SEQ ID NO. 5.
E214. The composition of E213, wherein the transgene includes a polynucleotide having the nucleic acid sequence of SEQ ID NO. 5.
E215. The composition of any one of E166-E214, wherein the transgene includes a polynucleotide having at least 85% (e.g. at least 86%, 90%, 95%, 96%, 97%, 98%, 99%, or more) sequence identity to the nucleic acid sequence of SEQ ID NO. 6.
E216. The composition of E215, wherein the transgene includes a polynucleotide having at least 90% (e.g. at least 91%, 95%, 96%, 97%, 98%, 99%, or more) sequence identity to the nucleic acid sequence of SEQ ID NO. 6.
E217. The composition of E216, wherein the transgene includes a polynucleotide having at least 95% (e.g., at least 96%, 97%, 98%, 99%, or more) sequence identity to the nucleic acid sequence of SEQ ID NO. 6.
E218. The composition of E217, wherein the transgene includes a polynucleotide having the nucleic acid sequence of SEQ ID NO. 6.
E219. The composition of any one of E166-E218, wherein the transgene includes a polynucleotide having at least 85% (e.g. at least 86%, 90%, 95%, 96%, 97%, 98%, 99%, or more) sequence identity to the nucleic acid sequence of SEQ ID NO. 7.
E220. The composition of E219, wherein the transgene includes a polynucleotide having at least 90% (e.g. at least 91%, 95%, 96%, 97%, 98%, 99%, or more) sequence identity to the nucleic acid sequence of SEQ ID NO. 7.
E221. The composition of E220, wherein the transgene includes a polynucleotide having at least 95% (e.g., at least 96%, 97%, 98%, 99%, or more) sequence identity to the nucleic acid sequence of SEQ ID NO. 7.
E222. The composition of E221, wherein the transgene includes a polynucleotide having the nucleic acid sequence of SEQ ID NO. 7.
E223. The composition of any one of E166-E222, wherein the transgene includes a polynucleotide having at least 85% (e.g. at least 86%, 90%, 95%, 96%, 97%, 98%, 99%, or more) sequence identity to the nucleic acid sequence of SEQ ID NO. 9.
E224. The composition of E223, wherein the transgene includes a polynucleotide having at least 90% (e.g. at least 91%, 95%, 96%, 97%, 98%, 99%, or more) sequence identity to the nucleic acid sequence of SEQ ID NO. 9.
E225. The composition of E224, wherein the transgene includes a polynucleotide having at least 95% (e.g., at least 96%, 97%, 98%, 99%, or more) sequence identity to the nucleic acid sequence of SEQ ID NO. 9.
E226. The composition of E225, wherein the transgene includes a polynucleotide having the nucleic acid sequence of SEQ ID NO. 11.
E227. The composition of any one of E166-E226, wherein the transgene includes a polynucleotide having at least 85% (e.g. at least 86%, 90%, 95%, 96%, 97%, 98%, 99%, or more) sequence identity to the nucleic acid sequence of SEQ ID NO. 11.
E228. The composition of E227, wherein the transgene includes a polynucleotide having at least 90% (e.g. at least 91%, 95%, 96%, 97%, 98%, 99%, or more) sequence identity to the nucleic acid sequence of SEQ ID NO. 11.
E229. The composition of E228, wherein the transgene includes a polynucleotide having at least 95% (e.g., at least 96%, 97%, 98%, 99%, or more) sequence identity to the nucleic acid sequence of SEQ ID NO. 11.
E230. The composition of E229, wherein the transgene includes a polynucleotide having the nucleic acid sequence of SEQ ID NO. 11.
E231. The composition of any one of E166-E230, wherein the transgene is a codon-optimized TREM2 transgene.
E232. The composition of E231, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 8.
E233. The composition of E232, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 8.
E234. The composition of E233, wherein the codon-optimized TREM2 transgene includes a polynucleotide having at least 95% (e.g., at least 96%, 97%, 98%, 99%, or more) sequence identity to the nucleic acid sequence of SEQ ID NO. 8.
E235. The composition of E234, wherein the codon-optimized TREM2 transgene includes a polynucleotide of SEQ ID NO. 8.
E236. The composition of any one of E231-E235, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 10.
E237. The composition of E236, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 10.
E238. The composition of E237, wherein the codon-optimized TREM2 transgene includes a polynucleotide having at least 95% (e.g., at least 96%, 97%, 98%, 99%, or more) sequence identity to the nucleic acid sequence of SEQ ID NO. 10.
E239. The composition of E238, wherein the codon-optimized TREM2 transgene includes a polynucleotide of SEQ ID NO. 10.
E240. The composition of any one of E231-E239, wherein the codon-optimized TREM2 transgene includes a polynucleotide having at least 85% (e.g. at least 86%, 90%, 95%, 96%, 97%, 98%, 99%, or more) sequence identity to the nucleic acid sequence of SEQ ID NO. 12.
E241. The composition of E240, wherein the codon-optimized TREM2 transgene includes a polynucleotide having at least 90% (e.g. at least 91%, 95%, 96%, 97%, 98%, 99%, or more) sequence identity to the nucleic acid sequence of SEQ ID NO. 12.
E242. The composition of E241, wherein the codon-optimized TREM2 transgene includes a polynucleotide having at least 95% (e.g., at least 96%, 97%, 98%, 99%, or more) sequence identity to the nucleic acid sequence of SEQ ID NO. 12.
E243. The composition of E242, wherein the codon-optimized TREM2 transgene includes a polynucleotide of SEQ ID NO. 12.
E244. The composition of any one of E166-E243, wherein the TREM2 is a TREM2 fusion protein.
E245. The composition of E244, wherein the TREM2 fusion protein includes a Rb domain of ApoE.
E246. The composition of E245, wherein the Rb domain includes a portion of ApoE having the amino acid sequence of residues 25-185, 50-180, 75-175, 100-170, 125-160, or 130-150 of SEQ ID NO. 13.
E247. The composition of E245 or E246, wherein the Rb domain includes a region having at least 70% sequence identity to the amino acid sequence of residues 159-167 of SEQ ID NO. 13.
E248. The composition of any one of E166-E247, wherein the transgene encoding TREM2 further includes a miRNA targeting sequence in the 3′-UTR.
E249. The composition of E248, wherein the miRNA targeting sequence is a miR-126 targeting sequence.
E250. The composition of any one of E166-E249, wherein the cells are ESCs (such as, e.g., ESCs that have been differentiated into macrophages or microglia).
E251. The composition of any one of E166-E249, wherein the cells are iPSCs (such as, e.g., iPSCs that have been differentiated into macrophages or microglia).
E252. The composition of any one of E166-E249, wherein the cells are CD34+ cells.
E253. The composition of E252, wherein the CD34+ cells are HSCs.
E254. The composition of E252, wherein the CD34+ cells are myeloid cells.
E255. The composition of E254, wherein the myeloid cells are MPCs.
E256. The composition of E254, wherein the myeloid cells are erythrocytes, mast cells, megakaryocytes, thrombocytes, myeloblasts, basophils, neutrophils, eosinophils, monocytes, or macrophages.
E257. The composition of any one of E166-E256, wherein, upon administration of the composition to a subject, expression of the TREM2 transgene is higher in myeloid cells in the subject than in lymphoid cells in the subject.
E258. The composition of E257, wherein, upon administration of the composition to the subject, expression of the TREM2 transgene is higher in myeloid cells in the subject by at least 50% than in lymphoid cells in the subject; optionally, wherein, upon administration of the composition to the subject, expression of the TREM2 transgene is higher in myeloid cells in the subject by at least 60% than in lymphoid cells in the subject; optionally, wherein, upon administration of the composition to the subject, expression of the TREM2 transgene is higher in myeloid cells in the subject by at least 70% than in lymphoid cells in the subject; optionally, wherein, upon administration of the composition to the subject, expression of the TREM2 transgene is higher in myeloid cells in the subject by at least 80% than in lymphoid cells in the subject; optionally, wherein, upon administration of the composition to the subject, expression of the TREM2 transgene is higher in myeloid cells in the subject by at least 90% than in lymphoid cells in the subject; optionally, wherein, upon administration of the composition to the subject, expression of the TREM2 transgene is higher in myeloid cells in the subject by at least 95% than in lymphoid cells in the subject; optionally, wherein, upon administration of the composition to the subject, expression of the TREM2 transgene is higher in myeloid cells in the subject by at least 100% than in lymphoid cells in the subject; optionally, wherein, upon administration of the composition to the subject, expression of the TREM2 transgene is higher in myeloid cells in the subject by at least 150% than in lymphoid cells in the subject; optionally, wherein, upon administration of the composition to the subject, expression of the TREM2 transgene is higher in myeloid cells in the subject by at least 200% than in lymphoid cells in the subject; optionally, wherein, upon administration of the composition to the subject, expression of the TREM2 transgene is higher in myeloid cells in the subject by at least 250% than in lymphoid cells in the subject; optionally, wherein, upon administration of the composition to the subject, expression of the TREM2 transgene is higher in myeloid cells in the subject by at least 300% than in lymphoid cells in the subject; optionally, wherein, upon administration of the composition to the subject, expression of the TREM2 transgene is higher in myeloid cells in the subject by at least 350% than in lymphoid cells in the subject; optionally, wherein, upon administration of the composition to the subject, expression of the TREM2 transgene is higher in myeloid cells in the subject by at least 400% than in lymphoid cells in the subject; optionally, wherein, upon administration of the composition to the subject, expression of the TREM2 transgene is higher in myeloid cells in the subject by at least 450% than in lymphoid cells in the subject; optionally, wherein, upon administration of the composition to the subject, expression of the TREM2 transgene is higher in myeloid cells in the subject by at least 500% than in lymphoid cells in the subject.
E259. The composition of E257 or E258, wherein the lymphoid cells are B-cells.
E260. The composition of E257 or E258, wherein the lymphoid cells are T-cells.
E261. The composition of any one of E166-E256, wherein the cells are transfected ex vivo to express the TREM2.
E262. The composition of any one of E166-E261, wherein the cells are transduced ex vivo to express TREM2.
E263. The composition of any one of E166-E262, wherein the composition is formulated for systemic administration to a human subject.
E264. The composition of any one of E263, wherein the composition is formulated for administration to a human subject by way of intravenous injection.
E265. The composition of any one of E166-E262, wherein the composition is formulated for administration to a human subject directly to the nervous system of the subject.
E266. The composition of E265, wherein the composition is formulated for administration to a human subject to the cerebrospinal fluid.
E267. The composition of E265 or E266, wherein the composition is formulated for administration to a human subject by way of intracerebroventricular injection, intrathecal, stereotactic injection, or a combination thereof.
E268. The composition of E265, wherein the composition is formulated for administration by way of intraparenchymal injection.
E269. The composition of any one of E166-E262, wherein the composition is formulated for administration directly to the bone marrow of a human subject.
E270. The composition of E269, wherein the composition is formulated for administration to a human subject by way of intraosseous injection.
E271. The composition of any one of E270, wherein the composition is formulated for administration to a human subject by way of a bone marrow transplant including the composition.
E272. The composition of any one of E166-E262, wherein the composition is formulated for administration to the subject by direct administration to the central nervous system of the subject and by systemic administration.
E273. The composition of E272, wherein the composition is formulated for administration by way of intracerebroventricular injection and intravenous injection.
E274. The composition of E272, wherein the composition is formulated for administration by way of intrathecal injection and intravenous injection.
E275. The composition of E272, wherein the composition is formulated for administration by way of intraparenchymal injection and intravenous injection.
E276. The composition of any one of E259-E275, wherein the subject is diagnosed with an NCD.
E277. The composition of E276, wherein the NCD is a major NCD.
E278. The composition of E277, wherein the major NCD interferes with the subject's independence and/or normal daily functioning.
E279. The composition of E276 or E277, wherein the major NCD is associated with a score obtained by the subject on a cognitive test that is at least two standard deviations away from the mean score of a reference population.
E280. The composition of E276, wherein the NCD is a mild NCD.
E281. The composition of E280, wherein the mild NCD does not interfere with the subject's independence and/or normal daily functioning.
E282. The composition of E280 or E281, wherein the mild NCD is associated with a score obtained by the subject on a cognitive test that is between one to two standard deviations away from the mean score of a reference population.
E283. The composition of E279 or E282, wherein the reference population is a general population.
E284. The composition of E279, E282, or E283, wherein the cognitive test is selected from the group consisting of AD8, AWV, GPCOG, HRA, MIS, MMSE, MoCA, SLUMS, and Short IQCODE.
E285. The composition of any one of E276-E284, wherein the NCD is associated with impairment in one or more of complex attention, executive function, learning and memory, language, perceptual-motor function, and social cognition.
E286. The composition of any one of E276-E285, wherein the NCD is not due to delirium or other mental disorder.
E287. The composition of any one of E276-E286, wherein the NCD is Alzheimer's disease (AD).
E288. The composition of any one of E276-E286, wherein the NCD is a leukodystrophy.
E289. The composition of E288, wherein the leukodystrophy is Nasu-Hakola disease (PLOSL).
E290. A pharmaceutical composition including the composition of any one of E166-289, wherein the pharmaceutical composition further includes a pharmaceutically acceptable carrier, diluent, or excipient.
E291. A kit including the composition of any one of E166-E289, or the pharmaceutical composition of E290, and a package insert.
E292. The kit of E291, wherein the package insert instructs a user of the kit to perform the method of any one of E1-E165.
E293. The method of any one of E1-E165, wherein the NCD is a frontotemporal NCD.
E294. The method of E293, wherein the frontotemporal NCD is a FTLD (e.g., BVFTD, SD, or PNA).
E295. The method of any one of E1-E165, wherein the NCD is a movement disorder.
E296. The method of E295, wherein the movement disorder is PD.
E297. The method of any one of E1-E165, wherein the cells are pluripotent cells (e.g., ESCs, iPSCs), multipotent cells (e.g., CD34+ cells, such as, e.g., HSCs or myeloid cells (e.g., MPCs, erythrocytes, mast cells, megakaryocytes, thrombocytes, myeloblasts, basophils, neutrophils, eosinophils, monocytes, or macrophages), BLPCs, monocytes, macrophages, microglial progenitor cells, or microglia.
E298. The method of any one of E1-E165, wherein the transgene is capable of expression in a macrophage or a microglial cell.
E299. The composition of any one of E166-E290, wherein the NCD is a frontotemporal NCD.
E300. The composition of E299, wherein the frontotemporal NCD is FTLD (e.g., BVFTD, SD, or PNA).
E301. The composition of any one of E166-E290, wherein the NCD is a movement disorder.
E302. The composition of any one of E166-E290, wherein the movement disorder is PD.
E303. The composition of any one of E166-E290, wherein the cells are pluripotent cells (e.g., ESCs, iPSCs), multipotent cells (e.g., CD34+ cells, such as, e.g., HSCs or myeloid cells (e.g., MPCs, erythrocytes, mast cells, megakaryocytes, thrombocytes, myeloblasts, basophils, neutrophils, eosinophils, monocytes, or macrophages), BLPCs, monocytes, macrophages, microglial progenitor cells, or microglia.
E304. The composition of any one of E166-E290, wherein the transgene is capable of expression in a macrophage or a microglial cell.

### BRIEF DESCRIPTION OF THE DRAWINGS

FIG. 1 is a Western blot showing expression of the human triggering receptor expressed on myeloid cells 2 (TREM2) protein in a murine macrophage cell line transduced with a lentiviral vector encoding TREM2. Cell lysates were generated from the RAW murine macrophage cells transduced with an MND.TREM2 viral vector (MND.TREM2), an MND.green fluorescent protein (GFP) viral vector (MND.GFP) at multiplicity of infection (MOI) of 10, 50, 100, or 200, or from non-transduced control (NTC) cells. TREM2 expression was assessed using an antibody raised against human TREM2 (FIG. 1).

FIG. 2 is a Western blot showing expression of the human TREM2 protein in murine microglial cells transduced with a lentiviral vector encoding TREM2. Cell lysates were generated from primary murine microglia non-transduced (NT) or transduced with an MND.TREM2 viral vector (MND-TREM2) or an MND.GFP viral vector (MND-GFP). TREM2 expression was assessed using an antibody raised against human TREM2 (FIG. 2).

FIG. 3 is a Western blot showing expression of the human TREM2 protein in lineage negative (Lin−) cells transduced with a lentiviral vector encoding TREM2. Cell lysates from Lin− murine cells transduced with an MND.TREM2 viral vector (Lenti TREM2) or an MND.GFP viral vector. TREM2 expression was assessed using an antibody raised against human TREM2 (FIG. 3).

FIGS. 4A-4D are a series of bar plots showing human TREM2 (hTREM2) expression in peripheral blood of mice treated with a lentiviral plasmid vector (SEQ ID NO. 15) encoding a codon-optimized TREM2 transgene (SEQ ID NO. 8) operably linked to an MND promoter (“MND.TREM2”; SEQ ID NO. 14). For comparison, the bar plots also show the percentage of TREM2-positive cells measured in peripheral blood of mice 30 days after treatment with a plasmid vector encoding green fluorescent protein (GFP) operably linked to the MND promoter (“MND.GFP”). Heterologous TREM2 transgene expression was observed almost exclusively in cells of the myeloid lineage (FIG. 4A). GFP expression was also assessed in the peripheral blood of mice treated with the MND.GFP plasmid vector. The percentage of GFP-positive cells showed no statistically significant differences between myeloid cells, B-cells, and T-cells (FIG. 4B). Therefore, unlike the MND.GFP vector, the MND.TREM2 exhibited myeloid cell-specific transgene expression. Bar graph showing vector copy number (VCN) in peripheral blood following treatment with the TREM2-encoding vector (FIG. 4D). Bar graph showing hTREM2 protein expression in peripheral blood 60 days following treatment with the TREM2-encoding vector (FIG. 4C).

FIGS. 5A-5C show a series of heat maps showing cell type-specific differences in expression of GFP under regulatory control of different promoter sequences (e.g., a phosphoglycerate kinase (PGK) promoter, embryonal fyn-associated substrate (EFS) promoter, MND (SEQ ID NO. 14) promoter, and integrin subunit alpha M (CD11b) promoter; Arabic numerals next to the promoter name correspond to an animal identification number) across murine cells of the lymphoid lineage (e.g., B cells, CD4+ T cells, and CD8+ T cells) and murine cells of the myeloid lineage (e.g., monocytes, granulocytes, and microglial cells). Heat map showing GFP expression in lymphoid and myeloid cells of the bone marrow in mice driven by different promoters (FIG. 5A). As can be seen by the darker shading in the lower-right quadrant of the heat map (corresponding to rows MND-1 through CD11b-5 and the monocyte and granulocyte columns), MND and CD11B promoter-driven GFP expression, but not PGK-1 or EFS-1 promoter-driven GFP expression, is higher in murine bone marrow macrophages and granulocytes as compared to bone marrow B cells, CD4+ T cells, and CD8+ T cells. Heat map showing GFP expression in blood lymphoid and myeloid cells in mice driven by different promoters (FIG. 5B). As can be seen by the darker shading in the lower-right quadrant of the heat map (corresponding to rows MND-1 through CD11b-5 and the macrophages and granulocyte columns, MND and CD11b promoter-driven GFP expression, but not PGK-1 or EFS1 promoter-driven GFP expression, is higher in murine bone marrow macrophages and granulocytes as compared to blood B cells, CD4+ T cells, and CD8+ T cells. Heat map showing GFP expression in brain microglial cells in mice driven by different promoters (FIG. 5C). As can be seen by the darker shading in the lower half of the heat map (corresponding to rows MND-1 through CD11b-5), MND and CD11b promoter-driven GFP expression is higher than GFP expression controlled by the PGK-1 and EFS1 promoters in brain microglia. These findings indicate that MND and CD11b promoters can be used to selectively drive transgene expression in cells of the myeloid lineage as compared to cells of the lymphoid lineage.

### DEFINITIONS

As used herein, the terms “ablate,” “ablating,” “ablation,” and the like refer to the depletion of one or more cells in a population of cells in vivo or ex vivo. In some embodiments of the present disclosure, it may be desirable to ablate endogenous cells within a subject (e.g., a subject undergoing treatment for a disease described herein, such as an NCD (e.g., Alzheimer's disease (AD), Nasu-Hakola disease (also known as polycystic lipomembranous osteodysplasia with sclerosing leukoencecphalopathy (PLOSL), frontotemporal lobar degeneration (FTLD), or Parkinson disease (PD)) before administering a therapeutic population of cells (e.g., pluripotent cells, embryonic stem cells (ESCs), induced pluripotent stem cells (iPSCs), multipotent cells, CD34+ cells, hematopoietic stem cells (HSCs), myeloid progenitor cells (MPCs), blood line progenitor cells (BLPCs), monocytes, macrophages, microglial progenitor cells, or microglia) to the subject. This can be beneficial, for example, in order to provide the newly-administered cells with an environment within which the cells may engraft. Ablation of a population of cells can be performed in a manner that selectively targets a specific cell type, for example, using antibody-drug conjugates that bind to an antigen expressed on the target cell and subsequently engender the killing of the target cell. Additionally or alternatively, ablation may be performed in a non-specific manner using cytotoxins that do not localize to a particular cell type, but are instead capable of exerting their cytotoxic effects on a variety of different cells. Exemplary agents that may be used to ablate a population of endogenous cells in a subject, such as a population of endogenous microglia or microglial precursor cells in a subject undergoing therapy, e.g., for the treatment of an NCD, are busulfan, PLX3397, PLX647, PLX5622, treosulfan, clodronate liposomes, and combinations thereof. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

As used herein, “administration” refers to providing or giving a subject a therapeutic agent (e.g., cells described herein) that includes a transgene (e.g., a transgene capable of expression in macrophages or microglia) encoding one or more triggering receptor expressed on myeloid cells two (TREM2) proteins, by any effective route. Exemplary routes of administration are described herein and below (e.g. intracerebroventricular (ICV) injection, intrathecal (IT) injection, intraparenchymal (IP) injection, intravenous (IV) injection, and stereotactic injection).

As used herein, “allogeneic” means cells, tissue, DNA, or factors taken or derived from a different subject of the same species. For example, in the context of transduced, TREM2-expressing cells that are administered to a subject for the treatment of an NCD, allogeneic cells may be cells that are obtained from a subject that is not the subject and are then transduced or transfected with a vector that directs the expression of TREM2. The phrase “directs expression” refers to the polynucleotide containing a sequence that encodes the molecule to be expressed. The polynucleotide may contain additional sequence that enhances expression of the molecule in question.

As used herein, “Alzheimer's disease” and “AD” refer to a late-onset neurodegenerative disorder presenting as cognitive decline, insidious loss of short- and long-term memory, attention deficits, language-specific problems, disorientation, impulse control, social withdrawal, anhedonia, and other symptoms. Brain tissue of AD patients exhibits neuropathological features such as extracellular aggregates of amyloid-3 protein and neurofibrillary tangles of hyperphosphorylated microtubule-associated tau proteins. Accumulation of these aggregates is associated with neuronal loss and atrophy in a number of brain regions including the frontal, temporal, and parietal lobes of the cerebral cortex as well as subcortical structures like the basal forebrain cholinergic system and the locus coeruleus within the brainstem. AD is also associated with increased neuroinflammation characterized by reactive gliosis and elevated levels of pro-inflammatory cytokines.

As used herein, “autologous” refers to cells, tissue, DNA, or factors taken or derived from an individual's own tissues, cells, or DNA. For example, in the context of transduced, TREM2-expressing cells that are administered to a subject for the treatment of an NCD, the autologous cells may be cells obtained from the subject that are then transduced or transfected with a vector that directs the expression of TREM2.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild type ApoE protein, such as proteins having at least 85% identity (e.g., at least 86%, 86%, 87%, 88%, 89%, 90%, 95%, 96%, 97%, 98%, 99%, or 99.9% identity, or more) to the amino acid sequence of wild type ApoE, which is set forth in SEQ ID NO. 13.

As used herein, the term “blood lineage progenitor cell” or “BLPC” refers to any cell (e.g., a mammalian cell) capable of differentiating into one or more (e.g., 2, 3, 4, 5 or more) types of hematopoietic (i.e., blood) cells. A BLPC may differentiate into erythrocytes, leukocytes (e.g., such as granulocytes (e.g., basophils, eosinophils, neutrophils, and mast cells) or agranulocytes (e.g., lymphocytes and monocytes)), or thrombocytes. A BLPC may also include a differentiated blood cell (e.g., a monocyte) that can further differentiate into another blood cell type (e.g., a macrophage).

As used herein, the term “CD11b promoter” refers to the native promoter of a CD11b gene. A CD11b promoter may have, for example, the nucleic acid sequence of SEQ ID NO. 17 or may be a variant thereof having at least 85% (e.g., at least 86%, 90%, 95%, 96%, 97%, 98%, 99%, or more) sequence identity to the nucleic acid sequence of SEQ ID NO. 17. According to the disclosed methods and compositions, the CD11b promoter is suitable for incorporation into a transgene expression construct (e.g., a plasmid or viral vector; e.g., an expression construct containing a polynucleotide having the nucleic acid sequence of SEQ ID NO. 18 or a variant thereof having at least 85% (e.g., at least 86%, 90%, 95%, 96%, 97%, 98%, 99%, or more) sequence identity to the nucleic acid sequence of SEQ ID NO. 18) for driving expression of a TREM2 transgene in one or more target cell types. The CD11b promoter was shown by the inventors of the present disclosure to drive heterologous transgene expression selectively in cells of the myeloid lineage (e.g., monocytes, granulocytes, and microglia), as compared to lymphoid cells such as B-cells and T-cells. Therefore, the CD11b promoter may be used to selectively drive therapeutic transgene expression in myeloid cells.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cistron” refers to a segment of a DNA or RNA sequence encoding a single protein or polypeptide product.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15:8125-8148, incorporated herein by reference in its entirety. Multiple stop codons can be incorporated.

As used herein, the term “cognitive test” refers to a test that can be performed by a skilled practitioner in order to assess the cognitive capabilities of humans and other animals. A cognitive test may be used to assess inductive reasoning skills, intelligence quotient, cognitive development, memory, knowledge organization, metacognition, thought, mental chronometry. A cognitive test may be used to assess the performance of a subject across several cognitive domains, including, but not limited to executive function, learning and memory, language, perceptual-motor function, and social cognition. Examples of cognitive tests include, but are not limited to Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). A skilled practitioner will recognize that other cognitive tests well-known in the art may also be used to assess cognitive function in a subject.

As used herein, the term “complex attention” refers to a cognitive function that describes a subject's (e.g., a human subject's) ability to maintain information in their mind for a short time and to perform an operation on that information (e.g., mental arithmetic). Impairment in complex attention may result in difficulty with focusing on conversations, difficulty filtering out unwanted information, problems with prospective memory (e.g., remembering to remember something later on), and inefficient memory for new information.

As used herein, the terms “condition” and “conditioning” refer to processes by which a subject is prepared for receipt of a transplant containing cells (e.g., pluripotent cells, ESCs, iPSCs, multipotent cells, CD34+ cells, HSCs, MPCs, BLPCs, monocytes, macrophages, microglial progenitor cells, or microglia). Such procedures promote the engraftment of a cell transplant, for example, by selectively depleting endogenous microglia or HSCs, thereby creating a vacancy filled by an exogenous cell transplant.

According to the methods described herein, a subject may be conditioned for cell transplant therapy by administration to the subject of one or more agents capable of ablating endogenous microglia and/or hematopoietic stem or progenitor cells (e.g., busulfan, treosulfan, PLX3397, PLX647, PLX5622, and clodronate liposomes), radiation therapy, or a combination thereof. Conditioning may be myeloablative or non-myeloablative. Other cell-ablating agents and methods well known in the art (e.g., antibody-drug conjugates) may also be used.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both neutral and | large |
| | | | | cationic forms | |
| | | | | in equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenyl- | Phe | F | nonpolar | neutral | large |
| alanine | | | | | |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the phrase “delirium or other mental disorder” refers to a condition such as delirium (i.e., a syndrome encompassing impaired attention, consciousness, and cognition that develops over a short period of time (e.g., hours to days)) or another disorder of the mind (e.g., schizophrenia, bipolar disorder, and major depression) that is distinct from a neurocognitive disorder and does not exhibit cognitive impairment as a core symptom. For example, a condition such as delirium or another mental disorder may differ from an NCD in that cognitive impairment may by a symptom that is associated with the disease but is not a central feature of said disease. Delirium or another mental disorder may differ from an NCD with respect to time to onset (e.g., hours to days in delirium versus months to years for an NCD), etiology (e.g., substance-induced delirium), symptom length (e.g., delirium may last hours to days whereas an NCD can last for years), and resolution (e.g., delirium may resolve completely, whereas an NCD does not resolve in most cases).

As used herein, the term “disrupt”, with respect to a gene, refers to preventing the formation of a functional gene product. A gene product is functional if it fulfills its normal (wild type) functions. Disruption of the gene prevents expression of a functional factor encoded by the gene and contains an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods for genetically modifying cells so as to disrupt the expression of one or more genes are detailed in U.S. Pat. Nos. 8,518,701; 9,499,808; and US 2012/0222143, the disclosures of each of which are incorporated herein by reference in their entirety (in case of conflict, the instant specification is controlling).

As used herein, the terms “effective amount,” “therapeutically effective amount,” and a “sufficient amount” of composition, vector construct, viral vector, or cell described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating an NCD (e.g., AD, PLOSL, FTLD, or PD), it is an amount of the composition, vector construct, viral vector, or cell sufficient to achieve a treatment response as compared to the response obtained without administration of the composition, vector construct, viral vector, or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be determined by one skilled in the art. Also, as used herein, a “therapeutically effective amount” of a composition, vector construct, viral vector, or cell of the present disclosure is an amount which results in a beneficial or desired result in a subject as compared to a control. As defined herein, a therapeutically effective amount of a composition, vector construct, viral vector, or cell of the present disclosure may be readily determined by one of ordinary skill by methods known in the art. Dosage regime may be adjusted to provide the optimum therapeutic response.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “engraft” and “engraftment” refer to the process by which hematopoietic stem cells and progenitor cells, whether such cells are produced endogenously within the body or transplanted using any of the administration methods described herein (e.g. intravenous injection, intracerebroventricular injection, intraosseous injection, and/or bone marrow transplant), repopulate a tissue. The term encompasses all events surrounding or leading up to engraftment, such as tissue homing of cells and colonization of cells within the tissue of interest.

As used herein, the term “executive function” refers to a set of cognitive functions that facilitate cognitive control of behavior in a subject (e.g., a human). Executive function encompasses, e.g., selection and monitoring goal-directed behaviors, attentional control, cognitive inhibition, inhibitory control, working memory, and cognitive flexibility. An individual normally acquires or perfects executive functions across the lifespan, although this process may be derailed by the development of an NCD in the subject, which may adversely impact executive function.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art) in a sample obtained from the subject.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the terms “frontotemporal lobar degeneration” and “FTLD” refer to a complex clinical syndrome characterized by degeneration of brain tissue within the frontal and temporal lobes of the cerebral cortex. The terms “frontotemporal lobar degeneration” and “FTLD” may refer to any one of three clinically distinct variants of FTLD including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe. Additionally, BVFTD has a strong association with amyotrophic lateral sclerosis; 2) semantic dementia (SD) is characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; 3) progressive nonfluent aphasia (PNA) is characterized by motor deficits in speech production, reduced language expression, and pronounced degeneration of the perisylvian cortex. Histopathological profiles of FTLD patients generally fall into one of three broad phenotypes including those that exhibit aggregation and deposition of (i) microtubule-associated tau protein inclusions; (ii) tau-negative, ubiquitin and TAR DNA-binding protein 43 (TDP-43)-positive protein inclusions, or (iii) ubiquitin and fused in sarcoma (FUS)-positive protein inclusions. For a comprehensive description of the clinical presentation and histopathology of FTLD, see Rabinovici and Miller, CNS Drugs 24(5): 375-398 (2010), the disclosure of which is herein incorporated by reference in its entirety.

As used herein, the term “functional ectodomain cleavage site” as it pertains to the TREM2 ectodomain cleavage site refers to amino acid residues within the full-length TREM2 peptide that undergo proteolytic cleavage by extracellular proteases (e.g., disintegrin and metalloprotease family) ectodomain to produce soluble TREM2 as well as the TREM2 C-terminal fragment. The TREM2 ectodomain cleavage site may be rendered non-functional as a result of, for example, a mutation in the TREM2 gene that alters the amino acid sequence within the ectodomain cleavage site or affects the tertiary protein structure in such a way as to sterically protect the ectodomain cleavage site from proteolytic cleavage.

As used herein, the term “functional intramembrane cleavage site” as it pertains to the TREM2 C-terminal fragment intramembrane cleavage site refers to amino acid residues within the TREM2 C-terminal fragment that undergo proteolytic cleavage by the γ-secretase complex to produce the TREM2 intracellular domain and TREM2-A p-like peptide. The TREM2 C-terminal fragment intramembrane cleavage site may be rendered non-functional as a result of, for example, a mutation in the TREM2 gene that alters the amino acid sequence within the intramembrane cleavage site or affects the tertiary protein structure in such a way as to sterically protect the intramembrane cleavage site from proteolytic cleavage.

As used herein, the term “functional potential” as it pertains to a stem cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., natural killer (NK) cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the term “general population” refers to an entire population of individuals having a particular characteristic of interest (e.g., age, medical history, education, socioeconomic status, or lifestyle, among others). Alternatively, the term “general population” may refer to a subset of the entire population of individuals having a particular characteristic of interest, such as, e.g., a random sample having a defined sample size. According to the methods disclosed herein, the general population may serve as a practical referent (e.g., a reference population) to which a measured variable can be compared. For example, a subject diagnosed with an NCD may have their cognition assessed using a cognitive test disclosed herein and the score obtained by the subject on the test may be compared against performance of individuals in the general population (e.g., the entire general population or a random sample of the general population) on the same test. The size of the random sample of the general population may be determined by a skilled practitioner using methods well-known in the art. For example, a skilled practitioner may perform a power analysis prior to collecting data (e.g., prior to conducting a cognitive test on a subject) to determine the smallest sample that is needed to detect a statistically significant effect with a desired level of confidence.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the phrase “independence or normal daily functioning” refers to the ability of a subject to successfully perform everyday activities without assistance from a caretaker or a social worker. Non-limiting examples of activities that enable an individual to independently carry out daily functions include, e.g., social, occupational, or academic functioning, personal hygiene, grooming, dressing, toilet hygiene, functional mobility (e.g., ability to walk, get in and out of bed), and self-feeding. A subject diagnosed with a major NCD, may have difficulty independently performing normal daily functions, whereas a subject diagnosed with mild NCD may not have difficulty independently performing daily tasks.

As used herein, the terms “induced pluripotent stem cell,” “iPS cell,” and “iPSC” refer to a pluripotent stem cell that can be derived directly from a differentiated somatic cell. Human PS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “IRES” refers to an internal ribosome entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the term “language” refers to a cognitive ability of a subject to learn and use systems of complex communication, or to describe the rules that govern these systems, or the collection of utterances that may be generated from such rules. Language ability may be impaired in a subject with an NCD if the subject exhibits, e.g., limited vocabulary, inability to produce complex grammar, frequent lexical errors, or aphasia, among others.

As used herein, the phrase “learning and memory” refer to a cognitive ability that encompasses the acquisition of skills or knowledge and expression of acquired skills or knowledge (e.g., learning to say a new word and uttering the new word, respectively). “Learning and memory” may refer to two independent processes of 1) acquiring new skills or knowledge (i.e., learning); and 2) processing, storing, and recalling the learned skill or knowledge (i.e., memory), which may differ by timescales (learning is generally slower and more effortful than recalling a memory or performing a learned skill) and neurobiological basis. A subject diagnosed with an NCD may have impaired learning and memory relative to a healthy subject.

As used herein, the term “leukodystrophy” refers to a set of predominately inherited disorders that feature degeneration of the white matter in the brain, which may result from defects in the myelin sheath that insulates neuronal axons. Leukodystrophies generally present around infancy and early childhood and may be characterized by hyperirritability, hypersensitivity to the environment, muscle rigidity, backwards-bent head, decrease or loss of hearing and vision, and epilepsy. Non-limiting examples of leukodystrophies include Nasu-Hakola disease, metachromatic leukodystrophy, Krabbe disease, X-linked adrenoleukodystrophy, Canavan disease, and Alexander disease.

As used herein, the term “lymphoid cell” refers to white blood cells of the lymphoid lineage that are derived from a common lymphoid progenitor cell. Lymphoid cells are generally part of the adaptive immunity arm of the immune system and include cells such as NK cells, B-cells, and T-cells. As used herein, lymphoid cells refers to a population of cells that cannot differentiate into cells of the myeloid lineage (e.g., MPCs, erythrocytes, mast cells, megakaryocytes, thrombocytes, myeloblasts, basophils, neutrophils, eosinophils, monocytes, and macrophages).

As used herein, the term “macrophage” refers to a type of white blood cell that engulfs and digests cellular debris, foreign substances, microbes, cancer cells, and anything else that does not have 15 the types of proteins specific to healthy body cells on its surface in a process called phagocytosis. Macrophages are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also 20 help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines.

As used herein, the terms “microglia” or “microglial cell” refer to a type of neuroglial cell found in the brain and spinal cord that function as resident macrophage cells and the principal line of immune defense in the central nervous system. Primary functions of microglial cells include immune surveillance, phagocytosis, extracellular signaling (e.g., production and release of cytokines, chemokines, prostaglandins, and reactive oxygen species), antigen presentation, and promotion of tissue repair and regeneration.

As used herein, the term “microglial progenitor cell” refers to a precursor cell that gives rise to microglial cells. Microglial precursor cells originate in the yolk sac during a limited period of embryonic development, infiltrate the brain mesenchyme, and perpetually renew themselves throughout life.

As used herein, the term “miRNA targeting sequence” refers to a nucleotide sequence located in the 3′-UTR of a target mRNA molecule which is complementary to a specific miRNA molecule (e.g. miR-126) such that they may hybridize and promote RNA-induced silencing complex-dependent and Dicer-dependent mRNA destabilization and/or cleavage, thereby preventing the expression of an mRNA transcript.

As used herein, the term “MND promoter” refers to a synthetic, constitutively active promoter derived from a myeloproliferative sarcoma virus (MSV). An MND promoter contains an MSV enhancer, a U3 region of a Maloney Murine Leukemia Virus, a deletion of a negative control region, and a substitution of a dl587rev primer binding site. An MND promoter may have, for example, the nucleic acid sequence of SEQ ID NO. 14 or may be a variant thereof having at least 85% (e.g., at least 86%, 90%, 95%, 96%, 97%, 98%, 99%, or more) sequence identity to the nucleic acid sequence of SEQ ID NO. 14 or SEQ ID NO. 16. According to the disclosed methods and compositions, the MND promoter is suitable for incorporation into a transgene expression construct (e.g., a plasmid or viral vector; e.g., an expression construct containing a polynucleotide having the nucleic acid sequence of SEQ ID NO. 15 or a variant thereof having at least 85% (e.g., at least 86%, 90%, 95%, 96%, 97%, 98%, 99%, or more) sequence identity to the nucleic acid sequence of SEQ ID NO. 15) for driving expression of a TREM2 transgene in one or more target cell types. The MND promoter was shown by the inventors of the present disclosure to drive heterologous TREM2 transgene expression selectively in peripheral blood-derived macrophages, as compared to lymphoid cells such as B-cells and T-cells. Therefore, the MND promoter may be used to selectively drive TREM2 transgene expression in myeloid cells (e.g., macrophages, among others).

As used herein, the term “monocistronic” refers to an RNA or DNA construct that contains the coding sequence for a single protein or polypeptide product.

As used herein, the term “monocyte” refers to a type of white blood cell (i.e., a leukocyte) that is capable of differentiating into macrophages and myeloid lineage dendritic cells. Monocytes constitute an important component of the vertebrate adaptive immune response. Three different types of monocytes are known to exist, including classical monocytes characterized by strong expression of the CD14 cell surface receptor and no CD16 expression (i.e., CD14++ CD16−), non-classical monocytes exhibiting low levels of CD14 expression and co-expression of C16 (CD14+ CD16+), and intermediate monocytes exhibiting high levels of CD14 expression and low levels of C16 expression (CD14++ CD16+). Monocytes perform a variety of functions that serve the immune system, including phagocytosis, antigen presentation, and cytokine secretion.

As used herein, the term “multipotent cell” refers to a cell that possesses the ability to develop into multiple (e.g., 2, 3, 4, 5, or more) but not all differentiated cell types. Non-limiting examples of multipotent cells include cells of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of multipotent cells are CD34+ cells.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion. The nomenclature for describing mutations and sequence variations uses the format “reference sequence.code,” wherein the reference sequence may be “c,” designating a coding DNA and the code may contain symbols including “>,” designating a single nucleotide substitution, “del,” designating a deletion, or may contain “a+b” in reference to substitutions occurring within an intron, wherein x denotes a number corresponding to a nucleotide within the coding DNA sequence (e.g., a nucleotide within an exon of a coding DNA sequence) and y corresponds to the number of nucleotides 3′ relative to x. For example, the TREM2 mutant associated with a substitution described as c.482+2T>C has a T to C substitution 2 nucleotides 3′ relative to the nucleotide in position 482 of the coding DNA sequence. Mutations may also result in a substitution of a single amino acid within the peptide chain. The nomenclature for describing mutations resulting amino acid substitutions uses the format “p.AnB,” where “p” designates the variation at the level of the protein, “A” designates the amino acid found in the wild type variant of the protein, “n” designates the number of the amino acid within the peptide chain, and “B” designates the new amino acid that resulted from the substitution. For example, a p.R47H variant of the TREM2 gene corresponds to a change in the protein at amino acid 47 where an arginine is substituted for histidine.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the term “myeloid cells” refers to blood cells derived from the bone marrow that belong to the myeloid cell lineage and arise from common myeloid progenitor cells that gives rise to granulocytes, monocytes, erythrocytes, and platelets. Non-limiting examples of myeloid cells include MPCs, erythrocytes, mast cells, megakaryocytes, thrombocytes, myeloblasts, basophils, neutrophils, eosinophils, monocytes, and macrophages. As used herein, myeloid cells refers specifically to a group of myeloid lineage cells that are not capable if differentiating into cells of the lymphoid lineage (e.g., NK cells, T-cells, B-cells, or plasma cells).

As used herein, “Nasu-Hakola disease” and “PLOSL” refer to a neurodegenerative disorder characterized by the presence of white matter degeneration, axonal spheroids, and cystic bone lesions in the upper and lower extremities. PLOSL patients exhibit early onset dementia as well as recurrent bone fractures. PLOSL generally proceeds through four distinct stages including the latent stage during infancy, followed by the osseous stage in adolescence when patients may experience polyarthralgias in hands, wrists, ankles, and feet. The osseous stage is followed by the early neurological stage during which patients may exhibit profound personality changes, progressive memory deficits, and epileptic seizures. The late neurological stage of PLOSL patients presents with profound dementia and motor incapacitation. Histopathological hallmarks of PLOSL include demyelination, loss of axons, emergence of axonal spheroids, fibrillary gliosis, and accumulation of lipid granules around blood vessels and within nervous tissue parenchyma. PLOSL patients also exhibit accumulation of lipid-laden macrophages and free fatty acids in the brain, along with vascular abnormalities within frontal and temporal cortical regions. For a comprehensive summary of the clinical, pathological, and cellular aspects of PLOSL, see Bianchin et al., Cellular and Molecular Neurobiology 24:1-24 (2004).

As used herein, the terms “neurocognitive disorder” or “NCD” refer to a set of clinical disorders or syndromes in which the primary clinical deficit is cognitive function, such as a deficit in, e.g., complex attention, executive function, learning and memory, language, perceptual-motor function, and social cognition. NCD is characterized as an acquired condition, rather than a developmental one. For example, an NCD is a condition in which disrupted cognition was not evident since birth or very early life, therefore requiring that cognitive function in NCD declined from a previously acquired level. NCD is distinguished from other disorders in which patients present with cognitive impairment in that NCD includes only disorders in which the core deficits are cognitive. NCD may be “major NCD” or “mild NCD.” Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major and mild NCD may also be differentiated on the basis of quantitative cognitive testing across any one of the specific cognitive functions described above. For example, major NCD can be characterized by a score obtained on a cognitive test by a subject identified as having or at risk of developing NCD that is more than two standard deviations away from the mean score of a reference population (e.g., the mean score of a general population) or a score that is in the third percentile of the distribution of scores of the reference population. Mild NCD can be characterized by a score obtained on a cognitive test by a subject identified as having or at risk of developing NCD that is between one to two standard deviations away from the mean score of a reference population or a score that is between the 3^{rd} and 16^{th} percentile of the distribution of scores of the reference population. Non-limiting examples of cognitive tests that can be used to categorize an NCD patient as having either major or mild NCD include AD8, AWV, GPCOG, HRA, MIS, MMSE, MoCA, SLUMS, and Short IQCODE. Furthermore, NCD includes syndrome subtypes that designate the particular etiological origin of the NCD, such as, e.g., AD or PLOSL. As used herein, the terms “NCD due to Alzheimer's disease” and “NCD due to a leukodystrophy” correspond to NCD caused by AD and leukodystrophy (e.g., PLOSL), respectively.

As used herein, the term “non-myeloablative” or “myelosuppressive” refers to a conditioning regiment that does not eliminate substantially all hematopoietic cells of host origin.

As used herein, the terms “Parkinson's disease” or “PD” refer to a progressive neurodegenerative disorder that affects movement, and it is recognized as the second most common neurodegenerative disease after Alzheimer's disease. Common symptoms of PD include resting tremor, rigidity, and bradykinesia, and non-motor symptoms, such as depression, constipation, pain, sleep disorders, genitourinary problems, cognitive decline, and olfactory dysfunction, are also increasingly being associated with PD. A key feature of PD is the death of dopaminergic neurons in the substantia nigra pars compacta, and, for that reason, most current treatments for PD focus on increasing dopamine. Another well-known neuropathological hallmark of PD is the presence of Lewy bodies containing α-synuclein in brain regions affected by PD, which are thought to contribute to the disease.

As used herein, the term “perceptual-motor function” refers to a cognitive ability that enables a subject (e.g., a human) to interact with their environment using sensory and motor skills. Perceptual-motor functions encompass the coordination of sensory and motor skills to allow a person to execute movements in accord with the environmental context in which the subject is embedded. Perceptual-motor functions may include, but are not limited to body awareness, spatial awareness, directional awareness, and chronometry. Particular manifestations of perceptual-motor function may include throwing, catching, kicking, jumping, swinging, cutting, lacing, hammering, buttoning, pouring, naming, pointing, identifying, moving, performing tasks using body parts, exploring, locating, comparing, walking, running, rolling, stationing, balancing, clapping, hitting or tracking a moving object, matching visual and motor responses, among others. A subject diagnosed with an NCD may exhibit impaired perceptual-motor function relative to a healthy subject.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs and iPSCs.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polycistronic” refers to an RNA or DNA construct that contains the coding sequence for at least two protein or polypeptide products.

As used herein, the term “promoter” refers to a recognition site on DNA that is bound by an RNA polymerase. The polymerase drives transcription of the transgene. Exemplary promoters suitable for use with the compositions and methods described herein include synthetic promoters, which are regulatory nucleic acids that do not occur naturally in biological systems. Synthetic promoters contain parts of naturally occurring promoters combined with nucleic acids that do not occur in nature and can be optimized to express recombinant DNA using a variety of transgenes, vectors, and target cell types. An exemplary synthetic promoter suitable for use with the presently disclosed methods and compositions include an MND promoter (e.g., SEQ ID NO. 14 or SEQ ID NO. 16), which are described, for example, in Halene et al. Blood 94:3349-57 (1999), the disclosure of which is incorporated herein by reference as it pertains to the MND promoter.

As used herein, the term “high expression constitutive promoter” refers to a promoter that drives expression of a transgene (e.g., a transgene encoding TREM2, such as a transgene having at least 85% (e.g., at least 86%, 90%, 95%, 96%, 97%, 98%, 99%, or more) sequence identity to the nucleic acid sequence of any one of SEQ ID NOs: 4-12) in cells of the myeloid lineage or progenitors thereof (e.g., CD34+ cells (e.g., HSCs or MPCs), BLPCs (e.g., monocytes), microglial progenitor cells, macrophages, or microglia) transformed with a vector (e.g., plasmid or viral vector) encoding such promoter operably linked to the TREM2 transgene at a level that is at least 50% higher than expression of the transgene in cells of the lymphoid lineage, such as, e.g., T-cells or B-cells, transformed with the same amount (e.g., titer) of an equivalent vector encoding the TREM2 transgene operably linked to the high expression constitutive promoter. In some embodiments, a high expression constitutive promoter can express the transgene in cells of the myeloid lineage at a level that is, e.g., at least 50%, 55%, 60%, 65%, 70%, 75%, 80%, 85%, 90%, 95%, 96%, 97%, 98%, 99%, 100%, 150%, 200%, 250%, 300%, 350%, 400%, 450%, 500% or higher than expression in cells of the lymphoid lineage. In some embodiments, the higher level of TREM2 expression in a myeloid cell as compared to a lymphoid refers to a higher level of TREM2 protein expression within a single myeloid cell as compared to a single lymphoid cell. In some embodiments, the higher level of TREM2 expression in myeloid cells as compared to lymphoid cells refers to a higher number of TREM2-expressing (i.e., TREM2-positive) myeloid cells within an organ, tissue, or bodily fluid (e.g., peripheral blood) as compared to a number of TREM2-positive lymphoid cells in the same organ, tissue, or bodily fluid. In some embodiments, the high expression constitutive promoter is a Sarcoma Virus Enhancer, Negative Control Region Deleted, dl587rev Primer-Binding Site Substituted (MND) promoter, such as an MND promoter having at least 85% (e.g., at least 86%, 90%, 95%, 96%, 97%, 98%, 99%, or more) sequence identity to the nucleic acid sequence of SEQ ID NO. 14 or SEQ ID NO. 16. In some embodiments, the high expression constitutive promoter is an integrin subunit alpha M (CD11b) promoter, such as a CD11b promoter having at least 85% (e.g., at least 86%, 90%, 95%, 96%, 97%, 98%, 99%, or more) sequence identity to the nucleic acid sequence of SEQ ID NO. 17.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full-length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a potent “receptor-binding peptide (Rb) derived from ApoE” has the ability to translocate proteins across the BBB into the brain when engineered as fusion proteins. This method can therefore function to selectively open the BBB for therapeutic agents (e.g., soluble TREM2) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Exemplary Rb domains can be found in the N-terminus of ApoE. For example, Rb domains useful in conjunction with the compositions and methods described herein are polypeptides having the amino acid sequence of residues 1 to 191 of SEQ ID NO. 13, residues 25 to 185 of SEQ ID NO. 13, residues 50 to 180 of SEQ ID NO. 13, residues 75 to 175 of SEQ ID NO. 13, residues 100 to 170 of SEQ ID NO. 13, or residues 125 to 165 of SEQ ID NO. 13, as well as variants thereof, such as polypeptides having at least 85% sequence identity (e.g., at least 86%, 86%, 87%, 88%, 89%, 90%, 95%, 96%, 97%, 98%, 99%, or greater, sequence identity) with respect to any of these sequences. An exemplary Rb domain is the region of ApoE having the amino acid sequence of residues 159 to 167 of SEQ ID NO. 13.

As used herein, the term “regulatory sequence” includes promoters, enhancers and other expression control elements (e.g., polyadenylation signals) that control the transcription or translation of the antibody chain genes. Such regulatory sequences are described, for example, in Perdew et al., Regulation of Gene Expression (Humana Press, New York, NY, (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “signal peptide” refers to a short (usually between 16-30 amino acids) peptide region that directs translocation of the translated protein from the cytoplasm of the host to the lipid membrane for anchoring. Such signal peptides are generally located at the amino terminus of the newly translated protein. In some embodiments, the signal peptide is linked to the amino terminus. Typically, signal peptides are cleaved during transit through the endoplasmic reticulum. Cleavage is not essential as long as the protein retains its desired activity. Exemplary signal peptide includes the TREM2 signal peptide.

As used herein, the term “social cognition” refers to a cognitive function that encompasses a set of skills that govern how subjects (e.g., humans) process, store, and apply information about other conspecific subjects (e.g., other humans) and social situations. Non-limiting examples of social cognition include, e.g., emotional responses to social stimuli, performance on theory of mind tasks, ability to recognize faces, impulse control in social contexts, and joint attention. A subject diagnosed with an NCD may exhibit impaired social cognition relative to a healthy subject.

As used herein, the term “splice variant” refers to a transcribed product (i.e. RNA) of a single gene that can be processed to produce different mRNA molecules as a result of alternative inclusion or exclusion of specific exons (e.g. exon skipping) within the precursor mRNA. Proteins produced from translation of specific splice variants may differ in their structure and biological activity.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., TREM2). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “triggering receptor expressed on myeloid cells two” and “TREM2” refer to the transmembrane glycoprotein belonging to the immunoglobulin variable domain receptor family. The gene is located on human chromosome 6p21.1. The terms “triggering receptor expressed on myeloid cells two” and “TREM2” also refer to variants of wild type TREM2 peptides and nucleic acids encoding the same, including splice variants resulting from alternative splicing of TREM2 primary transcripts, such as variant proteins having at least 85% sequence identity (e.g., 86%, 87%, 88%, 89%, 90%, 95%, 96%, 97%, 98%, 99%, or 99.9% identity, or more) to the amino acid sequence of a wild type TREM2 peptide (e.g., any one of SEQ ID NOS. 1-3) or polynucleotides having at least 85% sequence identity (e.g., 86%, 87%, 88%, 89%, 90%, 95%, 96%, 97%, 98%, 99%, or 99.9% identity, or more) to the nucleic acid sequence of a wild type TREM2 gene (e.g., any of the nucleic acid sequences selected from SEQ ID NOs. 4-6, 7, 9), provided that the TREM2 isoform encoded retains the therapeutic function of wild type TREM2. The terms “triggering receptor expressed on myeloid cells two” and “TREM2” may also refer to a TREM2 protein in which the natural signal peptide is present. Furthermore, the terms “triggering receptor expressed on myeloid cells two” and “TREM2” may refer to all products of TREM2 proteolytic cleavage including soluble TREM2 (sTREM2), the TREM2 C-terminal fragment (CTF), the TREM2 intracellular domain (TREM2-ICD), and TREM2-A p-like peptides (T23). TREM2 cleavage occurs once the mature polypeptide has been translocated to the membrane following posttranslational processing within the endoplasmic reticulum and is mediated by members of the disintegrin and metalloprotease (ADAM) family. The full-length TREM2 peptide is first cleaved at the ectodomain to produce an extracellular sTREM2 peptide and the transmembrane TREM2-CTF, the latter of which may be further cleaved by the γ-secretase complex to produce the cytoplasmic TREM2-ICD and the extracellular TREM-T23 peptides. The terms “triggering receptor expressed on myeloid cells two” and “TREM2” may refer to a TREM2 protein lacking a functional ectodomain cleavage site. The terms “triggering receptor expressed on myeloid cells two” and “TREM2” may also refer to a TREM2 protein lacking a functional intramembrane cleavage site within the TREM2-CTF. Additionally, the terms “triggering receptor expressed on myeloid cells two” and “TREM2” may refer to a “TREM2 fusion protein,” which is a protein in which the TREM2 is operably linked to another polypeptide, half-life-modifying agent, or therapeutic agent, such as an ApoE Rb domain (such as an Rb domain having the amino acid sequence of residues 25-185, 50-180, 75-175, 100-170, 125-160, or 130-150 of SEQ ID NO. 13). As used herein, “TREM2” may refer to the peptide or the gene encoding this protein, depending upon the context, as will be appreciated by one of skill in the art.

As used herein, subjects suffering from “triggering receptor expressed on myeloid cells two-associated AD” and “TREM2-associated AD or PLOSL” are those subjects that have been diagnosed as having AD or PLOSL and also contain a deleterious mutation in the TREM2 gene. Over 40 mutations have been reported in the human TREM2 gene, which have variable effects on downstream signaling, trafficking, ligand binding, and cell surface expression. TREM2 mutations are discussed in in Guerreiro et al., The New England Journal of Medicine 368:117-27 (2013); Jonsson et al., The New England Journal of Medicine 368:107-16 (2013); Ulrich et al., Neuron Review 94:237-48 (2017); and Xing et al., Research and Reports in Biochemistry, 5:89-100 (2015); the disclosures of which are incorporated herein by reference as they pertain to human TREM2 mutations in AD and PLOSL.

As used herein, the terms “subject” and “patient” refer to an animal (e.g., a mammal, such as a human). A subject to be treated according to the methods described herein may be one who has been diagnosed with an NCD, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a viral vector construct or a part thereof into a cell and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of TREM2 as described herein include plasmids that contain regulatory sequences, such as promoter and enhancer regions, which direct gene transcription. Other useful vectors for expression of TREM2 contain polynucleotides that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### DETAILED DESCRIPTION

Described herein are compositions and methods for the treatment of a neurocognitive disorder (NCD), such as, e.g., Alzheimer's disease (AD) or Nasu-Hakola Disease, also known as polycystic lipomembranous osteodysplasia with sclerosing leukoencephalopathy (PLOSL) in a subject (such as a mammalian subject, for example, a human). Using the compositions and methods described herein, one can treat an NCD (e.g., AD (e.g., triggering receptor expressed on myeloid cells two (TREM2)-associated AD), PLOSL (e.g., TREM2-associated PLOSL), frontotemporal lobar degeneration (FTLD; e.g., TREM2-associated FTLD), or Parkinson disease (PD; e.g., TREM2-associated PD)) in a subject (e.g., a human subject) by administering cells (e.g., pluripotent cells, embryonic stem cells (ESCs), induced pluripotent stem cells (iPSCs), multipotent cells, CD34+ cells, hematopoietic stem cells (HSCs), myeloid progenitor cells (MPCs), blood line progenitor cells (BLPCs), monocytes, macrophages, microglial progenitor cells, or microglia) containing a transgene encoding TREM2 (e.g., such as a transgene capable of expressing TREM2 in a macrophage or a microglial cell) operably linked to a high expression constitutive promoter, such as, e.g., an MND promoter or a CD11b promoter. For example, described herein are compositions containing cells that have been modified ex-vivo to express TREM2. The sections that follow describe the compositions and methods useful for the treatment of NCD in further detail.

### Neurocognitive Disorders

NCDs are defined as a collection of disorders that feature cognitive impairment as a core symptom and that show cognitive decline relative to a previously higher level of cognition (e.g., acquired impairment), rather than a developmental impairment. NCDs are broadly divided into major or mild syndromes (e.g., major NCD and mild NCD) based on the degree of impairment diagnosed in the subject. Furthermore, NCDs can be categorized on the basis of their etiological origin. For example, non-limiting examples of NCD may include NCD due to AD, NCD due to a leukodystrophy (e.g., PLOSL), vascular NCD, NCD with Lewy bodies, NCD due to Parkinson disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The compositions and methods disclosed herein are useful for the treatment of NCDs.

### Alzheimer's Disease

AD is an NCD characterized by progressive neuronal loss in the frontal, temporal, and parietal lobes of the cerebral cortex as well as subcortical structures like the basal forebrain cholinergic system and the locus coeruleus within the brainstem. The clinical presentation of AD is a progressive decline in a number of cognitive functions including short and long-term memory, spatial navigation, language fluency, impulse control, anhedonia, and social withdrawal. Neuronal atrophy in brains of AD patients is linked to accumulation of extracellular and intracellular protein inclusions. Aggregates of insoluble amyloid-β (Aβ) protein are often found in the extracellular space, while neurofibrillary tangles (NFTs) of hyperphosphorylated tau proteins are usually found in intracellular compartments of affected neurons. These neuropathologies are considered to be important in the etiology of AD.

The likelihood of developing AD is strongly affected by genetic factors. Known mutations in genes encoding amyloid precursor protein (APP), or the proteolytic enzymes that cleave APP like presenilin-1 (PSEN1) and presenilin-2 (PSEN2) have been established as risk factors for early-onset AD. These mutations are associated with an increased accumulation of the pathogenic Aβ₄₂ isoform that is the main constituent of Aβ deposits in the brain. Elevated risk for late-onset AD has been strongly linked to variations in the ε4 allele of apolipoprotein-E (APOE).

### Nasu-Hakola Disease

Nasu-Hakola Disease (PLOSL) is a rare autosomal recessive leukodystrophy characterized by the presence of white matter degeneration, loss of axons and myelin, presence of axonal spheroids, as well as cystic bone lesions in the distal extremities. Clinical manifestations of PLOSL patients include early onset dementia as well as recurrent bone fractures. Unlike AD, which largely affects the older patients, PLOSL may begin presenting symptoms during adolescence during the osseous stage when patients may experience polyarthralgias in hands, wrists, ankles, and feet. The osseous stage is followed by the early neurological stage during which patients may exhibit profound personality changes, progressive memory deficits, and generalized epileptic seizures. The late neurological stage of PLOSL patients corresponds to profound dementia, motor incapacitation, and ultimately death.

### Frontotemporal Lobar Degeneration

FTLD is a clinical syndrome characterized by progressive neurodegeneration in the frontal and temporal lobes of the cerebral cortex. The manifestation of FTLD is complex and heterogeneous, and may present as one of three clinically distinct variants including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe; 2) semantic dementia (SD), characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; or 3) progressive nonfluent aphasia (PNA); characterized by motor deficits in speech production, reduced language expression, and pronounced degeneration of the perisylvian cortex. Neuronal loss in brains of FTLD patients is associated with one of three distinct neuropathologies: 1) the presence of tau-positive neuronal and glial inclusions; 2) ubiquitin (ub)-positive and TAR DNA-binding protein 43 (TDP43)-positive, but tau-negative inclusions; or 3) ub and fused in sarcoma (FUS)-positive, but tau and TDP-43-negative inclusions. These neuropathologies are considered to be important in the etiology of FTLD.

Nearly half of FTLD patients have a first-degree family member with dementia, ALS, or Parkinson's disease, suggesting a strong genetic link to the cause of the disease. A number of mutations in chromosome 17q21 have been linked to FTLD presentation.

### Parkinson's Disease

PD is a progressive disorder that affects movement, and it is recognized as the second most common neurodegenerative disease after Alzheimer's disease. Common symptoms of PD include resting tremor, rigidity, and bradykinesia, and non-motor symptoms, such as depression, constipation, pain, sleep disorders, genitourinary problems, cognitive decline, and olfactory dysfunction, are also increasingly being associated with PD. A key feature of PD is the death of dopaminergic neurons in the substantia nigra pars compacta, and, for that reason, most current treatments for PD focus on increasing dopamine. Another well-known neuropathological hallmark of PD is the presence of Lewy bodies containing α-synuclein in brain regions affected by PD, which are thought to contribute to the disease.

PD is thought to result from a combination of genetic and environmental risk factors. There is no single gene responsible for all Parkinson's disease cases, and the vast majority of PD cases seem to be sporadic and not directly inherited. Mutations in the genes encoding parkin, PTEN-induced putative kinase 1 (PINK1), leucine-rich repeat kinase 2 (LRRK2), and Parkinsonism-associated deglycase (DJ-1) have been found to be associated with PD, but they represent only a small subset of the total number of PD cases. Occupational exposure to some pesticides and herbicides has also been proposed as a risk factor for PD. The synthetic neurotoxin MPTP can cause Parkinsonism, but its use is extremely rare.

### TREM2-Associated Alzheimer's Disease

Recent studies based on genome and exome sequencing have revealed TREM2 as a major risk factor in the development of NCD (e.g., AD, PLOSL, FTLD, or PD). Multiple variants in the TREM2 gene have since been linked to increased risk for AD, with the most common variant being the rs75932628 single nucleotide polymorphism, which results in a arginine to histidine substitution at amino acid 47 (R47H). This mutation is considered to potentially impact the ligand-binding properties of TREM2. Other TREM2 variants have been shown to result in protein truncation and/or mis-folding, disrupted trafficking, reduced cell-surface expression, and enhanced or reduced activation of downstream signaling pathways. TREM2 is a transmembrane protein expressed on mononuclear phagocytes (e.g. microglia, osteoclasts, and alveolar macrophages) and can be activated by lipids or lipoproteins on its extracellular domain. Lacking an intracellular signaling domain, TREM2 exerts its effects on multiple intracellular signaling pathways through interactions with a transmembrane adaptor protein DAP12. Furthermore, membrane-bound TREM2 can be cleaved by extracellular proteases to generate soluble TREM2 (sTREM2) and a transmembrane C-terminal fragment (TREM2-CTF), which may be further cleaved by the γ-secretase complex to produce a cytoplasmic TREM2 intracellular domain (TREM2-ICD) and an extracellular TREM2-A β-like (TREM2-T2β) peptide. TREM2 activity is thought to be important for a number of functions within the cell including control of phagocytosis, suppression of inflammatory signals, and cell survival. AD-associated and PLOSL-associated mutations in TREM2 have been associated with impaired or enhanced TREM2 activity, implicating the importance of TREM2 homeostasis in the etiology of AD and PLOSL. Furthermore, AD patients carrying the R47H TREM2 variant have reduced microglial recruitment to Aβ plaques, suggesting that altered TREM2 activity may impair the normal functioning of microglia. Similarly, PLOSL is associated with the histopathological presence of lipid-laden macrophages, suggesting immune dysregulation. Proteolytic processing of TREM2 may also be altered in AD. Levels of sTREM2 appear to be elevated in the cerebrospinal fluid of AD patients and show a correlation with levels of phosphorylated tau proteins in the brain. TREM2 involvement in NCD (e.g., AD and PLOSL) is discussed in depth in Ulrich et al., Neuron Reviews 94:237-48 and Xing et al., Research and Reports in Biochemistry 5:89-100 (2015), the disclosures of which are incorporated herein by reference as they pertain to human TREM2 signaling in AD.

Clinical management of NCDs has employed pharmacological and behavioral interventions to mitigate disease symptoms. For example, acetylcholinesterase inhibitors have been used to elevate acetylcholine levels in the brain as a means to ameliorate cognitive deficits of AD as this neurotransmitter is found to be depleted in AD patients. Additionally, atypical antipsychotics are commonly prescribed to AD patients for behavioral management. Similarly, anti-convulsive drugs have been administered to PLOSL patients in order to control the spontaneous emergence of epileptic seizures. This strategy, however, is targeted at ameliorating the symptoms of the disease without addressing its development and progression. Unlike these treatments, the compositions and methods described herein provide the benefit of treating a different biochemical phenomenon that can underlie the development of the NCD. As such, the compositions and methods described herein target the physiological cause of the disease, representing a potential curative therapy.

The compositions and methods described herein can be used to treat an NCD by administering cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding TREM2 (such as a transgene capable of expression in macrophages or microglial cells). These compositions and methods can be used to treat an NCD with any etiology, e.g., genetic mutation, environmental toxin, or sporadic. These compositions and methods can also be used to treat a subject with TREM2-associated AD or PLOSL. The compositions and methods described herein can be used to treat a subject with normal TREM2 activity, reduced TREM2 activity, and a subject whose TREM2 mutational status and/or TREM2 activity level is unknown. The compositions and methods described herein may also be administered as a preventative treatment to a subject at risk of developing an NCD, e.g., a subject with a TREM2 mutation, a subject with reduced TREM2 activity, or a subject with a mutation in one or more of the genes associated with an NCD.

TREM2-encoding constructs that may be used in conjunction with the compositions and methods described herein include polynucleotides that encode wild-type TREM2 (any one of the amino acid sequences which are shown as SEQ ID NOS. 1-3) or a variant thereof, such as a polynucleotide that encodes a protein having at least 85% sequence identity (e.g., at least 86%, 90%, 95%, 96%, 97%, 98%, 99%, or more, sequence identity) to any of the amino acid sequences of SEQ ID NOS. 1-3.

In some embodiments, the TREM2 has an amino acid sequence of SEQ ID NO. 1 or is a variant thereof having at least 85% (e.g. at least 86%, 90%, 95%, 96%, 97%, 98%, 99%, or more) sequence identity to the amino acid sequence of SEQ ID NO. 1. In some embodiments, the TREM2 has an amino acid sequence having at least 90% (e.g. at least 91%, 95%, 96%, 97%, 98%, 99%, or more) sequence identity to the amino acid sequence of SEQ ID NO. 1. In some embodiments, the TREM2 has an amino acid sequence having at least 95% (e.g. at least 96%, 97%, 98%, 99%, or more) sequence identity to the amino acid sequence of SEQ ID NO. 1. In some embodiments, the TREM2 has an amino acid sequence having at least 98% (e.g. at least 99% or more) sequence identity to the amino acid sequence of SEQ ID NO. 1. In some embodiments, the TREM2 has an amino acid sequence having at least 99% sequence identity to the amino acid sequence of SEQ ID NO. 1. In some embodiments, the TREM2 has the amino acid sequence of SEQ ID NO. 1.

In some embodiments, the TREM2 has an amino acid sequence of SEQ ID NO. 2 or is a variant thereof having at least 85% (e.g. at least 86%, 90%, 95%, 96%, 97%, 98%, 99%, or more) sequence identity to the amino acid sequence of SEQ ID NO. 2. In some embodiments, the TREM2 has an amino acid sequence having at least 90% (e.g. at least 91%, 95%, 96%, 97%, 98%, 99%, or more) sequence identity to the amino acid sequence of SEQ ID NO. 2. In some embodiments, the TREM2 has an amino acid sequence having at least 95% (e.g. at least 96%, 97%, 98%, 99%, or more) sequence identity to the amino acid sequence of SEQ ID NO. 2. In some embodiments, the TREM2 has an amino acid sequence having at least 98% (e.g. at least 99% or more) sequence identity to the amino acid sequence of SEQ ID NO. 2. In some embodiments, the TREM2 has an amino acid sequence having at least 99% sequence identity to the amino acid sequence of SEQ ID NO. 2. In some embodiments, the TREM2 has the amino acid sequence of SEQ ID NO. 2.

In some embodiments, the TREM2 has an amino acid sequence of SEQ ID NO. 3 or is a variant thereof having at least 85% (e.g. at least 86%, 90%, 95%, 96%, 97%, 98%, 99%, or more) sequence identity to the amino acid sequence of SEQ ID NO. 3. In some embodiments, the TREM2 has an amino acid sequence having at least 90% (e.g. at least 91%, 95%, 96%, 97%, 98%, 99%, or more) sequence identity to the amino acid sequence of SEQ ID NO. 3. In some embodiments, the TREM2 has an amino acid sequence having at least 95% (e.g. at least 96%, 97%, 98%, 99%, or more) sequence identity to the amino acid sequence of SEQ ID NO. 3. In some embodiments, the TREM2 has an amino acid sequence having at least 98% (e.g. at least 99% or more) sequence identity to the amino acid sequence of SEQ ID NO. 3. In some embodiments, the TREM2 has an amino acid sequence having at least 99% sequence identity to the amino acid sequence of SEQ ID NO. 3. In some embodiments, the TREM2 has the amino acid sequence of SEQ ID NO. 3.

In some embodiments, the transgene encoding TREM2 includes a TREM2 polynucleotide having the nucleic acid sequence of SEQ ID NO. 4 or a variant thereof having at least 85% (e.g. at least 86%, 90%, 95%, 96%, 97%, 98%, 99%, or more) sequence identity to the nucleic acid sequence of SEQ ID NO. 4. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 4. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 4. In some embodiments, the transgene encoding TREM2 includes a TREM2 polynucleotide having at least 98% (e.g. at least 99% or more) sequence identity to the nucleic acid sequence of SEQ ID NO. 4. In some embodiments, the transgene encoding TREM2 includes a TREM2 polynucleotide having at least 99% sequence identity to the nucleic acid sequence of SEQ ID NO. 4. In some embodiments, the transgene encoding TREM2 includes a TREM2 polynucleotide having the nucleic acid sequence of SEQ ID NO. 4.

In some embodiments, the transgene encoding TREM2 includes a TREM2 polynucleotide having the nucleic acid sequence of SEQ ID NO. 5 or a variant thereof having at least 85% (e.g. at least 86%, 90%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 5. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 5. In some embodiments, the transgene encoding TREM2 includes a TREM2 polynucleotide having at least 98% (e.g. at least 99% or more) sequence identity to the nucleic acid sequence of SEQ ID NO. 5. In some embodiments, the transgene encoding TREM2 includes a TREM2 polynucleotide having at least 99% sequence identity to the nucleic acid sequence of SEQ ID NO. 5. In some embodiments, the transgene encoding TREM2 includes a TREM2 polynucleotide having the nucleic acid sequence of SEQ ID NO. 5.

In some embodiments, the transgene encoding TREM2 includes a TREM2 polynucleotide having the nucleic acid sequence of SEQ ID NO. 6 or a variant thereof having at least 85% (e.g. at least 86%, 90%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 6. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 6. In some embodiments, the transgene encoding TREM2 includes a TREM2 polynucleotide having at least 98% (e.g. at least 99% or more) sequence identity to the nucleic acid sequence of SEQ ID NO. 6. In some embodiments, the transgene encoding TREM2 includes a TREM2 polynucleotide having at least 99% sequence identity to the nucleic acid sequence of SEQ ID NO. 6. In some embodiments, the transgene encoding TREM2 includes a TREM2 polynucleotide having the nucleic acid sequence of SEQ ID NO. 6.

In some embodiments, the transgene encoding TREM2 includes a TREM2 polynucleotide having the nucleic acid sequence of SEQ ID NO. 7 or a variant thereof having at least 85% (e.g. at least 86%, 90%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 7. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 7. In some embodiments, the transgene encoding TREM2 includes a TREM2 polynucleotide having at least 98% (e.g. at least 99% or more) sequence identity to the nucleic acid sequence of SEQ ID NO. 7. In some embodiments, the transgene encoding TREM2 includes a TREM2 polynucleotide having at least 99% sequence identity to the nucleic acid sequence of SEQ ID NO. 7. In some embodiments, the transgene encoding TREM2 includes a TREM2 polynucleotide having the nucleic acid sequence of SEQ ID NO. 7.

In some embodiments, the transgene encoding TREM2 includes a TREM2 polynucleotide having the nucleic acid sequence of SEQ ID NO. 9 or a variant thereof having at least 85% (e.g. at least 86%, 90%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 9. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 9. In some embodiments, the transgene encoding TREM2 includes a TREM2 polynucleotide having at least 98% (e.g. at least 99% or more) sequence identity to the nucleic acid sequence of SEQ ID NO. 9. In some embodiments, the transgene encoding TREM2 includes a TREM2 polynucleotide having at least 99% sequence identity to the nucleic acid sequence of SEQ ID NO. 9. In some embodiments, the transgene encoding TREM2 includes a TREM2 polynucleotide having the nucleic acid sequence of SEQ ID NO. 9.

In some embodiments, the transgene encoding TREM2 includes a TREM2 polynucleotide having the nucleic acid sequence of SEQ ID NO. 11 or a variant thereof having at least 85% (e.g. at least 86%, 90%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 90% (e.g. at least 91%, 95%, 96%, 97%, 98%, 99%, or more) sequence identity to the nucleic acid sequence of SEQ ID NO. 11. In some embodiments, the transgene encoding TREM2 includes a TREM2 polynucleotide having at least 95% (e.g. at least 96%, 97%, 98%, 99%, or more) sequence identity to the nucleic acid sequence of SEQ ID NO. 11. In some embodiments, the transgene encoding TREM2 includes a TREM2 polynucleotide having at least 98% (e.g. at least 99% or more) sequence identity to the nucleic acid sequence of SEQ ID NO. 11. In some embodiments, the transgene encoding TREM2 includes a TREM2 polynucleotide having at least 99% sequence identity to the nucleic acid sequence of SEQ ID NO. 11. In some embodiments, the transgene encoding TREM2 includes a TREM2 polynucleotide having the nucleic acid sequence of SEQ ID NO. 11.

In some embodiments, the transgene encoding TREM2 may be codon-optimized (e.g., any one of SEQ ID NO. 8, SEQ ID NO. 10, or SEQ ID NO. 12).

In some embodiments, the transgene encoding TREM2 includes a codon-optimized TREM2 polynucleotide of SEQ ID NO. 8 or a variant thereof having at least 85% (e.g. at least 86%, 90%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 95% (e.g. at least 96%, 97%, 98%, 99%, or more) sequence identity to the nucleic acid sequence of SEQ ID NO. 8. In some embodiments, the transgene encoding TREM2 includes a codon-optimized TREM2 polynucleotide having at least 98% (e.g. at least 99% or more) sequence identity to the nucleic acid sequence of SEQ ID NO. 8. In some embodiments, the transgene encoding TREM2 includes a codon-optimized TREM2 polynucleotide having at least 99% sequence identity to the nucleic acid sequence of SEQ ID NO. 8. In some embodiments, the transgene encoding TREM2 includes a codon-optimized TREM2 polynucleotide of SEQ ID NO. 8.

In some embodiments, the transgene encoding TREM2 includes a codon-optimized TREM2 polynucleotide of SEQ ID NO. 10 or a variant thereof having at least 85% (e.g. at least 86%, 90%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 95% (e.g. at least 96%, 97%, 98%, 99%, or more) sequence identity to the nucleic acid sequence of SEQ ID NO. 10. In some embodiments, the transgene encoding TREM2 includes a codon-optimized TREM2 polynucleotide having at least 98% (e.g. at least 99% or more) sequence identity to the nucleic acid sequence of SEQ ID NO. 10. In some embodiments, the transgene encoding TREM2 includes a codon-optimized TREM2 polynucleotide having at least 99% sequence identity to the nucleic acid sequence of SEQ ID NO. 10. In some embodiments, the transgene encoding TREM2 includes a codon-optimized TREM2 polynucleotide of SEQ ID NO. 10.

In some embodiments, the transgene encoding TREM2 includes a codon-optimized TREM2 polynucleotide of SEQ ID NO. 12 or a variant thereof having at least 85% (e.g. at least 86%, 90%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 90% (e.g. at least 91%, 95%, 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 95% (e.g. at least 96%, 97%, 98%, 99%, or more) sequence identity to the nucleic acid sequence of SEQ ID NO. 12. In some embodiments, the transgene encoding TREM2 includes a codon-optimized TREM2 polynucleotide having at least 98% (e.g. at least 99% or more) sequence identity to the nucleic acid sequence of SEQ ID NO. 12. In some embodiments, the transgene encoding TREM2 includes a codon-optimized TREM2 polynucleotide having at least 99% sequence identity to the nucleic acid sequence of SEQ ID NO. 12. In some embodiments, the transgene encoding TREM2 includes a codon-optimized TREM2 polynucleotide of SEQ ID NO. 12.

In some embodiments, the transgene encodes two or more TREM2 transgenes (e.g., at least 2, 3, 4, 5, 6, 7, 8, 9, 10 or more TREM2 transgenes). In some embodiments, the transgene encodes from two to ten TREM2 transgenes (e.g., 2, 3, 4, 5, 6, 7, 8, 9, or 10 TREM2 transgenes). In some embodiments, the transgene encodes from two to five TREM2 transgenes (e.g., 2, 3, 4, or 5 TREM2 transgenes). In some embodiments, the transgene encodes two TREM2 transgenes. In some embodiments, the TREM2 transgenes are expressed from a single, polycistronic expression cassette. In some embodiments, the TREM2 transgenes are separated from one another by way of one or more (e.g., 1, 2, 3, 4, 5, 6, 7, 8, 9, or more) IRES. In some embodiments, the TREM2 transgenes are expressed from one or more (e.g., 1, 2, 3, 4, 5, 6, 7, 8, 9, 10, or more) monocistronic expression cassettes.

In some embodiments, the polynucleotide encoding TREM2 encodes sTREM2. In some embodiments, the polynucleotide encoding TREM2 encodes the TREM2-CTF. In some embodiments, the polynucleotide encoding TREM2 encodes the TREM2-ICD. In some embodiments, the polynucleotide encoding TREM2 encodes the TREM2-T2β peptide. In some embodiment, the polynucleotide encoding TREM2 encodes a TREM2 polypeptide lacking a functional ectodomain cleavage site. In some embodiment, the polynucleotide encoding TREM2 encodes a TREM2 polypeptide lacking a functional intramembrane cleavage site within the TREM2-CTF. In some embodiments, the polynucleotide encoding wild type TREM2 may be a codon-optimized polynucleotide to confer resistance against degradation by nucleases and inhibitory RNAs directed to endogenous TREM2, as described in detail below.

Wild type human TREM2 may have the canonical amino acid sequence of (UniProt identifier number: Q9NZC2-1):

(SEQ ID NO. 1)
MEPLRLLILLFVTELSGAHNTTVFQGVAGQSLQVSCPYDSMKHWGRRKAW
CRQLGEKGPCQRVVSTHNLWLLSFLRRWNGSTAITDDTLGGTLTITLRNL
QPHDAGLYQCQSLHGSEADTLRKVLVEVLADPLDHRDAGDLWFPGESESF
EDAHVEHSISRSLLEGEIPFPPTSILLLLACIFLIKILAASALWAAAWHG
QKPGTHPPSELDCGHDPGYQLQTLPGLRDT

Additionally or alternatively, human TREM2 may also have the amino acid sequence of (UniProt identifier number: Q9NZC2-2):

(SEQ ID NO. 2)
MEPLRLLILLFVTELSGAHNTTVFQGVAGQSLQVSCPYDSMKHWGRRKAW
CRQLGEKGPCQRVVSTHNLWLLSFLRRWNGSTAITDDTLGGTLTITLRNL
QPHDAGLYQCQSLHGSEADTLRKVLVEVLADPLDHRDAGDLWFPGESESF
EDAHVEHSISRAERHVKEDDGRKSPGEVPPGTSPACILATWPPGLLVLLW
QETTLPEHCFSWTLEAGTG

Additionally or alternatively, human TREM2 may also have the amino acid sequence of (UniProt identifier number: Q9NZC2-3):

(SEQ ID NO. 3)
MEPLRLLILLFVTELSGAHNTTVFQGVAGQSLQVSCPYDSMKHWGRRKAW
CRQLGEKGPCQRVVSTHNLWLLSFLRRWNGSTAITDDTLGGTLTITLRNL
QPHDAGLYQCQSLHGSEADTLRKVLVEVLADPLDHRDAGDLWFPGESESF
EDAHVEHSISRPSQGSHLPSCLSKEPLGRRNPLPTHFHPSPPGLHLSHQD
SSSQRPLGCSLAWTEARDTSTQ

The polynucleotide encoding TREM2 may have the nucleic acid sequence of (Ensembl identifier number: ENST00000373113.7):

(SEQ ID NO. 4)
TGACATGCCTGATCCTCTCTTTTCTGCAGTTCAAGGGAAAGACGAGATC
TTGCACAAGGCACTCTGCTTCTGCCCTTGGCTGGGGAAGGGTGGCATGG
AGCCTCTCCGGCTGCTCATCTTACTCTTTGTCACAGAGCTGTCCGGAGC
CCACAACACCACAGTGTTCCAGGGCGTGGCGGGCCAGTCCCTGCAGGTG
TCTTGCCCCTATGACTCCATGAAGCACTGGGGGAGGCGCAAGGCCTGGT
GCCGCCAGCTGGGAGAGAAGGGCCCATGCCAGCGTGTGGTCAGCACGCA
CAACTTGTGGCTGCTGTCCTTCCTGAGGAGGTGGAATGGGAGCACAGCC
ATCACAGACGATACCCTGGGTGGCACTCTCACCATTACGCTGCGGAATC
TACAACCCCATGATGCGGGTCTCTACCAGTGCCAGAGCCTCCATGGCAG
TGAGGCTGACACCCTCAGGAAGGTCCTGGTGGAGGTGCTGGCAGACCCC
CTGGATCACCGGGATGCTGGAGATCTCTGGTTCCCCGGGGAGTCTGAGA
GCTTCGAGGATGCCCATGTGGAGCACAGCATCTCCAGGAGCCTCTTGGA
AGGAGAAATCCCCTTCCCACCCACTTCCATCCTTCTCCTCCTGGCCTGC
ATCTTTCTCATCAAGATTCTAGCAGCCAGCGCCCTCTGGGCTGCAGCCT
GGCATGGACAGAAGCCAGGGACACATCCACCCAGTGAACTGGACTGTGG
CCATGACCCAGGGTATCAGCTCCAAACTCTGCCAGGGCTGAGAGACACG
TGAAGGAAGATGATGGGAGGAAAAGCCCAGGAGAAGTCCCACCAGGGAC
CAGCCCAGCCTGCATACTTGCCACTTGGCCACCAGGACTCCTTGTTCTG
CTCTGGCAAGAGACTACTCTGCCTGAACACTGCTTCTCCTGGACCCTGG
AAGCAGGGACTGGTTGAGGGAGTGGGGAGGTGGTAAGAACACCTGACAA
CTTCTGAATATTGGACATTTTAAACACTTACAAATAAATCCAAGACTGT
CATATTTAGCTGGAT

Additionally or alternatively, human TREM2 may have the nucleotide sequence of (Ensembl identifier number: ENST00000338469.3)

(SEQ ID NO. 5)
GGGCAGCGCCTGACATGCCTGATCCTCTCTTTTCTGCAGTTCAAGGGAA
AGACGAGATCTTGCACAAGGCACTCTGCTTCTGCCCTTGGCTGGGGAAG
GGTGGCATGGAGCCTCTCCGGCTGCTCATCTTACTCTTTGTCACAGAGC
TGTCCGGAGCCCACAACACCACAGTGTTCCAGGGCGTGGCGGGCCAGTC
CCTGCAGGTGTCTTGCCCCTATGACTCCATGAAGCACTGGGGGAGGCGC
AAGGCCTGGTGCCGCCAGCTGGGAGAGAAGGGCCCATGCCAGCGTGTGG
TCAGCACGCACAACTTGTGGCTGCTGTCCTTCCTGAGGAGGTGGAATGG
GAGCACAGCCATCACAGACGATACCCTGGGTGGCACTCTCACCATTACG
CTGCGGAATCTACAACCCCATGATGCGGGTCTCTACCAGTGCCAGAGCC
TCCATGGCAGTGAGGCTGACACCCTCAGGAAGGTCCTGGTGGAGGTGCT
GGCAGACCCCCTGGATCACCGGGATGCTGGAGATCTCTGGTTCCCCGGG
GAGTCTGAGAGCTTCGAGGATGCCCATGTGGAGCACAGCATCTCCAGGG
CTGAGAGACACGTGAAGGAAGATGATGGGAGGAAAAGCCCAGGAGAAGT
CCCACCAGGGACCAGCCCAGCCTGCATACTTGCCACTTGGCCACCAGGA
CTCCTTGTTCTGCTCTGGCAAGAGACTACTCTGCCTGAACACTGCTTCT
CCTGGACCCTGGAAGCAGGGACTGGTTGAGGGAGTGGGGAGGTGGTAAG
AACACCTGACAACTTCTGAATATTGGACATTTTAAACACTTACAAATAA
ATCCAAGACTGTCATATTTAGCTGGAT

Additionally or alternatively, human TREM2 may have the nucleotide sequence of (Ensembl identifier number: ENST00000373122.8):

(SEQ ID NO. 6)
CCTTGGCTGGGGAAGGGTGGCATGGAGCCTCTCCGGCTGCTCATCTTAC
TCTTTGTCACAGAGCTGTCCGGAGCCCACAACACCACAGTGTTCCAGGG
CGTGGCGGGCCAGTCCCTGCAGGTGTCTTGCCCCTATGACTCCATGAAG
CACTGGGGGAGGCGCAAGGCCTGGTGCCGCCAGCTGGGAGAGAAGGGCC
CATGCCAGCGTGTGGTCAGCACGCACAACTTGTGGCTGCTGTCCTTCCT
GAGGAGGTGGAATGGGAGCACAGCCATCACAGACGATACCCTGGGTGGC
ACTCTCACCATTACGCTGCGGAATCTACAACCCCATGATGCGGGTCTCT
ACCAGTGCCAGAGCCTCCATGGCAGTGAGGCTGACACCCTCAGGAAGGT
CCTGGTGGAGGTGCTGGCAGACCCCCTGGATCACCGGGATGCTGGAGAT
CTCTGGTTCCCCGGGGAGTCTGAGAGCTTCGAGGATGCCCATGTGGAGC
ACAGCATCTCCAGGCCATCTCAAGGCTCCCATCTGCCTTCTTGTCTCTC
CAAGGAGCCTCTTGGAAGGAGAAATCCCCTTCCCACCCACTTCCATCCT
TCTCCTCCTGGCCTGCATCTTTCTCATCAAGATTCTAGCAGCCAGCGCC
CTCTGGGCTGCAGCCTGGCATGGACAGAAGCCAGGGACACATCCACCCA
GTGAACTGGACTGTGGCCATGACCCAGGGTATCAGCTCCAAACTCTGCC
AGGGCTGAGAGACACGTGAAGGAAGATGATGGGAGGAAAAGCCCAGGAG
AAGTCCCACCAGGGACCAGCCCAGCCTGCATACTTGCCACTTGGCCACC
AGGACTCCTTGTTCTGCTCTGGCAAGAGACTACTCTGCCTGAACACTGC
TTCTCCTGGACCCTGGAAGCAGGGACTGGTTGAGGGAGTGGGGAGGTGG
TAAGAACACCTGACAACTTCTGAATATTGGACATTTTAAACACTTACAA
ATAAATCCAAGACTGTCATATTTAGCTGGATA

Additionally or alternatively, the polynucleotide encoding TREM2 may have the sequence of:

(SEQ ID NO. 7)
ATGGAGCCTCTCCGGCTGCTCATCTTACTCTTTGTCACAGAGCTGTCCG
GAGCCCACAACACCACAGTGTTCCAGGGCGTGGCGGGCCAGTCCCTGCA
GGTGTCTTGCCCCTATGACTCCATGAAGCACTGGGGGAGGCGCAAGGCC
TGGTGCCGCCAGCTGGGAGAGAAGGGCCCATGCCAGCGTGTGGTCAGCA
CGCACAACTTGTGGCTGCTGTCCTTCCTGAGGAGGTGGAATGGGAGCAC
AGCCATCACAGACGATACCCTGGGTGGCACTCTCACCATTACGCTGCGG
AATCTACAACCCCATGATGCGGGTCTCTACCAGTGCCAGAGCCTCCATG
GCAGTGAGGCTGACACCCTCAGGAAGGTCCTGGTGGAGGTGCTGGCAGA
CCCCCTGGATCACCGGGATGCTGGAGATCTCTGGTTCCCCGGGGAGTCT
GAGAGCTTCGAGGATGCCCATGTGGAGCACAGCATCTCCAGGAGCCTCT
TGGAAGGAGAAATCCCCTTCCCACCCACTTCCATCCTTCTCCTCCTGGC
CTGCATCTTTCTCATCAAGATTCTAGCAGCCAGCGCCCTCTGGGCTGCA
GCCTGGCATGGACAGAAGCCAGGGACACATCCACCCAGTGAACTGGACT
GTGGCCATGACCCAGGGTATCAGCTCCAAACTCTGCCAGGGCTGAGAGA
CACGTGATGA

Additionally or alternatively, the polynucleotide encoding TREM2 may have the codon-optimized nucleotide sequence of SEQ ID NO. 8:

(SEQ ID NO. 8)
ATGGAGCCTCTGAGACTGCTGATTCTGCTGTTTGTCACTGAACTGAGCG
GCGCACATAATACCACTGTCTTCCAGGGCGTCGCTGGGCAGTCTCTGCA
GGTGAGCTGCCCCTACGACTCTATGAAGCACTGGGGCCGGAGAAAGGCA
TGGTGCCGGCAGCTGGGAGAGAAGGGACCTTGTCAGAGAGTGGTGAGCA
CCCACAACCTGTGGCTGCTGTCCTTCCTGAGGCGCTGGAATGGCTCTAC
AGCCATCACCGACGATACACTGGGCGGCACCCTGACAATCACCCTGAGG
AACCTGCAGCCTCACGACGCAGGCCTGTATCAGTGCCAGTCCCTGCACG
GCTCTGAGGCCGATACACTGAGGAAGGTGCTGGTGGAGGTGCTGGCCGA
CCCTCTGGATCACAGGGACGCAGGCGATCTGTGGTTCCCAGGCGAGAGC
GAGTCCTTTGAGGATGCCCACGTGGAGCACTCTATCAGCCGGTCCCTGC
TGGAGGGAGAGATCCCATTCCCCCCTACCAGCATCCTGCTGCTGCTGGC
CTGTATCTTTCTGATCAAGATCCTGGCAGCATCCGCCCTGTGGGCAGCA
GCCTGGCACGGACAGAAGCCAGGAACACACCCACCATCCGAGCTGGATT
GCGGACATGACCCCGGCTACCAGCTGCAGACACTGCCTGGCCTGAGGGA
TACATGATGA

Additionally or alternatively, the polynucleotide encoding TREM2 may have the nucleotide sequence of:

(SEQ ID NO. 9)
ATGGAGCCTCTCCGGCTGCTCATCTTACTCTTTGTCACAGAGCTGTCCG
GAGCCCACAACACCACAGTGTTCCAGGGCGTGGCGGGCCAGTCCCTGCA
GGTGTCTTGCCCCTATGACTCCATGAAGCACTGGGGGAGGCGCAAGGCC
TGGTGCCGCCAGCTGGGAGAGAAGGGCCCATGCCAGCGTGTGGTCAGCA
CGCACAACTTGTGGCTGCTGTCCTTCCTGAGGAGGTGGAATGGGAGCAC
AGCCATCACAGACGATACCCTGGGTGGCACTCTCACCATTACGCTGCGG
AATCTACAACCCCATGATGCGGGTCTCTACCAGTGCCAGAGCCTCCATG
GCAGTGAGGCTGACACCCTCAGGAAGGTCCTGGTGGAGGTGCTGGCAGA
CCCCCTGGATCACCGGGATGCTGGAGATCTCTGGTTCCCCGGGGAGTCT
GAGAGCTTCGAGGATGCCCATGTGGAGCACAGCATCTCCAGGGCTGAGA
GACACGTGAAGGAAGATGATGGGAGGAAAAGCCCAGGAGAAGTCCCACC
AGGGACCAGCCCAGCCTGCATACTTGCCACTTGGCCACCAGGACTCCTT
GTTCTGCTCTGGCAAGAGACTACTCTGCCTGAACACTGCTTCTCCTGGA
CCCTGGAAGCAGGGACTGGTTGATGA

Additionally or alternatively, the polynucleotide encoding TREM2 may have the codon-optimized nucleotide sequence of SEQ ID NO. 10:

(SEQ ID NO. 10)
ATGGAGCCTCTGCGGCTGCTGATCCTGCTGTTCGTGACCGAGCTGTCCG
GCGCCCACAACACCACAGTGTTTCAGGGAGTGGCAGGACAGTCCCTGCA
GGTGTCTTGCCCATACGACTCTATGAAGCACTGGGGCCGGAGAAAGGCA
TGGTGCAGGCAGCTGGGAGAGAAGGGACCATGTCAGCGCGTGGTGTCTA
CACACAACCTGTGGCTGCTGAGCTTCCTGAGGCGCTGGAATGGCTCCAC
AGCCATCACCGACGATACACTGGGCGGCACCCTGACAATCACCCTGAGG
AATCTGCAGCCACACGACGCCGGCCTGTATCAGTGTCAGAGCCTGCACG
GCTCCGAGGCAGATACCCTGCGGAAGGTGCTGGTGGAGGTGCTGGCCGA
CCCCCTGGATCACAGAGACGCAGGCGATCTGTGGTTCCCTGGCGAGAGC
GAGTCCTTTGAGGATGCCCACGTGGAGCACTCTATCAGCCGGGCCGAGA
GACACGTGAAGGAGGACGATGGAAGGAAGTCTCCTGGAGAGGTGCCACC
TGGAACCAGCCCAGCATGCATCCTGGCAACATGGCCACCAGGCCTGCTG
GTGCTGCTGTGGCAGGAGACAACACTGCCCGAGCACTGTTTTTCCTGGA
CCCTGGAGGCCGGCACAGGCTGATGA

Additionally or alternatively, the polynucleotide encoding TREM2 may have the nucleotide sequences of SEQ ID NO. 7 and SEQ ID NO. 9 separated by an IRES sequence, collectively having the nucleotide sequence of:

(SEQ ID NO. 11)
ATGGAGCCTCTCCGGCTGCTCATCTTACTCTTTGTCACAGAGCTGTCCG
GAGCCCACAACACCACAGTGTTCCAGGGCGTGGCGGGCCAGTCCCTGCA
GGTGTCTTGCCCCTATGACTCCATGAAGCACTGGGGGAGGCGCAAGGCC
TGGTGCCGCCAGCTGGGAGAGAAGGGCCCATGCCAGCGTGTGGTCAGCA
CGCACAACTTGTGGCTGCTGTCCTTCCTGAGGAGGTGGAATGGGAGCAC
AGCCATCACAGACGATACCCTGGGTGGCACTCTCACCATTACGCTGCGG
AATCTACAACCCCATGATGCGGGTCTCTACCAGTGCCAGAGCCTCCATG
GCAGTGAGGCTGACACCCTCAGGAAGGTCCTGGTGGAGGTGCTGGCAGA
CCCCCTGGATCACCGGGATGCTGGAGATCTCTGGTTCCCCGGGGAGTCT
GAGAGCTTCGAGGATGCCCATGTGGAGCACAGCATCTCCAGGAGCCTCT
TGGAAGGAGAAATCCCCTTCCCACCCACTTCCATCCTTCTCCTCCTGGC
CTGCATCTTTCTCATCAAGATTCTAGCAGCCAGCGCCCTCTGGGCTGCA
GCCTGGCATGGACAGAAGCCAGGGACACATCCACCCAGTGAACTGGACT
GTGGCCATGACCCAGGGTATCAGCTCCAAACTCTGCCAGGGCTGAGAGA
CACGTGATGATTCCGCCCCTCTCCCTCCCCCCCCCCTAACGTTACTGGC
CGAAGCCGCTTGGAATAAGGCCGGTGTGCGTTTGTCTATATGTTATTTT
CCACCATATTGCCGTCTTTTGGCAATGTGAGGGCCCGGAAACCTGGCCC
TGTCTTCTTGACGAGCATTCCTAGGGGTCTTTCCCCTCTCGCCAAAGGA
ATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCC
CCCACCTGGCGACAGGTGCCTCTGCGGCCAAAAGCCACGTGTATAAGAT
ACACCTGCAAAGGCGGCACAACCCCAGTGCCACGTTGTGAGTTGGATAG
TTGTGGAAAGAGTCAAATGGCTCTCCTCAAGCGTATTCAACAAGGGGCT
GAAGGATGCCCAGAAGGTACCCCATTGTATGGGATCTGATCTGGGGCCT
CGGTGCACATGCTTTACATGTGTTTAGTCGAGGTTAAAAAACGTCTAGG
CCCCCCGAACCACGGGGACGTGGTTTTCCTTTGAAAAACACGATGATAA
TAGCCACCATGGAGCCTCTCCGGCTGCTCATCTTACTCTTTGTCACAGA
GCTGTCCGGAGCCCACAACACCACAGTGTTCCAGGGCGTGGCGGGCCAG
TCCCTGCAGGTGTCTTGCCCCTATGACTCCATGAAGCACTGGGGGAGGC
GCAAGGCCTGGTGCCGCCAGCTGGGAGAGAAGGGCCCATGCCAGCGTGT
GGTCAGCACGCACAACTTGTGGCTGCTGTCCTTCCTGAGGAGGTGGAAT
GGGAGCACAGCCATCACAGACGATACCCTGGGTGGCACTCTCACCATTA
CGCTGCGGAATCTACAACCCCATGATGCGGGTCTCTACCAGTGCCAGAG
CCTCCATGGCAGTGAGGCTGACACCCTCAGGAAGGTCCTGGTGGAGGTG
CTGGCAGACCCCCTGGATCACCGGGATCTGGAGATCTCTGGTTCCCCGG
GGGAGTCTGAGAGCTTCGAGGATGCCCATGTGGAGCACAGCATCTCCAG
GGCTGAGAGACACGTGAAGGAAGATGATGGGAGGAAAAGCCCAGGAGAA
GTCCCACCAGGGACCAGCCCAGCCTGCATACTTGCCACTTGGCCACCAG
GACTCCTTGTTCTGCTCTGGCAAGAGACTACTCTGCCTGAACACTGCTT
CTCCTGGACCCTGGAAGCAGGGACTGGTTGATGA

Additionally or alternatively, the polynucleotide encoding TREM2 may have the nucleotide sequences of SEQ ID NO. 8 and SEQ ID NO. 10 separated by an IRES sequence, collectively having the nucleotide sequence of:

(SEQ ID NO. 12)
ATGGAGCCTCTGAGACTGCTGATTCTGCTGTTTGTCACTGAACTGAGCG
GCGCACATAATACCACTGTCTTCCAGGGCGTCGCTGGGCAGTCTCTGCA
GGTGAGCTGCCCCTACGACTCTATGAAGCACTGGGGCCGGAGAAAGGCA
TGGTGCCGGCAGCTGGGAGAGAAGGGACCTTGTCAGAGAGTGGTGAGCA
CCCACAACCTGTGGCTGCTGTCCTTCCTGAGGCGCTGGAATGGCTCTAC
AGCCATCACCGACGATACACTGGGCGGCACCCTGACAATCACCCTGAGG
AACCTGCAGCCTCACGACGCAGGCCTGTATCAGTGCCAGTCCCTGCACG
GCTCTGAGGCCGATACACTGAGGAAGGTGCTGGTGGAGGTGCTGGCCGA
CCCTCTGGATCACAGGGACGCAGGCGATCTGTGGTTCCCAGGCGAGAGC
GAGTCCTTTGAGGATGCCCACGTGGAGCACTCTATCAGCCGGTCCCTGC
TGGAGGGAGAGATCCCATTCCCCCCTACCAGCATCCTGCTGCTGCTGGC
CTGTATCTTTCTGATCAAGATCCTGGCAGCATCCGCCCTGTGGGCAGCA
GCCTGGCACGGACAGAAGCCAGGAACACACCCACCATCCGAGCTGGATT
GCGGACATGACCCCGGCTACCAGCTGCAGACACTGCCTGGCCTGAGGGA
TACATGATGATTCCGCCCCTCTCCCTCCCCCCCCCCTAACGTTACTGGC
CGAAGCCGCTTGGAATAAGGCCGGTGTGCGTTTGTCTATATGTTATTTT
CCACCATATTGCCGTCTTTTGGCAATGTGAGGGCCCGGAAACCTGGCCC
TGTCTTCTTGACGAGCATTCCTAGGGGTCTTTCCCCTCTCGCCAAAGGA
ATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCC
CCCACCTGGCGACAGGTGCCTCTGCGGCCAAAAGCCACGTGTATAAGAT
ACACCTGCAAAGGCGGCACAACCCCAGTGCCACGTTGTGAGTTGGATAG
TTGTGGAAAGAGTCAAATGGCTCTCCTCAAGCGTATTCAACAAGGGGCT
GAAGGATGCCCAGAAGGTACCCCATTGTATGGGATCTGATCTGGGGCCT
CGGTGCACATGCTTTACATGTGTTTAGTCGAGGTTAAAAAACGTCTAGG
CCCCCCGAACCACGGGGACGTGGTTTTCCTTTGAAAAACACGATGATAA
TAGCCACCATGGAGCCTCTGCGGCTGCTGATCCTGCTGTTCGTGACCGA
GCTGTCCGGCGCCCACAACACCACAGTGTTTCAGGGAGTGGCAGGACAG
TCCCTGCAGGTGTCTTGCCCATACGACTCTATGAAGCACTGGGGCCGGA
GAAAGGCATGGTGCAGGCAGCTGGGAGAGAAGGGACCATGTCAGCGCGT
GGTGTCTACACACAACCTGTGGCTGCTGAGCTTCCTGAGGCGCTGGAAT
GGCTCCACAGCCATCACCGACGATACACTGGGCGGCACCCTGACAATCA
CCCTGAGGAATCTGCAGCCACACGACGCCGGCCTGTATCAGTGTCAGAG
CCTGCACGGCTCCGAGGCAGATACCCTGCGGAAGGTGCTGGTGGAGGTG
CTGGCCGACCCCCTGGATCACAGAGACGCAGGCGATCTGTGGTTCCCTG
GCGAGAGCGAGTCCTTTGAGGATGCCCACGTGGAGCACTCTATCAGCCG
GGCCGAGAGACACGTGAAGGAGGACGATGGAAGGAAGTCTCCTGGAGAG
GTGCCACCTGGAACCAGCCCAGCATGCATCCTGGCAACATGGCCACCAG
GCCTGCTGGTGCTGCTGTGGCAGGAGACAACACTGCCCGAGCACTGTTT
TTCCTGGACCCTGGAGGCCGGCACAGGCTGATGA

According to the methods described herein, a subject can be administered a cell containing a transgene that includes a polynucleotide encoding a polypeptide having any one of amino acid sequences of SEQ ID NOS. 1-3, or a polynucleotide encoding a polypeptide having at least 85% sequence identity (e.g., 85%, 90%, 95%, 96%, 97%, 98%, 99%, or more, sequence identity) to any one of the amino acid sequences of SEQ ID NOS. 1-3, or a polynucleotide encoding a polypeptide that contains one or more conservative amino acid substitutions (e.g., 1, 2, 3, 4, 5, 6, 7, 8, 9, or 10 or more conservative amino acid substitutions) relative to any one of SEQ ID NOS. 1-3, provided that the TREM2 analog encoded retains the therapeutic function of wild type TREM2. The activity of TREM2 is important for normal microglial phagocytic competency and regulation of inflammatory cytokine production. Loss of TREM2 leads to altered neuro-immune responses and neurodegeneration.

### High Expression Constitutive Promoter

In some embodiments of the disclosure, the TREM2 transgene may be expressed at sufficiently high levels so as to elicit a therapeutic benefit. Accordingly, transgene expression may be mediated by a promoter sequence capable of driving robust expression of the disclosed TREM2 constructs in target cells (e.g., pluripotent or multipotent cells). The present disclosure features a heterologous promoter sequence suitable for use with the methods and compositions disclosed herein. The term “heterologous promoter”, as used herein, refers to a promoter that is not found to be operatively linked to a given encoding sequence in nature. Useful heterologous control sequences generally include those derived from sequences encoding mammalian or viral genes.

For purposes of the present disclosure, heterologous promoters, such as promoters capable of driving TREM2 expression specifically in a population of cells (e.g., pluripotent cells, ESCs, iPSCs, multipotent cells, CD34+ cells, HSCs, myeloid cells (e.g., MPCs), BLPCs, monocytes, macrophages, microglial progenitor cells, or microglia) and the like will be of particular use. Accordingly, a promoter sequence may be derived in its entirety from a native gene or may be composed of different elements derived from different naturally-occurring promoters. Alternatively, the promoter may include a synthetic polynucleotide.

Thus, the present disclosure features an MND promoter that can be incorporated into an expression cassette encoding a TREM2 transgene of the disclosure to drive robust transgene expression specifically in target cells. The MND promoter is a synthetic promoter sequence derived from a myeloproliferative sarcoma virus (MSV) and contains an MSV enhancer, a U3 region of a Maloney Murine Leukemia Virus, a deletion of a negative control region, and a substitution of a dl587rev primer binding site. The MND promoter may have a sequence of SEQ ID NO. 14 or may be a variant thereof having at least 85% (e.g., at least 86%, 90%, 95%, 96%, 97%, 98%, 99%, or more) sequence identity to the nucleic acid sequence of SEQ ID NO. 14, as is shown below.

(SEQ ID NO. 14)
GATCAAGGTTAGGAACAGAGAGACAGGAGAATATGGGCCAAACAGGATA
TCTGTGGTAAGCAGTTCCTGCCCCGGCTCAGGGCCAAGAACAGTTGGAA
CAGCAGAATATGGGCCAAACAGGATATCTGTGGTAAGCAGTTCCTGCCC
CGGCTCAGGGCCAAGAACAGATGGTCCCCAGATGCGGTCCCGCCCTCAG
CAGTTTCTAGAGAACCATCAGATGTTTCCAGGGTGCCCCAAGGACCTGA
AATGACCCTGTGCCTTATTTGAACTAACCAATCAGTTCGCTTCTCGCTT
CTGTTCGCGCGCTTCTGCTCCCCGAGCTCAATAAAAGAGCCCACAACCC
CTCACTCGGCGCGCCAGTCCTCCGATAGACTGCGTCGCCCGG

Alternatively, the MND promoter may have a sequence of SEQ ID NO. 16 or may be a variant thereof having at least 85% (e.g., at least 86%, 90%, 95%, 96%, 97%, 98%, 99%, or more) sequence identity to the nucleic acid sequence of SEQ ID NO. 14, as is shown below.

(SEQ ID NO. 16)
TTATTTAGTCTCCAGAAAAAGGGGGGAATGAAAGACCCCACCTGTAGGT
TTGGCAAGCTAGGATCAAGGTTAGGAACAGAGAGACAGCAGAATATGGG
CCAAACAGGATATCTGTGGTAAGCAGTTCCTGCCCCGGCTCAGGGCCAA
GAACAGTTGGAACAGCAGAATATGGGCCAAACAGGATATCTGTGGTAAG
CAGTTCCTGCCCCGGCTCAGGGCCAAGAACAGATGGTCCCCAGATGCGG
TCCCGCCCTCAGCAGTTTCTAGAGAACCATCAGATGTTTCCAGGGTGCC
CCAAGGACCTGAAATGACCCTGTGCCTTATTTGAACTAACCAATCAGTT
CGCTTCTCGCTTCTGTTCGCGCGCTTCTGCTCCCCGAGCTCAATAAAAG
AGCCCA

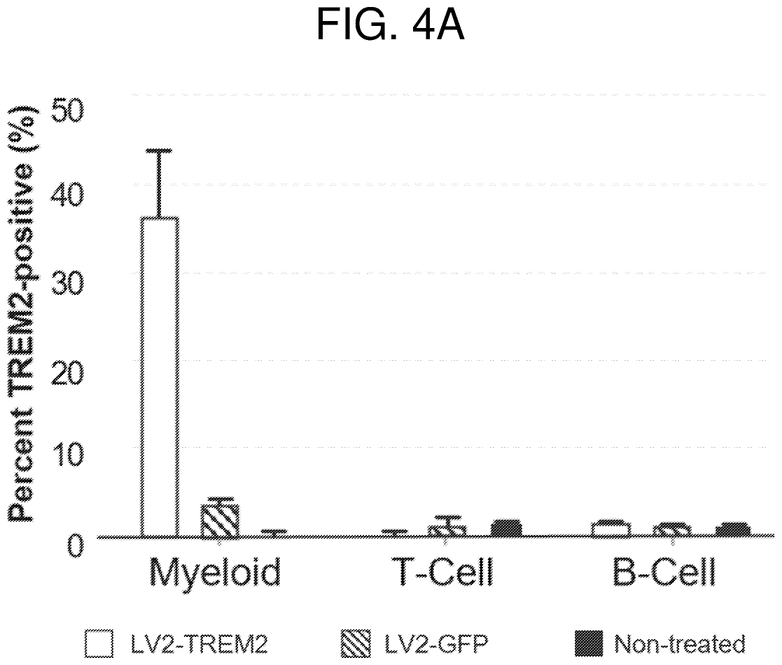

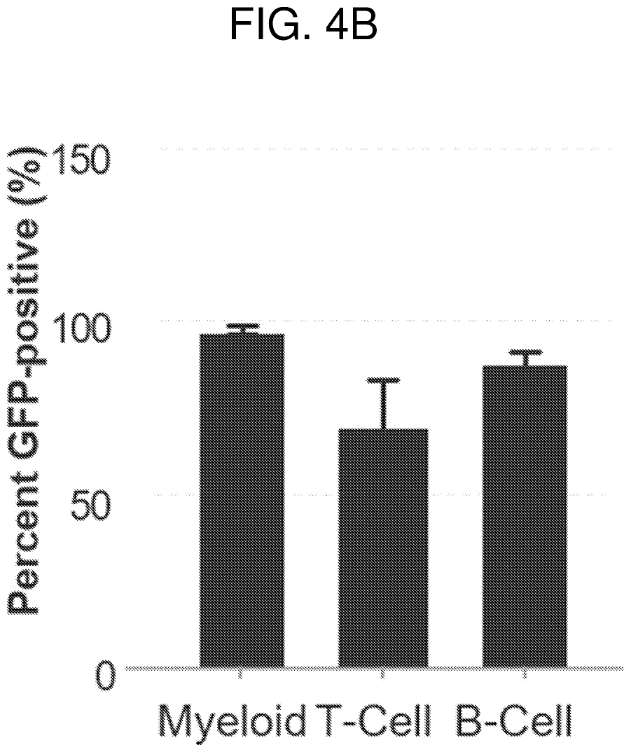

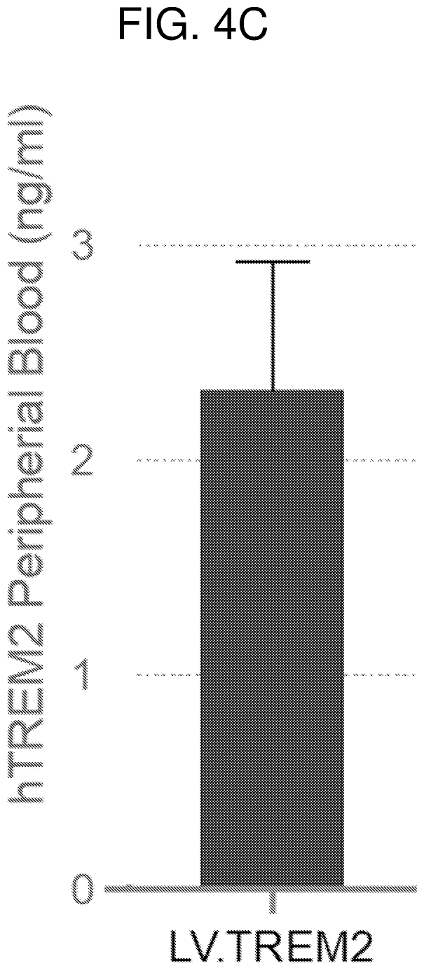

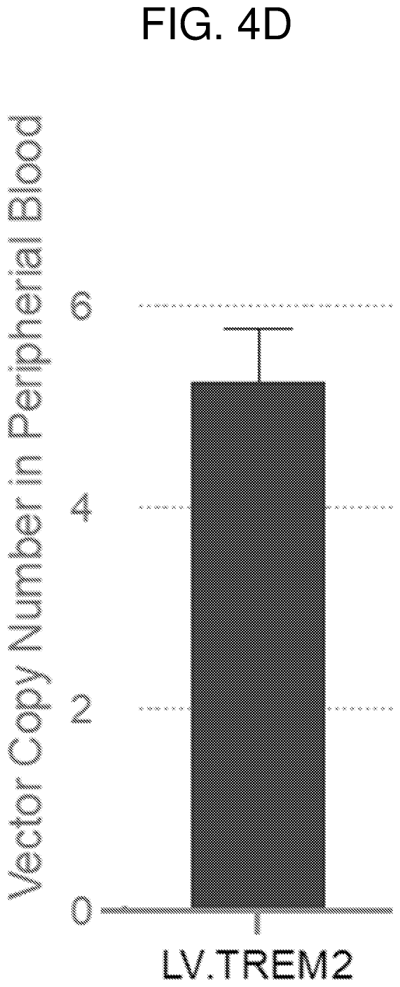

The present inventors have demonstrated that an expression vector containing a TREM2 transgene operably linked to an MND promoter was able to selectively drive TREM2 transgene expression selectively in cells of the myeloid lineage in vivo at levels that were substantially higher than the near-zero expression levels observed in cells of the lymphoid lineage (i.e., T-cells and B-cells; see Example 1 and FIG. 4). Accordingly, the present disclosure contemplates expression constructs encoding a TREM2 protein (e.g., a TREM2 protein having any one of the amino acid sequences of SEQ ID NOs. 1-3 or a variant thereof having at least 85% (e.g., at least 86%, 90%, 95%, 96%, 97%, 98%, 99%, or more) sequence identity to the nucleic acid sequence of SEQ ID NOs. 1-3 operably linked to an MND promoter (e.g., having the nucleic acid sequence of SEQ ID NO. 14 or SEQ ID NO. 16 a variant thereof having at least 85% (e.g., at least 86%, 90%, 95%, 96%, 97%, 98%, 99%, or more) sequence identity to the nucleic acid sequence of SEQ ID NO. 14 or SEQ ID NO. 16. In some embodiments, the MND promoter has at least 90% (e.g., at least 91%, 95%, 96%, 97%, 98%, 99%, or more) sequence identity to the nucleic acid sequence of SEQ ID NO. 14. In some embodiments, the MND promoter has at least 95% (e.g., at least 96%, 97%, 98%, 99%, or more) sequence identity to the nucleic acid sequence of SEQ ID NO. 14. In some embodiments, the MND promoter has at least 99% sequence identity to the nucleic acid sequence of SEQ ID NO. 14. In some embodiments, the MND promoter has the nucleic acid sequence of SEQ ID NO. 14. In some embodiments, the transgene encoding TREM2 includes a codon-optimized TREM2 polynucleotide having at least 98% (e.g., at least 99% or more) sequence identity to the nucleic acid sequence of SEQ ID NO. 12.

In some embodiments, the MND promoter has at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO. 16. In some embodiments, the MND promoter has at least 90% (e.g., at least 91%, 95%, 96%, 97%, 98%, 99%, or more) sequence identity to the nucleic acid sequence of SEQ ID NO. 16. In some embodiments, the MND promoter has at least 95% (e.g., at least 96%, 97%, 98%, 99%, or more) sequence identity to the nucleic acid sequence of SEQ ID NO. 16. In some embodiments, the MND promoter has at least 99% sequence identity to the nucleic acid sequence of SEQ ID NO. 16. In some embodiments, the MND promoter has the nucleic acid sequence of SEQ ID NO. 16.

An exemplary expression vector construct containing a codon-optimized TREM2-encoding polynucleotide (SEQ ID NO. 8) operably linked to an MND promoter (SEQ ID NO. 14) is provided in SEQ ID NO. 15, as is shown below. The codon-optimized TREM2-encoding polynucleotide of SEQ ID NO. 8 encodes a wild-type TREM2 peptide of SEQ ID NO. 1.

(SEQ ID NO. 15)
CGCGTTGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAA
CCCTATCTCGGTCTATTCTTTTGATTTATAAGGGATTTTGCCGATTTCGGCCTATTGG
TTAAAAAATGAGCTGATTTAACAAAAATTTAACGCGAATTTTAACAAAATATTAACGTT
TACAATTTCCCAGGTGGCACTTTTCGGGGAAATGTGCGCGGAACCCCTATTTGTTTA
TTTTTCTAAATACATTCAAATATGTATCCGCTCATGAGACAATAACCCTGATAAATGCT
TCAATAATATTGAAAAAGGAAGAGTATGATTGAACAAGATGGATTGCACGCAGGTTC
TCCGGCCGCTTGGGTGGAGAGGCTATTCGGCTATGACTGGGCACAACAGACAATC
GGCTGCTCTGATGCCGCCGTGTTCCGGCTGTCAGCGCAGGGGCGCCCGGTTCTTT
TTGTCAAGACCGACCTGTCCGGTGCCCTGAATGAACTGCAAGACGAGGCAGCGCG
GCTATCGTGGCTGGCCACGACGGGCGTTCCTTGCGCAGCTGTGCTCGACGTTGTC
ACTGAAGCGGGAAGGGACTGGCTGCTATTGGGCGAAGTGCCGGGGCAGGATCTCC
TGTCATCTCACCTTGCTCCTGCCGAGAAAGTATCCATCATGGCTGATGCAATGCGGC
GGCTGCATACGCTTGATCCGGCTACCTGCCCATTCGACCACCAAGCGAAACATCGC
ATCGAGCGAGCACGTACTCGGATGGAAGCCGGTCTTGTCGATCAGGATGATCTGGA
CGAAGAGCATCAGGGGCTCGCGCCAGCCGAACTGTTCGCCAGGCTCAAGGCGAGC
ATGCCCGACGGCGAGGATCTCGTCGTGACCCATGGCGATGCCTGCTTGCCGAATAT
CATGGTGGAAAATGGCCGCTTTTCTGGATTCATCGACTGTGGCCGGCTGGGTGTGG
CGGACCGCTATCAGGACATAGCGTTGGCTACCCGTGATATTGCTGAAGAGCTTGGC
GGCGAATGGGCTGACCGCTTCCTCGTGCTTTACGGTATCGCCGCTCCCGATTCGCA
GCGCATCGCCTTCTATCGCCTTCTTGACGAGTTCTTCTGACTGTCAGACCAAGTTTA
CTCATATATACTTTAGATTGATTTAAAACTTCATTTTTAATTTAAAAGGATCTAGGTGA
AGATCCTTTTTGATAATCTCATGACCAAAATCCCTTAACGTGAGTTTTCGTTCCACTG
AGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGA
TACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTC
CCGAAGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCGA
GTCAGTGAGCGAGGAAGCGGAAGAGCGCCCAATACGCAAACCGCCTCTCCCCGCG
CGTTGGCCGATTCATTAATGCAGCTGGCACGACAGGTTTCCCGACTGGAAAGCGGG
CAGTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTTCA
CACAGGAAACAGCTATGACCATGATTACGCCAAGCGGGCCCGACGTCGCATGCTCC
ATTGCATACGTTGTATCCATATCATAATATGTACATTTATATTGGCTCATGTCCAACAT
TACCGCCATGTTGACATTGATTATTGACTAGTTATTAATAGTAATCAATTACGGGGTC
ATTAGTTCATAGCCCATATATGGAGTTCCGCGTTACATAACTTACGGTAAATGGCCC
GCCTGGCTGACCGCCCAACGACCCCCGCCCATTGACGTCAATAATGACGTATGTTC
CCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGG
GACTTTCCTACTTGGCAGTACATCTACGTATTAGTCATCGCTATTACCATGGTGATGC
GGTTTTGGCAGTACATCAATGGGCGTGGATAGCGGTTTGACTCACGGGGATTTCCA
AGTCTCCACCCCATTGACGTCAATGGGAGTTTGTTTTGGCACCAAAATCAACGGGAC
TTTCCAAAATGTCGTAACAACTCCGCCCCATTGACGCAAATGGGCGGTAGGCGTGT
ACGGTGGGAGGTCTATATAAGCAGAGCTCGTTTAGTGAACCGGGGTCTCTCTGGTT
AGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACT
CTGGTAACTAGAGATCCCTCAGACCCTTTTAGTCAGTGTGGAAAATCTCTAGCAGTG
GCGCCCGAACAGGGACTTGAAAGCGAAAGGGAAACCAGAGGAGCTCTCTCGACGC
AGGACTCGGCTTGCTGAAGCGCGCACGGCAAGAGGCGAGGGGCGGCGACTGGTG
AGTACGCCAAAAATTTTGACTAGCGGAGGCTAGAAGGAGAGAGATGGGTGCGAGAG
CGTCAGTATTAAGCGGGGGAGAATTAGATCGCGATGGGAAAAAATTCGGTTAAGGC
CAGGGGGAAAGAAAAAATATAAATTAAAACATATAGTATGGGCAAGCAGGGAGCTAG
AACGATTCGCAGTTAATCCTGGCCTGTTAGAAACATCAGAAGGCTGTAGACAAATAC
TGGGACAGCTACAACCATCCCTTCAGACAGGATCAGAAGAACTTAGATCATTATATA
ATACAGTAGCAACCCTCTATTGTGTGCATCAAAGGATAGAGATAAAAGACACCAAGG
AAGCTTTAGACAAGATAGAGGAAGAGCAAAACAAAAGTAAGACCACCGCACAGCAA
GCGGCCGCTGATCTTCAGACCTGGAGGAGGAGATATGAGGGACAATTGGAGAAGT
GAATTATATAAATATAAAGTAGTAAAAATTGAACCATTAGGAGTAGCACCCACCAAGG
CAAAGAGAAGAGTGGTGCAGAGAGAAAAAAGAGCAGTGGGAATAGGAGCTTTGTTC
CTTGGGTTCTTGGGAGCAGCAGGAAGCACTATGGGCGCAGCCTCAATGACGCTGA
CGGTACAGGCCAGACAATTATTGTCTGGTATAGTGCAGCAGCAGAACAATTTGCTGA
GGGCTATTGAGGCGCAACAGCATCTGTTGCAACTCACAGTCTGGGGCATCAAGCAG
CTCCAGGCAAGAATCCTGGCTGTGGAAAGATACCTAAAGGATCAACAGCTCCTGGG
GATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGA
CAGAGAAATTAACAATTACACAAGCTTAATACACTCCTTAATTGAAGAATCGCAAAAC
CAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAG
GAGGCTTGGTAGGTTTAAGAATAGTTTTTGCTGTACTTTCTATAGTGAATAGAGTTAG
GCAGGGATATTCACCATTATCGTTTCAGACCCACCTCCCAACCCCGAGGGGACCCG
ACAGGCCCGAAGGAATAGAAGAAGAAGGTGGAGAGAGAGACAGAGACAGATCCAT
TCGATTAGTGAACGGATCTCGACGGTATCGGTTAACTTTTAAAAGAAAAGGGGGGAT
TGGGGGGTACAGTGCAGGGGAAAGAATAGTAGACATAATAGCAACAGACATACAAA
CTAAAGAATTACAAAAACAAATTACAAAAATTCAAAATTTTATCGATCACGAGACTAG
CCTCGAGCTAGCGGTACCGATCAAGGTTAGGAACAGAGAGACAGGAGAATATGGG
CCAAACAGGATATCTGTGGTAAGCAGTTCCTGCCCCGGCTCAGGGCCAAGAACAGT
TGGAACAGCAGAATATGGGCCAAACAGGATATCTGTGGTAAGCAGTTCCTGCCCCG
GCTCAGGGCCAAGAACAGATGGTCCCCAGATGCGGTCCCGCCCTCAGCAGTTTCTA
GAGAACCATCAGATGTTTCCAGGGTGCCCCAAGGACCTGAAATGACCCTGTGCCTT
ATTTGAACTAACCAATCAGTTCGCTTCTCGCTTCTGTTCGCGCGCTTCTGCTCCCCG
AGCTCAATAAAAGAGCCCACAACCCCTCACTCGGCGCGCCAGTCCTCCGATAGACT
GCGTCGCCCGGGATCCACCGGTGCCACCATGGAGCCTCTGCGGCTGCTGATCCTG
CTGTTCGTGACCGAGCTGTCCGGCGCCCACAACACCACAGTGTTTCAGGGAGTGG
CAGGACAGTCCCTGCAGGTGTCTTGCCCATACGACTCTATGAAGCACTGGGGCCGG
AGAAAGGCATGGTGCAGGCAGCTGGGAGAGAAGGGACCATGTCAGCGCGTGGTGT
CTACACACAACCTGTGGCTGCTGAGCTTCCTGAGGCGCTGGAATGGCTCCACAGCC
ATCACCGACGATACACTGGGCGGCACCCTGACAATCACCCTGAGGAATCTGCAGCC
ACACGACGCCGGCCTGTATCAGTGTCAGAGCCTGCACGGCTCCGAGGCAGATACC
CTGCGGAAGGTGCTGGTGGAGGTGCTGGCCGACCCCCTGGATCACAGAGACGCAG
GCGATCTGTGGTTCCCTGGCGAGAGCGAGTCCTTTGAGGATGCCCACGTGGAGCA
CTCTATCAGCCGGGCCGAGAGACACGTGAAGGAGGACGATGGAAGGAAGTCTCCT
GGAGAGGTGCCACCTGGAACCAGCCCAGCATGCATCCTGGCAACATGGCCACCAG
GCCTGCTGGTGCTGCTGTGGCAGGAGACAACACTGCCCGAGCACTGTTTTTCCTGG
ACCCTGGAGGCCGGCACAGGCTGATGAGTCGACGAATTCGAGCATCTTACCGCCAT
TTATTCCCATATTTGTTCTGTTTTTCTTGATTTGGGTATACATTTAAATGTTAATAAAAC
AAAATGGTGGGGCAATCATTTACATTTTTAGGGATATGTAATTACTAGTTCAGGTGTA
TTGCCACAAGACAAACATGTTAAGAAACTTTCCCGTTATTTACGCTCTGTTCCTGTTA
ATCAACCTCTGGATTACAAAATTTGTGAAAGATTGACTGATATTCTTAACTATGTTGC
TCCTTTTACGCTGTGTGGATATGCTGCTTTAATGCCTCTGTATCATGCTATTGCTTCC
CGTACGGCTTTCGTTTTCTCCTCCTTGTATAAATCCTGGTTGCTGTCTCTTTATGAGG
AGTTGTGGCCCGTTGTCCGTCAACGTGGCGTGGTGTGCTCTGTGTTTGCTGACGCA
ACCCCCACTGGCTGGGGCATTGCCACCACCTGTCAACTCCTTTCTGGGACTTTCGC
TTTCCCCCTCCCGATCGCCACGGCAGAACTCATCGCCGCCTGCCTTGCCCGCTGCT
GGACAGGGGCTAGGTTGCTGGGCACTGATAATTCCGTGGTGTTGTCGGGGAAGCT
GACGTCCTTTCGAATTCGGTACCTTTAAGACCAATGACTTACAAGGCAGCTGTAGAT
CTTAGCCACTTTTTAAAAGAAAAGGGGGGACTGGAAGGGCTAATTCACTCCCAACGA
AGACAAGATCTGCTTTTTGCTTGTACTGGGTCTCTCTGGTTAGACCAGATCTGAGCC
TGGGAGCTCTCTGGCTAACTAGGGAACCCACTGCTTAAGCCTCAATAAAGCTTGCCT
TGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTAGTAGTTCATGTCATCTTA
TTATTCAGTATTTATAACTTGCAAAGAAATGAATATCAGAGAGTGAGAGGA

In some embodiments, the TREM2-encoding expression construct has a nucleic acid sequence of SEQ ID NO. 15 or is a variant thereof having at least 85% (e.g. at least 86%, 90%, 95%, 96%, 97%, 98%, 99%, or more) sequence identity to the nucleic acid sequence of SEQ ID NO. 15. In some embodiments, the TREM2-encoding expression construct has at least 90% (e.g. at least 91%, 95%, 96%, 97%, 98%, 99%, or more) sequence identity to the nucleic acid sequence of SEQ ID NO. 15. In some embodiments, the TREM2-encoding expression construct has at least 95% (e.g. at least 96%, 97%, 98%, 99%, or more) sequence identity to the nucleic acid sequence of SEQ ID NO. 15. In some embodiments, the TREM2-encoding expression construct has at least 98% (e.g. at least 99% or more) sequence identity to the nucleic acid sequence of SEQ ID NO. 15. In some embodiments, the TREM2-encoding expression construct has at least 99% sequence identity to the nucleic acid sequence of SEQ ID NO. 15. In some embodiments, the TREM2-encoding expression construct has the nucleic acid sequence of SEQ ID NO. 15.

Another exemplary high expression constitutive promoter suitable for use in conjunction with the disclosed methods and compositions is a integrin subunit alpha M (CD11b) promoter, which is the native promoter of the CD11b gene. The CD11b promoter may have a sequence of SEQ ID NO. 17 or may be a variant thereof having at least 85% (e.g., at least 86%, 90%, 95%, 96%, 97%, 98%, 99%, or more) sequence identity to the nucleic acid sequence of SEQ ID NO. 17, as is shown below.

(SEQ ID NO. 17)
ATTTTTTTGTAGAGACAGGGTCTCTCTATGTTGCCCAGGCTGGTTTCAA
ACTCCCAGGCTCAAGCAATCCTCCTGCCTTGGCCTCCCAAAGTGCTGGC
ATTACAGGCGTGAGCCACTGCGCCTGGCCCGTATTAATGTTTAGAACAC
GAATTCCAGGAGGCAGGCTAAGTCTGTTCAGCTTGTTCATATGCTTGGG
CCAACCCAAGAAACAAGTGGGTGACAAATGGCACCTTTTGGATAGTGGT
ATTGACTTTGAAAGTTTGGGTCAGGAAGCTGGGGAGGAAGGGTGGGCAG
GCTGTGGGCAGTCCTGGGCGGAAGACCAGGCAGGGCTATGTGCTCACTG
AGCCTCCGCCCTCTTCCTTTGAATCTCTGATAGACTTCTGCCTCCTACT
TCTCCTTTTCTGCCCTTCTTTGCTTTGGTGGCTTCCTTGTGGTTCCTCA
GTGGTGCCTGCAACCCCTGGTTCACCTCCTTCCAGGTTCTGG

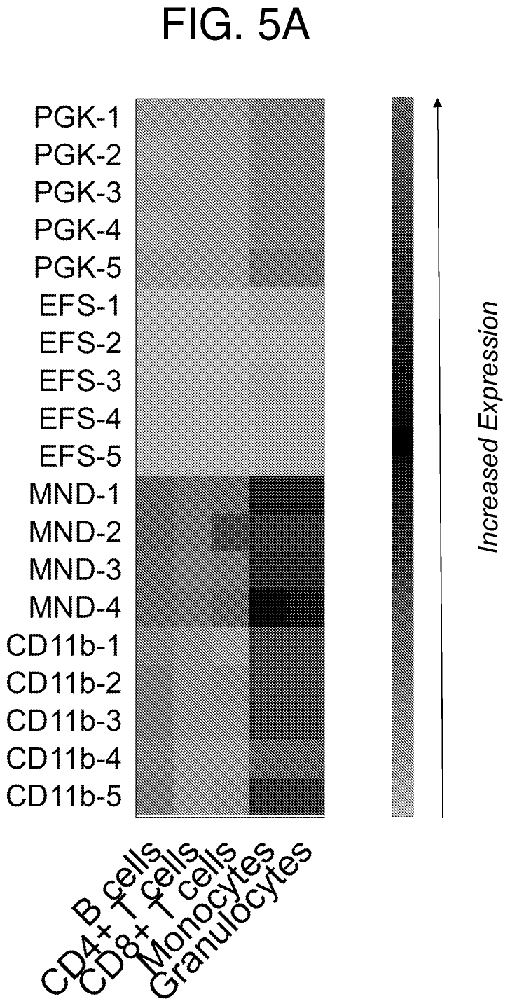

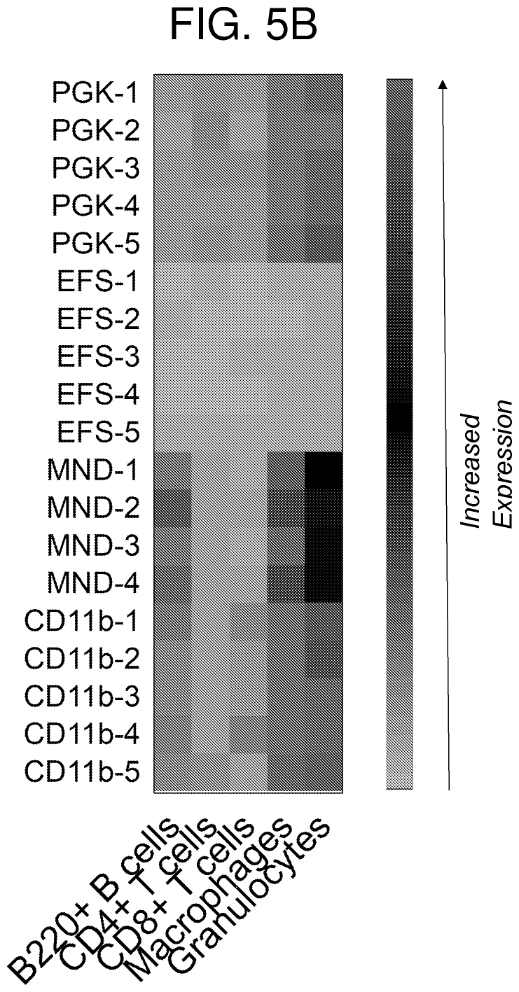

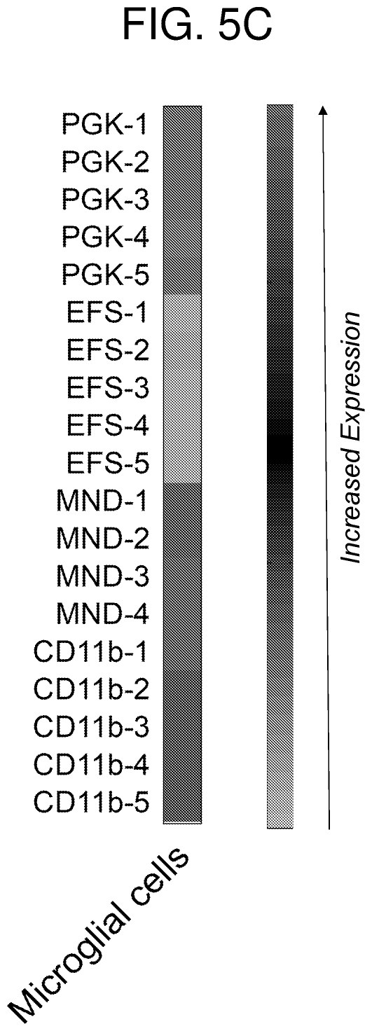

The present inventors have demonstrated that an expression vector containing a transgene operably linked to a CD11b promoter was able to selectively drive transgene expression selectively in cells of the myeloid lineage in vivo at levels that were substantially higher than the expression levels observed in cells of the lymphoid lineage (i.e., T-cells and B-cells; see Example 6 and FIG. 5). Accordingly, the present disclosure contemplates expression constructs encoding a TREM2 protein (e.g., a TREM2 protein having any one of the amino acid sequences of SEQ ID NOs. 1-3 or a variant thereof having at least 85% (e.g., at least 86%, 90%, 95%, 96%, 97%, 98%, 99%, or more) sequence identity to the nucleic acid sequence of SEQ ID NOs. 1-3 operably linked to a CD11b promoter (e.g., having the nucleic acid sequence of SEQ ID NO. 17 or a variant thereof having at least 85% (e.g. at least 86%, 90%, 95%, 96%, 97%, 98%, 99%, or more) sequence identity to the nucleic acid sequence of SEQ ID NO. 17. In some embodiments, the CD11b promoter has at least 90% (e.g. at least 91%, 95%, 96%, 97%, 98%, 99%, or more) sequence identity to the nucleic acid sequence of SEQ ID NO. 17. In some embodiments, the CD11b promoter has at least 95% (e.g. at least 96%, 97%, 98%, 99%, or more) sequence identity to the nucleic acid sequence of SEQ ID NO. 17. In some embodiments, the CD11b promoter has at least 99% sequence identity to the nucleic acid sequence of SEQ ID NO. 17. In some embodiments, the CD11b promoter has the nucleic acid sequence of SEQ ID NO. 17.

An exemplary expression vector construct containing a codon-optimized TREM2-encoding polynucleotide (SEQ ID NO. 8) operably linked to a CD11b promoter (SEQ ID NO. 17) is provided in SEQ ID NO. 18, as is shown below. The codon-optimized TREM2-encoding polynucleotide of SEQ ID NO. 8 encodes a wild-type TREM2 peptide of SEQ ID NO. 1.

(SEQ ID NO. 18)
ATTTTTTTGTAGAGACAGGGTCTCTCTATGTTGCCCAGGCTGGTTTCAA
ACTCCCAGGCTCAAGCAATCCTCCTGCCTTGGCCTCCCAAAGTGCTGGC
ATTACAGGCGTGAGCCACTGCGCCTGGCCCGTATTAATGTTTAGAACAC
GAATTCCAGGAGGCAGGCTAAGTCTGTTCAGCTTGTTCATATGCTTGGG
CCAACCCAAGAAACAAGTGGGTGACAAATGGCACCTTTTGGATAGTGGT
ATTGACTTTGAAAGTTTGGGTCAGGAAGCTGGGGAGGAAGGGTGGGCAG
GCTGTGGGCAGTCCTGGGCGGAAGACCAGGCAGGGCTATGTGCTCACTG
AGCCTCCGCCCTCTTCCTTTGAATCTCTGATAGACTTCTGCCTCCTACT
TCTCCTTTTCTGCCCTTCTTTGCTTTGGTGGCTTCCTTGTGGTTCCTCA
GTGGTGCCTGCAACCCCTGGTTCACCTCCTTCCAGGTTCTGGGGATCCA
CCGGTATGGAGCCTCTGAGACTGCTGATTCTGCTGTTTGTCACTGAACT
GAGCGGCGCACATAATACCACTGTCTTCCAGGGCGTCGCTGGGCAGTCT
CTGCAGGTGAGCTGCCCCTACGACTCTATGAAGCACTGGGGCCGGAGAA
AGGCATGGTGCCGGCAGCTGGGAGAGAAGGGACCTTGTCAGAGAGTGGT
GAGCACCCACAACCTGTGGCTGCTGTCCTTCCTGAGGCGCTGGAATGGC
TCTACAGCCATCACCGACGATACACTGGGCGGCACCCTGACAATCACCC
TGAGGAACCTGCAGCCTCACGACGCAGGCCTGTATCAGTGCCAGTCCCT
GCACGGCTCTGAGGCCGATACACTGAGGAAGGTGCTGGTGGAGGTGCTG
GCCGACCCTCTGGATCACAGGGACGCAGGCGATCTGTGGTTCCCAGGCG
AGAGCGAGTCCTTTGAGGATGCCCACGTGGAGCACTCTATCAGCCGGTC
CCTGCTGGAGGGAGAGATCCCATTCCCCCCTACCAGCATCCTGCTGCTG
CTGGCCTGTATCTTTCTGATCAAGATCCTGGCAGCATCCGCCCTGTGGG
CAGCAGCCTGGCACGGACAGAAGCCAGGAACACACCCACCATCCGAGCT
GGATTGCGGACATGACCCCGGCTACCAGCTGCAGACACTGCCTGGCCTG
AGGGATACATGATGA

In some embodiments, the TREM2-encoding expression construct has a nucleic acid sequence of SEQ ID NO. 18 or is a variant thereof having at least 85% (e.g. at least 86%, 90%, 95%, 96%, 97%, 98%, 99%, or more) sequence identity to the nucleic acid sequence of SEQ ID NO. 18. In some embodiments, the TREM2-encoding expression construct has at least 90% (e.g. at least 91%, 95%, 96%, 97%, 98%, 99%, or more) sequence identity to the nucleic acid sequence of SEQ ID NO. 18. In some embodiments, the TREM2-encoding expression construct has at least 95% (e.g. at least 96%, 97%, 98%, 99%, or more) sequence identity to the nucleic acid sequence of SEQ ID NO. 18. In some embodiments, the TREM2-encoding expression construct has at least 98% (e.g. at least 99% or more) sequence identity to the nucleic acid sequence of SEQ ID NO. 18. In some embodiments, the TREM2-encoding expression construct has at least 99% sequence identity to the nucleic acid sequence of SEQ ID NO. 18. In some embodiments, the TREM2-encoding expression construct has the nucleic acid sequence of SEQ ID NO. 18.

Accordingly, the present disclosure contemplates the use of an MND or CD11b promoter to selectively drive TREM2 transgene expression in a population of cells (e.g., pluripotent cells, ESCs, iPSCs, multipotent cells, CD34+ cells, HSCs, myeloid cells (e.g., MPCs), BLPCs, monocytes, macrophages, microglial progenitor cells, or microglia). In some embodiments, upon administration of a population of cells expressing a TREM2 transgene to a subject, the MND promoter drives higher TREM2 transgene expression in cells of the myeloid lineage (e.g., macrophages, MPCs, mast cells, erythrocytes, megakaryocytes, thrombocytes, myeloblasts, basophils, neutrophils, eosinophils, or monocytes) than in cells of the lymphoid lineage (e.g., natural killer (NK) cells, T-cells, or B-cells). In some embodiments, upon administration of the cells to the subject, expression of the TREM2 transgene is higher in myeloid cells in the subject by at least 50% (e.g., at least 50%, 55%, 60%, 65%, 70%, 75%, 80%, 85%, 90%, 95%, 96%, 97%, 98%, 99%, 100%, 150%, 200%, 250%, 300%, 350%, 400%, 450%, 500%, or more) than in lymphoid cells in the subject.

In some embodiments, the lymphoid cells are T-cells. In some embodiments, the lymphoid cells are B-cells. In some embodiments, the lymphoid cells are natural killer (NK) cells. In some embodiments, the lymphoid cells are T-cells and B-cells.

In some embodiments, the expression level of the TREM2 transgene is measured in one or more organs, tissues, or body fluids of a subject. In some embodiments, the one or more body fluids is peripheral blood.

### Host Cells

Cells that may be used in conjunction with the compositions and methods described herein include cells that are capable of undergoing further differentiation (e.g., pluripotent cells, ESCs, iPSCs, CD34+ cells, HSCs, MPCs, BLPCs, monocytes, or microglial progenitor cells) or differentiated cells (e.g., macrophages or microglia). For example, one type of cell that can be used in conjunction with the compositions and methods described herein is a pluripotent cell. A pluripotent cell is a cell that possesses the ability to develop into more than one differentiated cell type. Examples of pluripotent cells are ESCs and iPSCs. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system. Another type of cell that can be used in conjunction with the compositions and methods described herein is a multipotent cell. A multipotent cell is a cell that possesses the ability to differentiate into multiple, but not all cell types. A non-limiting example of a multipotent cell is a CD34+ cell (e.g., HSCs or MPC).

Cells that may be used in conjunction with the compositions and methods described herein include HSCs and MPCs. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cell types.

Cells that may be used in conjunction with the compositions and methods described herein include allogeneic cells and autologous cells. All of the aforementioned cell types are capable of differentiating into microglia. Cells described herein may also differentiate into microglial progenitors or microglial stem cells. Differentiation may occur ex vivo or in vivo. Methods for ex vivo differentiation of human ESCs and iPSCs are known by those of skill in the art and are described in Muffat et al., Nature Medicine 22:1358-1367 (2016) and Pandya et al., Nature Neuroscience (2017) epub ahead of print, the disclosures of which are incorporated herein by reference as they pertain to methods of differentiating pluripotent cells into microglia.

### Microglia

Cells that may be used in conjunction with the compositions and methods described herein include those that are capable of differentiating into microglial cells or cells that are differentiated microglial cells. Microglia are myeloid-derived cells that serve as the immune cells, or resident macrophages, of the central nervous system. Microglia are highly similar to macrophages, both genetically and functionally, and share the ability dynamically exhibit pro-inflammatory and anti-inflammatory states. Microglia can be polarized into pro-inflammatory and anti-inflammatory phenotypes by extracellular signals, e.g., signals from neighboring neurons or astrocytes, cell debris, toxins, infection, ischemia, and traumatic injury, among others. Microglia expressing a pro-inflammatory signature are often observed in the diseased brain, particularly in diseases involving neuroinflammation, such as AD. Microglia with pro-inflammatory phenotypes have also been observed in mouse models of AD, such as the double transgenic APP/PS1 mouse. It is unclear whether pro-inflammatory microglia are a cause or consequence of neuroinflammation, but once microglia are classically activated, they can secrete pro-inflammatory cytokines, e.g., TNF-α, IL-1p, and IL-6, chemokines, and nitric oxide, which can lead to sustained inflammation, neuronal damage, and further activation of microglia. This positive feedback loop can be harmful to brain tissue; therefore, methods of reducing microglial pro-inflammatory signaling and/or anti-inflammatory signaling in microglia may help subjects with diseases featuring neuroinflammation, such as, e.g., AD, PLOSL, FTLD, or PD.

### Expression of TREM2 in Mammalian Cells

TREM2 activity is reduced in some patients with AD or PLOSL, and AD brains contain microglia expressing a pro-inflammatory phenotype. Additionally, microglia from PLOSL patients appear to have delayed but enhanced inflammatory responses compared to healthy controls. The compositions and methods described herein target these dysfunctions by administering cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding TREM2 (e.g., a transgene capable of expression in macrophages or microglial cells). In order to utilize these agents for therapeutic application in the treatment of an NCD, these agents can be directed to the interior of the cell, and in particular examples, to particular organelles or the plasma membrane. A wide array of methods has been established for the delivery of such proteins to mammalian cells and for the stable expression of genes encoding such proteins in mammalian cells.

### Polynucleotides Encoding TREM2

One platform that can be used to achieve therapeutically effective intracellular concentrations of TREM2 in mammalian cells (e.g., pluripotent cells, ESCs, iPSCs, multipotent cells, CD34+ cells, HSCs, MPCs, BLPCs, monocytes, macrophages, microglial progenitor cells, or microglia) is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

TREM2 can also be introduced into a mammalian cell by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

Recognition and binding of the polynucleotide encoding TREM2 by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a high expression constitutive promoter, such as, e.g., an MND promoter (such as, e.g., an MND promoter having a nucleic acid sequence of SEQ ID NO. 14 or SEQ ID NO. 16 or a variant thereof having at least 85% (e.g., at least 86%, 90%, 95%, 96%, 97%, 98%, 99%, or more) sequence identity to the nucleic acid sequence of SEQ ID NO. 14 or SEQ ID NO. 16) or a CD11b promoter (such as, e.g., a CD11b promoter having a nucleic acid sequence of SEQ ID NO. 17 or a variant thereof having at least 85% (e.g., at least 86%, 90%, 95%, 96%, 97%, 98%, 99%, or more) sequence identity to the nucleic acid sequence of SEQ ID NO. 17) the sequence of which can be recognized and bound by specific transcription initiation factors and ultimately RNA polymerase.

Once a polynucleotide encoding TREM2 has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the promoter and thus regulates gene expression. The chemical reagent can serve to facilitate the binding of RNA polymerase and/or transcription factors to the promoter, e.g., by removing a repressor protein that has bound the promoter. Alternatively, the chemical reagent can serve to enhance the affinity of the promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode TREM2 and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding a water-forming NADH oxidase, for example, at a position 5′ or 3′ to this gene. In a preferred orientation, the enhancer is positioned at the 5′ side of the promoter, which in turn is located 5′ relative to the polynucleotide encoding TREM2.

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous microRNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRTs for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia) (Gentner et al., Science Translational Medicine. 2:58ra34 (2010)). A miR-126 targeting sequence, for example, incorporated within a transgene would allow for targeted expression of the transgene in cells of the myeloid lineage and repressed expression in HSPCs and other progenitor cells, thus minimizing off-target cytotoxic effects. In some embodiments, the transgene encoding TREM2 agent may include a miR-126 targeting sequence.

### Signal Peptides

Polynucleotides encoding TREM2 may include one or more polynucleotides encoding a signal peptide. Signal peptides may have amino acid sequences of 16-30 residues in length, and may be located upstream of (i.e., 5′ to) a polynucleotide encoding TREM2. These signal peptides allow for the recognition of the nascent polypeptides during synthesis by signal recognition particles resulting in translocation to the ER, packaging into transport vesicles, and translocation to a target cellular compartment, to the lipid membrane, or to the extracellular space. Exemplary signal peptides for protein translocation are those from TREM2, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding TREM2 may be utilized as a therapeutic strategy to correct a protein deficiency (e.g., TREM2) by infusing the missing protein into the bloodstream. As the blood perfuses patient tissues, TREM2 is taken up by cells and transported to its site of action.

### ApoE Tag for Blood-Brain Barrier Penetrance of Secreted TREM2b

In some embodiments, the TREM2 (e.g., TREM2 fusion protein) is modified to penetrate the blood-brain barrier (BBB). Modifications for mediating BBB penetrance are well known in the art. Exemplary modifications are the use of tags containing the Rb domain (amino acid residues 148-173 of SEQ ID NO. 13) of ApoE. The complete ApoE peptide sequence is shown below.

(SEQ ID NO. 13)
MKVLWAALLVTFLAGCQAKVEQAVETEPEPELRQQTEWQSGQRWELALG
RFWDYLRWVQTLSEQVQEELLSSQVTQELRALMDETMKELKAYKSELEE
QLTPVAEETRARLSKELQAAQARLGADMEDVCGRLVQYRGEVQAMLGQS
TEELRVRLASHLRKLRKRLLRDADDLQKRLAVYQAGAREGAERGLSAIR
ERLGPLVEQGRVRAATVGSLAGQPLQERAQAWGERLRARMEEMGSRTRD
RLDEVKEQVAEVRAKLEEQAQQIRLQAEAFQARLKSWFEPLVEDMQRQW
AGLVEKVQAAVGTSAAPVPSDNH

ApoE is an important protein involved in lipid transport, and its cellular internalization is mediated by several members of the low-density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a potent Rb peptide derived from ApoE, has the ability to translocate protein across the BBB into the brain when engineered as fusion proteins. This method can therefore function to selectively open the BBB for therapeutic agents (e.g., soluble TREM2) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, the TREM2 fusion protein has a peptide sequence containing the LDLRf Rb domain of SEQ ID NO. 13, or a fragment, variant, or oligomer thereof. An exemplary receptor-binding domain can be found in the N-terminus of ApoE, for example, between amino acid residues 1 to 191 of SEQ ID NO. 13, between amino acid residues 25 to 185 of SEQ ID NO. 13, between amino acid residues 50 to 180 of SEQ ID NO. 13, between amino acid residues 75 to 175 of SEQ ID NO. 13, between amino acid residues 100 to 170 of SEQ ID NO. 13, or between amino acid residues 125 to 165 of SEQ ID NO. 13. An exemplary receptor-binding domain has the amino acid sequence of residues 159 to 167 of SEQ ID NO. 13.

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining some degree of the biochemical activity for transport across the BBB.

### Vectors for the Expression of TREM2

In addition to achieving high rates of transcription and translation, stable expression of an exogenous gene in a mammalian cell (e.g., pluripotent cell, ESC, iPSC, multipotent cell, CD34+ cell, HSC, MPC, BLPC, monocyte, macrophage, microglial progenitor cell, or microglial cell) can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide that encodes TREM2, as well as, e.g., additional sequence elements used for the expression of these agents and/or the integration of these polynucleotides into the genome of a mammalian cell. Certain vectors that can be used for the expression of TREM2 include plasmids that contain regulatory sequences, such as promoter and enhancer regions, which direct gene transcription. Other useful vectors for expression of TREM2 contain polynucleotides that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Viral Vectors for Expression of TREM2

Viral genomes provide a rich source of vectors that can be used for the efficient delivery of exogenous genes into a mammalian cell (e.g., pluripotent cell, ESC, iPSC, multipotent cell, CD34+ cell, HSC, MPC, BLPC, monocyte, macrophage, microglial progenitor cell, or microglial cell). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

In some embodiments, the viral vector includes a polynucleotide having at least 85% (e.g. at least 86%, 90%, 95%, 96%, 97%, 98%, 99%, or more) sequence identity to the nucleic acid sequence of SEQ ID NO. 15. In some embodiments, the viral vector includes a polynucleotide having at least 90% (e.g. at least 91%, 95%, 96%, 97%, 98%, 99%, or more) sequence identity to the nucleic acid sequence of SEQ ID NO. 15. In some embodiments, the viral vector includes a polynucleotide having at least 95% (e.g. at least 96%, 97%, 98%, 99%, or more) sequence identity to the nucleic acid sequence of SEQ ID NO. 15. In some embodiments, the viral vector includes a polynucleotide having at least 98% (e.g. at least 99% or more) sequence identity to the nucleic acid sequence of SEQ ID NO. 15. In some embodiments, the viral vector includes a polynucleotide having at least 99% sequence identity to the nucleic acid sequence of SEQ ID NO. 15. In some embodiments, the viral vector includes a polynucleotide having the nucleic acid sequence of SEQ ID NO. 15.

In some embodiments, the viral vector includes a polynucleotide having at least 85% (e.g. at least 86%, 90%, 95%, 96%, 97%, 98%, 99%, or more) sequence identity to the nucleic acid sequence of SEQ ID NO. 18. In some embodiments, the viral vector includes a polynucleotide having at least 90% (e.g. at least 91%, 95%, 96%, 97%, 98%, 99%, or more) sequence identity to the nucleic acid sequence of SEQ ID NO. 18. In some embodiments, the viral vector includes a polynucleotide having at least 95% (e.g. at least 96%, 97%, 98%, 99%, or more) sequence identity to the nucleic acid sequence of SEQ ID NO. 18. In some embodiments, the viral vector includes a polynucleotide having at least 98% (e.g. at least 99% or more) sequence identity to the nucleic acid sequence of SEQ ID NO. 18. In some embodiments, the viral vector includes a polynucleotide having at least 99% sequence identity to the nucleic acid sequence of SEQ ID NO. 18. In some embodiments, the viral vector includes a polynucleotide having the nucleic acid sequence of SEQ ID NO. 18.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Viral Regulatory Elements

The viral regulatory elements are components of delivery vehicles used to introduce nucleic acid molecules into a host cell (e.g., pluripotent cells, ESC, iPSC, multipotent cell, CD34+ cell, HSC, MPC, BLPC, monocyte, macrophage, microglial progenitor cell, or microglial cell). The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. One skilled in the art would also appreciate that as other viral regulatory elements are identified, these may be used with the nucleic acid molecules described herein.

### Adeno-Associated Viral Vectors for Nucleic Acid Delivery

Nucleic acids of the compositions and methods described herein may be incorporated into rAAV vectors and/or virions in order to facilitate their introduction into a cell (e.g., pluripotent cells, ESC, iPSC, multipotent cell, CD34+ cell, HSC, MPC, BLPC, monocyte, macrophage, microglial progenitor cell, or microglial cell). AAV vectors can be used in the central nervous system, and appropriate serotypes are discussed in Pignataro et al., J Neural Transm. (2017), epub ahead of print, the disclosure of which is incorporated herein by reference as it pertains to AAV serotypes useful in CNS gene therapy. rAAV vectors useful in the compositions and methods described herein are recombinant nucleic acid constructs that include (1) a heterologous sequence to be expressed (e.g., a polynucleotide encoding TREM2) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet. 25:436 (2000) and Kolman and Stemmer, Nat. Biotechnol. 19:423 (2001).

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell e.g., pluripotent cells, ESC, iPSC, multipotent cell, CD34+ cell, HSC, MPC, BLPC, monocyte, macrophage, microglial progenitor cell, or microglial cell) are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

### Disruption of Endogenous TREM2

In some embodiments, endogenous TREM2 is disrupted (e.g., in a subject undergoing treatment, such as in a population of neurons in a subject undergoing treatment, or in the cells to be administered to the subject). Exemplary methods for disrupting endogenous TREM2 expression are those in which an inhibitory RNA molecule is administered to the subject or contacted with a population of neurons in the subject or the population of cells to be administered to the subject. The inhibitory RNA molecule may function to disrupt endogenous TREM2 expression, for example, act by way of the RNA interference (RNAi) pathway. An inhibitory RNA molecule can decrease the expression level (e.g., protein level or mRNA level) of endogenous TREM2. For example, an inhibitory RNA molecule includes a short interfering RNA, short hairpin RNA, and/or a miRNA that targets full-length endogenous TREM2. A siRNA is a double-stranded RNA molecule that typically has a length of about 19-25 base pairs. A shRNA is an RNA molecule including a hairpin turn that decreases expression of target genes via RNAi. shRNAs can be delivered to cells in the form of plasmids, e.g., viral or bacterial vectors, e.g., by transfection, electroporation, or transduction). A miRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability or decrease immunogenicity.

In some embodiments, the inhibitory RNA molecule decreases the level and/or activity or function of endogenous TREM2. In embodiments, the inhibitory RNA molecule inhibits expression of endogenous TREM2. In other embodiments, the inhibitor RNA molecule increases degradation of endogenous TREM2 and/or decreases the stability of endogenous TREM2. The inhibitory RNA molecule can be chemically synthesized or transcribed in vitro.

In some embodiments, the endogenous TREM2 is disrupted in the cells containing the TREM2 transgene using, for example, the gene editing techniques described herein. In some embodiments, the endogenous TREM2 is globally disrupted in the subject using, for example, the gene editing techniques described herein. In some embodiments, the endogenous TREM2 is disrupted in a population of neurons in the subject using, for example, the gene editing techniques described herein. In some embodiments, disruption of endogenous TREM2 in the subject, neurons, and/or cells containing the TREM2 transgene occurs prior to administration of the cells to the subject.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

### Nuclease-Mediated Gene Regulation

Another useful tool for the disruption and/or integration of target genes into the genome of a cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and a CRISPR-associated protein (Cas; e.g., Cas9 or Cas12a). This ensemble of DNA and protein directs site specific DNA cleavage of a target sequence by first incorporating foreign DNA into CRISPR loci.

Polynucleotides containing these foreign sequences and the repeat-spacer elements of the CRISPR locus are in turn transcribed in a host cell to create a guide RNA, which can subsequently anneal to a target sequence and localize the Cas nuclease to this site. In this manner, highly site-specific Cas-mediated DNA cleavage can be engendered in a foreign polynucleotide because the interaction that brings Cas within close proximity of the target DNA molecule is governed by RNA: DNA hybridization. As a result, one can theoretically design a CRISPR/Cas system to cleave any target DNA molecule of interest (e.g., endogenous TREM2). This technique has been exploited in order to edit eukaryotic genomes (Hwang et al. Nature Biotechnology 31:227 (2013), the disclosure of which is incorporated herein by reference) and can be used as an efficient means of site-specifically editing cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference. Alternative methods for disruption of a target DNS by site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference. In some embodiments, the endogenous TREM2 may be disrupted in the cells containing the TREM2 transgene using these gene editing techniques described herein.

### Transposon-Mediated Gene Regulation

In addition to viral vectors, a variety of additional tools have been developed that can be used for the incorporation of exogenous genes into cells (e.g., pluripotent cells, ESCs, iPSCs, multipotent cells, CD34+ cells, HSCs, MPCs, BLPCs, monocytes, macrophages, microglial progenitor cells, or microglia). One such method that can be used for incorporating polynucleotides encoding target genes into cells involves the use of transposons. Transposons are polynucleotides that encode transposase enzymes and contain a polynucleotide or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US 2005/0112764), the disclosures of each of which are incorporated herein by reference.

### Methods of Diagnosis

Subjects may be diagnosed as having an NCD (e.g., AD, PLOSL, FTLD, or PD) using methods well-known in the art, such as, e.g., the methods described in The Diagnostic and Statistical Manual of Mental Disorders, Fifth Edition and the International Classification of Diseases, 11^{th} Revision. For example, diagnosis of NCDs in a subject may be guided by neuropsychological testing to assess the degree of cognitive impairment in a subject. The subject's cognitive function may be assessed by performing cognitive tests that evaluate performance across one or more cognitive domains including but not limited to complex attention, executive function, learning and memory, language, perceptual-motor function, and social cognition. Comparison of cognitive function in the subject relative to a norm appropriate for the subjects age, medical history, education, socioeconomic status, and lifestyle (e.g., a reference population, such as, e.g., a general population) may be done to determine the diagnosis with respect to an NCD in the subject. The subject may be diagnosed as having a major NCD or a mild NCD. Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major NCD can be characterized by a score obtained on a cognitive test by a subject that is more than two standard deviations away from the mean score of a reference population (e.g., the mean score of a general population) or a score that is in the third percentile of the distribution of scores of the reference population. Mild NCD can be characterized by a score obtained on a cognitive test by a subject that is between one to two standard deviations away from the mean score of a reference population (e.g., the mean score of a general population) or a score that is between the 3^{rd} and 16^{th} percentile of the distribution of scores of the reference population. Non-limiting examples of cognitive tests include Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). Additionally or alternatively, the use of F18-fluorodeoxyglucose PET scans or MRI scans may be used to determine the presence of neurodegeneration in a subject with an NCD.

Furthermore, the subject may be tested for the presence of biomarkers specific to the particular NCD of interest. For example, a subject may be tested for the presence of biomarkers that indicate that the subject has AD, such as the presence of Aβ plaques or NFTs of hyperphosphorylated tau proteins in the forebrain of the subject, presence of mutations in the APP, PSEN1, PSEN2, and/or TREM2 genes in the subject, as well as variations in the 4 allele of APOE. A subject may also be tested for the presence of lipid-laden macrophages, presence of axonal spheroids, loss of axons and myelin, white matter degeneration, and/or mutations in the TREM2 gene to determine whether the subject has PLOSL. Furthermore, PLOSL patients are known to exhibit cystic bone lesions during the early disease stages, the presence of which may be used to guide the diagnosis of a patient with PLOSL.

### Methods of Treatment

### Selection of Subjects

Subjects that may be treated as described herein are subjects having or at risk of developing an NCD (e.g., AD, PLOSL, FTLD, or PD). The type of NCD may be TREM2-associated NCD (e.g., TREM2-associated AD, PLOSL, FTLD, or PD), sporadic NCD (e.g., sporadic AD, PLOSL, FTLD, or PD), NCD caused by an environmental factor, or NCD associated with a non-TREM2 mutation, e.g., a mutation in one or more of the genes associated with AD or PLOSL. The compositions and methods described herein can be used to treat subjects with normal TREM2 activity, reduced TREM2 activity, and subjects whose TREM2 mutational status and/or TREM2 activity level is unknown. The compositions and methods described herein may also be administered as a preventative treatment to subjects at risk of developing NCD, e.g., subjects with a TREM2 mutation, subjects with reduced TREM2 activity, subjects with a mutation in one or more of the genes associated with an NCD, or subjects exposed to an environmental toxin associated with NCD. Subjects at risk for an NCD may show early symptoms of NCD or may not yet be symptomatic when treatment is administered.

In some embodiments, the methods and compositions described herein may be administered to subjects with TREM2 mutations that include, for example, single amino acid substitutions (e.g., p.R47H, p.R62H, p.T66M, p.T66M, p.Y38C, p.T96K, p.D87N, p.H157Y, p.R98W, p.T96K, p.D87N, p.L211P, p.R136Q, or p.N68K). Additionally, the methods and compositions described herein may be administered to subjects with TREM2 mutations that include, for example single nucleotide substitutions or deletions (e.g., c.40G>T, c.C97>T, c.132G>A, c.267delGm c.313delG, c.377T>G, c.401A>G, c.482+2T>C, c.558GA). In some embodiments, the methods and compositions described herein may be administered to subjects carrying any other pathogenic mutation in the TREM2 gene. For example, pathogenic mutations in the TREM2 gene may be any of the mutations discussed in Guerreiro et al., The New England Journal of Medicine 368, 117-27, (2013), Jonsson et al., The New England Journal of Medicine, 368(2), 107-16, Ulrich et al., Neuron Review 94, 237:48, (2017), and Xing et al., Research and Reports in Biochemistry, 5, 89-100, (2015), the disclosures of which are incorporated herein by reference as they pertain to AD-associated or PLOSL-associated human TREM2 mutations.

### Routes of Administration

The cells and compositions described herein may be administered to a subject with an NCD (e.g., AD, PLOSL, FTLD, or PD) by a variety of routes, such as intracerebroventricularly, intrathecally, intraparenchymally, stereotactically, intravenously, intraosseously, or by means of a bone marrow transplant. In some embodiments, the cells and compositions described herein may be administered to a subject systemically (e.g., intravenously), directly to the central nervous system (CNS) (e.g., intracerebroventricularly, intrathecally, intraparenchymally, or stereotactically), or directly into the bone marrow (e.g., intraosseously). In some embodiments, the cells and compositions described herein are administered to a subject intracerebroventricularly into the cerebral lateral ventricles (a description of this method can be found in Capotondo et al., Science Advances 3:e1701211 (2017), incorporated herein by reference as it pertains to intracerebroventricular injection of hematopoietic stem and progenitor cells into the cerebral lateral ventricles of mouse models). The most suitable route for administration in any given case will depend on the particular cell or composition administered, the subject, pharmaceutical formulation methods, administration methods (e.g., administration time and administration route), the subject's age, body weight, sex, severity of the diseases being treated, the subject's diet, and the subject's excretion rate. Multiple routes of administration may be used to treat a single subject, e.g., intracerebroventricular or stereotactic injection and intravenous injection, intracerebroventricular or stereotactic injection and intraosseous injection, intracerebroventricular or stereotactic injection and bone marrow transplant, intracerebroventricular or stereotactic injection and intraparenchymal injection, intrathecal injection and intravenous injection, intrathecal injection and intraosseous injection, intrathecal injection and bone marrow transplant, intrathecal injection and intraparenchymal injection, intraparenchymal injection and intravenous injection, intraparenchymal injection and intraosseous injection, or intraparenchymal injection and bone marrow transplant. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Cells may be administered to a subject once, or cells may be administered one or more times (e.g., 2-10 times) per week, month, or year to a subject for treatment of an NCD.

### Conditioning

Prior to administration of cells (e.g., pluripotent cells, ESCs, iPSCs, multipotent cells, CD34+ cells, HSCs, MPCs, BLPCs, monocytes, macrophages, microglial progenitor cells, or microglia) or compositions, it may be advantageous to deplete or ablate endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as a-amanitin and derivatives thereof), agents capable of disrupting protein biosynthesis (e.g., agents that exhibit rRNA N-glycosidase activity, such as saporin and ricin A-chain), among others known in the art. Ablation may eliminate all microglia and/or hematopoietic stem and progenitor cells, or it may reduce microglia and/or hematopoietic stem and progenitor cells numbers by at least 5% (e.g., at least 5%, 10%, 20%, 30%, 40%, 50%, 60%, 70%, 80%, 90%, 95%, or more). One or more agents to ablate microglia and/or hematopoietic stem and progenitor cells may be administered at least one week (e.g., 1, 2, 3, 4, 5, or 6 weeks or more) before administration of the cells or compositions described herein. Cells administered in the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a subject a population of cells (e.g., pluripotent cells, ESCs, iPSCs, multipotent cells, CD34+ cells, HSCs, MPCs, BLPCs, monocytes, macrophages, microglial progenitor cells, or microglia). These cells may be cells that have not been modified to contain the transgene encoding TREM2 (e.g., a transgene capable of expression in macrophages or microglia). These cells may have disrupted endogenous TREM2. The cells may be administered systemically (e.g., intravenously), or by bone marrow transplantation to reconstitute the bone marrow compartment following conditioning as described herein. For example, these cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 35 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to or following administration of the composition of the described herein.

### Selection of Donor Cells

In some embodiments, the subject is the donor. In such cases, withdrawn cells (e.g., pluripotent cells, ESCs, iPSCs, multipotent cells, CD34+ cells, HSCs, MPCs, BLPCs, monocytes, macrophages, microglial progenitor cells, or microglia) may be re-infused into the subject (following modification (e.g., incorporation of the transgene encoding TREM2, and/or disruption of endogenous TREM2), such that the cells may subsequently home to hematopoietic tissue and establish productive hematopoiesis, thereby populating or repopulating a line of cells that is defective or deficient in the subject (e.g., a population of microglia). In this scenario, the transplanted cells are least likely to undergo graft rejection, as the infused cells are derived from the subject and express the same HLA class me and class II antigens as expressed by the subject. Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

The number of cells administered to a subject for the treatment of an NCD (e.g., AD, PLOSL, FTLD, or PD (e.g., TREM2-associated AD, PLOSL, FTLD, or PD)) as described herein may depend, for example, on the expression level of TREM2, the subject, pharmaceutical formulation methods, administration methods (e.g., administration time and administration route), the subject's age, body weight, sex, severity of the disease being treated, and whether or not the subject has been treated with agents to ablate endogenous microglia. The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of cells may be administered in any suitable dosage following conditioning. Non-limiting examples of dosages are about 1×10⁵ as cells/kg of recipient to about 1×10⁷ cells/kg (e.g., from about 2×10⁵ as cells/kg to about 9×10⁶ cells/kg, from about 3×10⁵ as cells/kg to about 8×10⁶ cells/kg, from about 4×10⁵ as cells/kg to about 7×10⁶ cells/kg, from about 5×10⁵ as cells/kg to about 6×10⁶ cells/kg, from about 5×10⁵ as cells/kg to about 1×10⁷ cells/kg, from about 6×10⁵ as cells/kg to about 1×10⁷ cells/kg, from about 7×10⁵ as cells/kg to about 1×10⁷ cells/kg, from about 8×10⁵ as cells/kg to about 1×10⁷ cells/kg, from about 9×10⁵ as cells/kg to about 1×10⁷ cells/kg, and from about 1×10⁶ cells/kg to about 1×10⁷ cells/kg). Additional exemplary dosages are from about 1×10¹⁰ cells/kg of recipient to about 1×10¹² cells/kg (e.g., from about 2×10¹⁰ cells/kg to about 9×10¹¹ cells/kg, from about 3×10¹⁰ cells/kg to about 8×10¹¹ cells/kg, from about 4×10¹⁰ cells/kg to about 7×10¹¹ cells/kg, from about 5×10¹⁰ cells/kg to about 6×10¹¹ cells/kg, from about 5×10¹⁰ cells/kg to about 1×10¹² cells/kg, from about 6×10¹⁰ cells/kg to about 1×10¹² cells/kg, from about 7×10¹⁰ cells/kg to about 1×10¹² cells/kg, from about 8×10¹⁰ cells/kg to about 1×10¹² cells/kg, from about 9×10¹⁰ cells/kg to about 1×10¹² cells/kg, and from about 1×10¹¹ cells/kg to about 1×10¹² cells/kg), among others.

The cells and compositions described herein can be administered in an amount sufficient to improve one or more pathological features in the NCD. Administration of the cells or compositions described herein may increase the quantity of microglia expressing an anti-inflammatory phenotype in the brain of the subject relative to the quantity of microglia expressing a pro-inflammatory phenotype in the brain of the subject, decrease the level of pro-inflammatory cytokines in the brain of the subject, increase the level of anti-inflammatory cytokines in the brain of the subject, improve the cognitive performance of the subject, improve the motor function of the subject, reduce amyloid-3 and neurofibrillary tau protein levels or aggregation thereof in the subject, reduce demyelination, reduce the quantity or size of axonal spheroids, reduce occurrence or severity of epileptic seizures, reduce pain in distal extremities (e.g., ankles, feet, wrists, or hands), reduce osseous cysts, reduce bone fractures, reduce motor impairments, reduce vascular pathology, reduce the accumulation of lipid-laden macrophages or free fatty acids in the brain, and/or reduce loss of brain tissue in the subject. The numbers of microglia with pro-inflammatory or anti-inflammatory phenotypes may be assessed using ELISAs to compare the level of cytokines, chemokines, and other pro- and anti-inflammatory mediators in the cerebrospinal fluid (CSF) of subjects before and after treatment, by using PET imaging to view translocator protein (TSPO), a protein highly expressed in microglia expressing a pro-inflammatory signature, before and after treatment, e.g., using TSPO radioligand ¹¹C-(R)PK11195, or by analyzing the levels of inflammatory- and anti-inflammatory genes and proteins in a tissue sample using standard techniques, e.g., western blot analysis, immunohistochemical analyses, or quantitative RT-PCR. Cognition and motor function can be assessed using standard neurological tests before and after treatment and amyloid-3 and tau proteins can be detected in plasma and CSF using ELISA. Neurodegeneration can be assessed using F18-fluorodeoxyglucose PET scans or MRI scans. The subject may be evaluated 1 month, 2 months, 3 months, 4 months, 5 months, 6 months or more following administration of the population of cells depending on the route of administration used for treatment. Depending on the outcome of the evaluation, the subject may receive additional treatments.

### Kits

The compositions described herein can be provided in a kit for use in treating an NCD (e.g., AD, PLOSL, FTLD, or PD). Compositions may include host cells described herein (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding TREM2 (e.g., a transgene capable of expression in macrophages or microglia), and, optionally, may have disrupted endogenous TREM2. Cells may be cryopreserved, e.g., in dimethyl sulfoxide (DMSO), glycerol, or another cryoprotectant. The kit can include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation of a Cell Containing a Transgene Encoding Triggering Receptor Expressed on Myeloid Cells Two

An exemplary method for making cells (e.g., pluripotent cells, ESCs, iPSCs, multipotent cells, CD34+ cells, HSCs, MPCs, BLPCs, monocytes, macrophages, microglial progenitor cells, or microglia) that contain a transgene encoding triggering receptor expressed on myeloid cells two (TREM2) for use in the compositions and methods described herein is by way of transduction. Retroviral vectors (e.g., a lentiviral vector, alpharetroviral vector, or gammaretroviral vector) containing a high expression constitutive promoter, such as the MND promoter (SEQ ID NO. 14 or SEQ ID NO. 16) or the CD11b promoter (SEQ ID NO. 17), and the polynucleotide encoding TREM2 can be engineered using standard techniques known in the art. After the retroviral vector is engineered, the retrovirus can be used to transduce cells to generate a population of cells that express TREM2.

Additional exemplary methods for making cells that contain a transgene encoding TREM2 for use in the compositions and methods described herein is transfection. Using molecular biology techniques known in the art, plasmid DNA containing a promoter, such as a high expression constitutive promoter, (e.g., the MND promoter or CD11b promoter), and the polynucleotide encoding TREM2 can be produced. For example, the TREM2 gene may be amplified from a human cell line using PCR-based techniques known in the art, or the gene may be synthesized, for example, using solid-phase polynucleotide synthesis procedures. The TREM2 gene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that express TREM2. In both exemplary methods described herein, the TREM2 may be expressed as a TREM2 fusion protein. The TREM2 fusion protein may contain a peptide sequence containing the LDLRf Rb domain of ApoE to allow for the penetrance of the TREM2 fusion protein across the blood-brain barrier.

### Example 2. Administration of a Population of Containing a Transgene Encoding TREM2 to a Subject Suffering from a Neurocognitive Disease

According to the methods disclosed herein, a physician of skill in the art can treat a subject, such as a human subject, so as to reduce or alleviate symptoms of an NCD, e.g., Alzheimer's disease (AD), Nasu-Hakola disease (PLOSL), frontotemporal lobar degeneration (FTLD), or Parkinson disease (PD). To this end, a physician of skill in the art can administer to the human subject a population of cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding TREM2 (e.g., a transgene capable of expression in macrophages or microglia). The cells can be transduced or transfected ex vivo to express TREM2 using techniques described herein or known in the art. The population of cells containing the transgene encoding TREM2 operably linked to an MND promoter (SEQ ID NO. 14 or SEQ ID NO. 16) or CD11b promoter (SEQ ID NO. 17) may be administered to the subject, for example, systemically (e.g., intravenously), directly to the CNS (e.g., intracerebroventricularly or stereotactically), or directly into the bone marrow (e.g., intraosseously), to treat an NCD. The cells can also be administered to the subject by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the subject, one or more agents may be administered to the subject to ablate the subject's endogenous microglia and/or hematopoietic stem and progenitor cells, for example, busulfan, treosulfan, PLX3397, PLX647, PLX5622, and/or clodronate liposomes. Other methods of cell ablation well known in the art, such as irradiation, may be used alone or in combination with one or more of the aforementioned agents to ablate the subject's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the subject after microglial ablation, the cells can repopulate the brain, differentiating into microglia. The population of cells can be administered to the subject from, for example, 1 week to 1 month (e.g., 1 week, 2 weeks, 3 weeks, 4, weeks) or more after microglial ablation.

Following ablation of the subject's endogenous microglia and/or hematopoietic stem and progenitor cells, a population of cells may be administered to the subject systemically (e.g., intravenously), or by bone marrow transplantation to reconstitute the bone marrow compartment. The number of cells may be administered in any suitable dosage following conditioning. Non-limiting examples of dosages are about 1×10⁵ as cells/kg of recipient to about 1×10⁷ cells/kg (e.g., from about 2×10⁵ as cells/kg to about 9×10⁶ cells/kg, from about 3×10⁵ as cells/kg to about 8×10⁶ cells/kg, from about 4×10⁵ as cells/kg to about 7×10⁶ cells/kg, from about 5×10⁵ as cells/kg to about 6×10⁶ cells/kg, from about 5×10⁵ as cells/kg to about 1×10⁷ cells/kg, from about 6×10⁵ as cells/kg to about 1×10⁷ cells/kg, from about 7×10⁵ as cells/kg to about 1×10⁷ cells/kg, from about 8×10⁵ as cells/kg to about 1×10⁷ cells/kg, from about 9×10⁵ as cells/kg to about 1×10⁷ cells/kg, or from about 1×10⁶ cells/kg to about 1×10⁷ cells/kg, among others). Administration may occur prior to or following administration of the cells containing a transgene encoding TREM2. The population of cells can be administered to the subject in an amount sufficient to treat one or more of the pathological features of an NCD. For example, the population of cells can be administered in an amount sufficient to increase the quantity of microglia exhibiting an anti-inflammatory phenotype in the brain of the subject relative to the quantity of microglia exhibiting a pro-inflammatory phenotype in the brain of the subject. The relative increase can be measured using conventional techniques known in the art, such as by performing an ELISA on subject CSF before and after treatment to assess the level of pro-inflammatory and anti-inflammatory cytokines secreted by microglia at both time points. A standard neurological examination can also be performed by the physician before and after treatment to evaluate changes in cognitive performance and motor function. The subject may be evaluated, for example, 1 month, 2 months, 3 months, 4 months, 5 months, 6 months or more following administration of the population of cells depending on the route of administration used for treatment. A finding of reduced pro-inflammatory cytokines, increased anti-inflammatory cytokines, reduction in amyloid-3 and/or neurofibrillary tau protein levels or aggregation thereof, reduced epileptic seizure occurrence or severity, reduced pain in the distal extremities (e.g., ankles, feet, wrists, or hands), reduced occurrence of bone fractures, and/or improved cognitive or motor function following administration of a population of cells containing a transgene encoding TREM2 provides an indication that the treatment has successfully treated the NCD.

### Example 3. Disruption of Endogenous TREM2 in Cells Prior to Administration to a Subject Suffering from a Neurocognitive Disorder

In any of the methods disclosed herein, the cells (e.g., pluripotent cells, ESCs, iPSCs, multipotent cells, CD34+ cells, HSCs, MPCs, BLPCs, monocytes, macrophages, microglial progenitor cells, or microglia) may be treated to disrupt the endogenous TREM2 prior to administration to the subject. (e.g., a subject diagnosed with an NCD, such as, e.g., AD, PLOSL, FTLD, or PD). An exemplary method of disrupting endogenous TREM2 in cells is using a CRISPR/Cas system (e.g., CRISPR/Cas9 or CRISPR/Cas12a) with a TREM2-specific guide RNA (gRNA) to induce one or more double-strand breaks (DSB). Following non-homologous end joining (NHEJ) to repair the DSB, the presence of newly-formed indel mutations will result in endogenous TREM2 disruption. Alternative methods for disruption of endogenous TREM2 by site-specifically cleaving genomic DNA prior to the incorporation of a TREM2 transgene in a cell include the use of zinc finger nucleases (ZFNs) and transcription activator-like effector nucleases (TALENs). Unlike the CRISPR/Cas system, these enzymes do not contain a guiding polynucleotide to localize to a specific target sequence, but instead rely on internal DNA biding domains within the enzymes to mediate target specificity. In exemplary embodiments, the cell is manipulated ex vivo by the nuclease to decrease or reduce the expression of endogenous TREM2 by 5% or more (e.g., 5%, 10%, 20%, 30%, 40%, 50%, 60%, 70%, 80%, 90%, 95% or more).

### Example 4. Generation of Mammalian Cell Lines Expressing TREM2

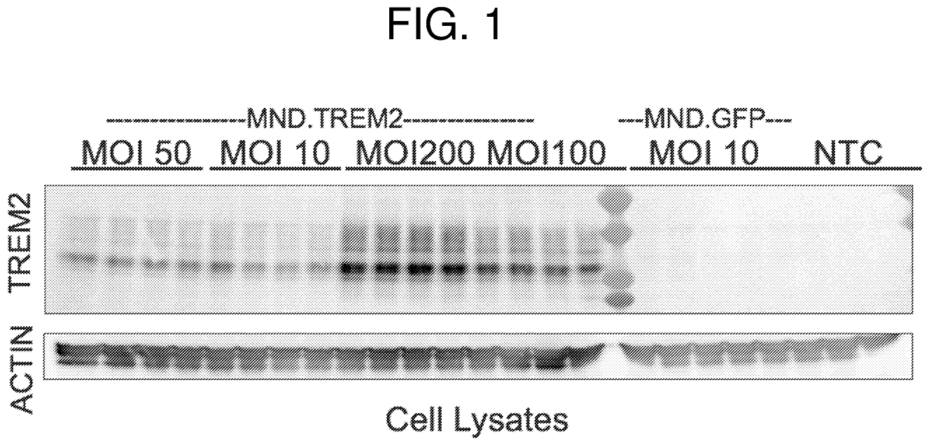

To assess the ability of lentivirally-encoded, codon-optimized TREM2 transgenes to stably express in mammalian cell lines, murine RAW macrophage cell lines, murine primary microglia, and murine lineage negative (Lin−) negative cells were transduced in vitro. In a first experiment, murine RAW macrophage cells were either transduced with a lentiviral vector carrying a transgene encoding the human TREM2 protein (MND.TREM2) or GFP (MND.GFP) at a multiplicity of infection (MOI) of 10, 50, 100, or 200. A separate set of control cells were not transduced (NTC). TREM2 expression was assessed using an antibody raised against human TREM2. Stable expression of human TREM2 was observed in murine macrophages (FIG. 1).

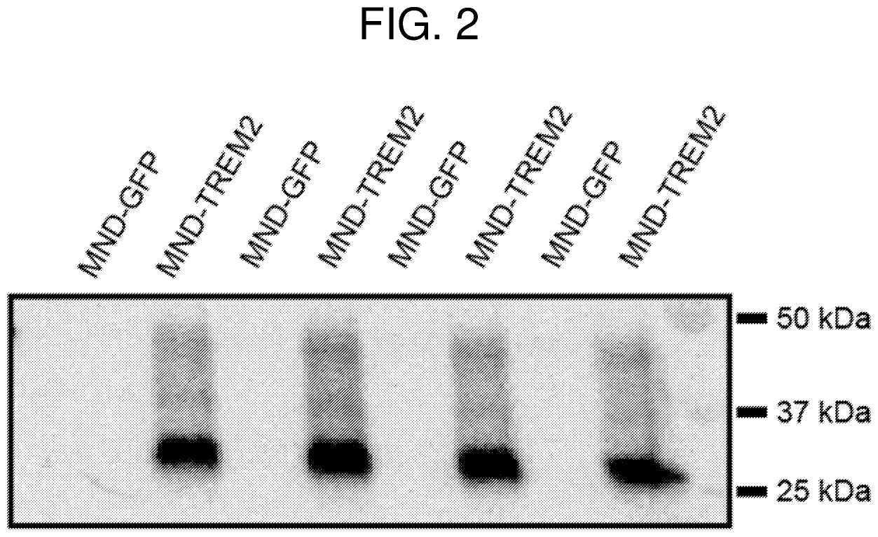

In a separate experiment, murine primary microglia were either transduced with a lentiviral vector carrying a transgene encoding the human TREM2 protein (MND-TREM2) or GFP (MND-GFP). A separate set of control cells were not transduced (NT). TREM2 expression was assessed using an antibody raised against human TREM2. Stable expression of human TREM2 was observed in murine primary microglia (FIG. 2).

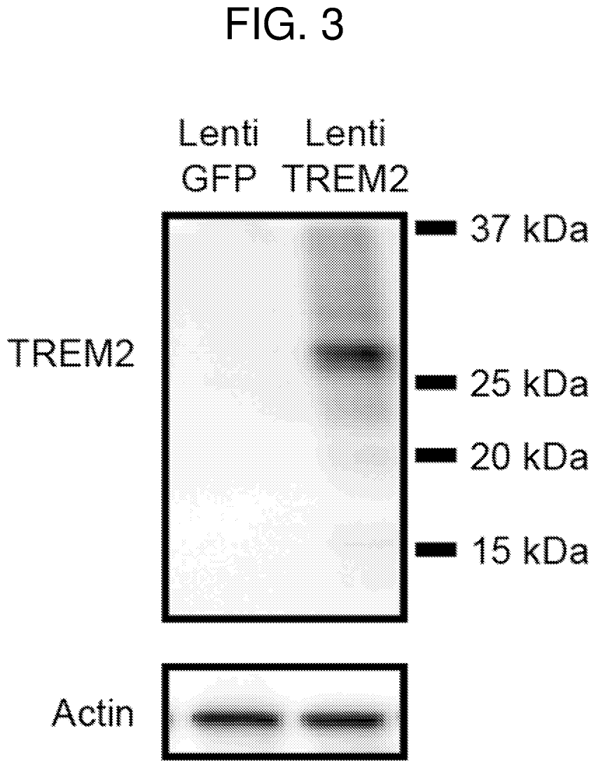

In another experiment, murine Lin− cells were either transduced with a lentiviral vector carrying a transgene encoding the human TREM2 protein (Lenti TREM2) or GFP (Lenti GFP). TREM2 expression was assessed using an antibody raised against human TREM2. Stable expression of human TREM2 was observed in murine Lin− cells. (FIG. 3).

Combined, the above results demonstrate that stable expression of codon-optimized human TREM2 protein can be achieved in vitro using lentiviral vectors, resulting in increased levels of TREM2 in immortalized murine macrophages, primary microglia, and Lin− cells in which human TREM2 is normally absent. These findings demonstrate a potential therapeutic approach for diseases caused by or associated with mutations in the TREM2 gene.

### Example 5: Macrophage-Specific Expression of a Heterologous Transgene Expressing TREM2 in Mice In Vivo

To determine the blood cell-specific tropism of plasmid vectors encoding TREM2 under regulatory control of an MND promoter (SEQ ID NO. 14)(“MND.TREM2”), MND.TREM2 vectors or vectors encoding GFP linked to the MND promoter (“MND.GFP”) were administered to mice. Lineage-negative cells were isolated from donor mice and transduced with either an MND.GFP or MND.TREM2 vector in vitro. Recipient animals were administered 0.5×10⁶ MND.GFP- or MND.TREM2-transduced lineage-negative cells via intravenous injection. Thirty days after treatment, peripheral blood was drawn from mice treated with MND.TREM2, MND.GFP, and untreated mice. TREM2 expression was assessed using an antibody raised against human TREM2. GFP expression was assessed via fluorescence of the transgene using flow cytometry.

Heterologous TREM2 transgene expression was observed almost exclusively in cells of the myeloid lineage (FIG. 4A), with near-zero expression measured in T-cells and B-cells. Unlike TREM2, MND-driven expression of GFP was observed at high levels in myeloid cells, T-cells, and B-cells (FIG. 4B). The levels of TREM2 protein expression (FIG. 4C) and vector copy number (VCN; FIG. 4D) in peripheral blood were also quantified according to the following protocol. TREM2 protein levels were measured using a commercial ELISA kit from R&D systems on lysed whole blood samples collected from MND.TREM2-treated animals 60 days post-dose. VCN was used to quantify the number of integrated vectors per mouse genome from the same sample using quantitative polymerase chain reaction (qPCR) with primers specific to the WPRE element of the MND.TREM2 vector and a housekeeping gene in the mouse genome.

Therefore, the MND.TREM2 vector is capable of driving myeloid-cell specific expression particularly in cells of the myeloid lineage. These findings suggest that TREM2 cell-surface localization is limited to its physiologically-relevant cell type (i.e., myeloid cells, e.g., monocytes that can give rise to tissue-resident macrophages, such as microglia-like cells). Furthermore, these findings indicate a potential therapeutic avenue for targeting TREM2 expression to cells of the myeloid lineage using vectors encoding TREM2 via high expression constitutive promoters, such as, e.g., the MND promoter.

### Example 6: Comparison of Cell-Type Specific Differences in Promoter-Driven Expression of a Transgene Across Cells of the Myeloid and Lymphoid Lineages

To determine the relative strength of different promoters in controlling cell-type specific expression of a heterologous transgene in cells of the myeloid and lymphoid lineages, several promoters, including a phosphoglycerate kinase (PGK) promoter, embryonal fyn-associated substrate (EFS) promoter, MND (SEQ ID NO. 14) promoter, and integrin subunit alpha M (CD11b) promoter (SEQ ID NO: 17), were operably linked to a transgene encoding GFP and incorporated into lentiviral expression plasmids that were used to generate viruses. The viruses were then transduced into mouse hematopoietic stem cells, which were used for transplant into wild type C57/B16 mice after busulfan conditioning. GFP expression was quantified via flow cytometry.

GFP expression within the myeloid compartment (e.g., monocytes, granulocytes, and microglia) of the bone marrow (FIG. 5A), blood (FIG. 5B), and brain (FIG. 5C) was enhanced using the MND and CD11b promoters as compared to commonly used PGK and EFS promoters. These findings indicate that cell type-specific expression can be fine-tuned in specific cell lineages by employing the high expression constitutive expression promoters (e.g., MND and CD11b promoters) described herein, thereby creating an opportunity to enhance the therapeutic efficacy and safety of vector-encoded therapeutic agents and cells containing the same described herein.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

What is claimed is:

1. A method of treating a subject diagnosed as having a neurocognitive disorder (NCD) the method including administering to the subject a composition comprising a population of cells containing a transgene encoding one or more triggering receptor expressed on myeloid cells two (TREM2) proteins operably linked to a high expression constitutive promoter.

2. The method of claim 1, wherein the high expression constitutive promoter is a Myeloproliferative Sarcoma Virus Enhancer, Negative Control Region Deleted, dl587rev Primer Binding Site Substituted (MND) promoter.

3. The method of claim 2, wherein the MND promoter comprises a polynucleotide having at least 85% sequence identity to the nucleic acid sequence of SEQ ID NO. 14 or SEQ ID NO. 16, optionally wherein the MND promoter comprises a polynucleotide having at least 90% sequence identity to the nucleic acid sequence of SEQ ID NO. 14 or SEQ ID NO. 16, optionally wherein the MND promoter comprises a polynucleotide having at least 95% sequence identity to the nucleic acid sequence of SEQ ID NO. 14 or SEQ ID NO. 16, optionally wherein the MND promoter comprises a polynucleotide having the nucleic acid sequence of SEQ ID NO. 14 or SEQ ID NO. 16.

4. The method of claim 1, wherein the high expression constitutive promoter is an integrin subunit alpha M (CD11b) promoter.

5. The method of claim 4, wherein the CD11b promoter comprises a polynucleotide having at least 85% sequence identity to the nucleic acid sequence of SEQ ID NO. 17, optionally wherein the CD11b promoter comprises a polynucleotide having at least 90% sequence identity to the nucleic acid sequence of SEQ ID NO. 17, optionally wherein the CD11b promoter comprises a polynucleotide having at least 95% sequence identity to the nucleic acid sequence of SEQ ID NO. 17, optionally wherein the CD11b promoter comprises a polynucleotide having the nucleic acid sequence of SEQ ID NO. 17.

6. The method of any one of claims 1-5, wherein the NCD is a major NCD.

7. The method of claim 6, wherein the major NCD interferes with the subject's independence and/or normal daily functioning.

8. The method of claim 6 or 7, wherein the major NCD is associated with a score obtained by the subject on a cognitive test that is at least two standard deviations away from the mean score of a reference population.

9. The method of any one of claims 1-5, wherein the NCD is a mild NCD.

10. The method of claim 9, wherein the mild NCD does not interfere with the subject's independence and/or normal daily functioning.

11. The method of claim 9 or 10, wherein the mild NCD is associated with a score obtained by the subject on a cognitive test that is between one to two standard deviations away from the mean score of a reference population.

12. The method of claim 8 or 11, wherein the reference population is a general population.

13. The method of claim 8, 11, or 12, wherein the cognitive test is selected from the group consisting of Eight item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE).

14. The method of any one of claims 1-13, wherein the NCD is associated with impairment in one or more of complex attention, executive function, learning and memory, language, perceptual motor function, and social cognition.

15. The method of any one of claims 1-14, wherein the NCD is not due to delirium or other mental disorder.

16. The method of any one of claims 1-15, wherein the NCD is Alzheimer's disease (AD).

17. The method of any one of claims 1-15, wherein the NCD is a leukodystrophy.

18. The method of claim 17, wherein the leukodystrophy is Nasu-Hakola disease (PLOSL).

19. The method of any one of claims 1-18, wherein the transgene encodes a TREM2 protein having an amino acid sequence that is at least 85% identical to the amino acid sequence of SEQ ID NO. 1, optionally wherein the TREM2 protein has an amino acid sequence that is at least 90% identical to the amino acid sequence of SEQ ID NO. 1, optionally wherein the TREM2 protein has an amino acid sequence that is at least 95% identical to the amino acid sequence of SEQ ID NO. 1, optionally wherein the TREM2 protein has an amino acid sequence of SEQ ID NO. 1.

20. The method of any one of claims 1-18, wherein the transgene encodes a TREM2 protein having an amino acid sequence that is at least 85% identical to the amino acid sequence of SEQ ID NO. 2, optionally wherein the TREM2 protein has an amino acid sequence that is at least 90% identical to the amino acid sequence of SEQ ID NO. 2, optionally wherein the TREM2 protein has an amino acid sequence that is at least 95% identical to the amino acid sequence of SEQ ID NO. 2, optionally wherein the TREM2 protein has an amino acid sequence of SEQ ID NO. 2.

21. The method of any one of claims 1-18, wherein the transgene encodes a TREM2 protein having an amino acid sequence that is at least 85% identical to the amino acid sequence of SEQ ID NO. 3, optionally wherein the TREM2 protein has an amino acid sequence that is at least 90% identical to the amino acid sequence of SEQ ID NO. 3, optionally wherein the TREM2 protein has an amino acid sequence that is at least 95% identical to the amino acid sequence of SEQ ID NO. 3, optionally wherein the TREM2 protein has an amino acid sequence of SEQ ID NO. 3.

22. The method of any one of claims 1-21, wherein the TREM2 is a full-length TREM2.

23. The method of any one of claims 1-22, wherein the TREM2 comprises a TREM2 signal peptide.

24. The method of any one of claims 1-21, wherein the TREM2 is a soluble TREM2 (sTREM2), a TREM2 C-terminal fragment (TREM2-CTF), a TREM2 intracellular domain (TREM2-ICD), a TREM2 A-β-like (TREM2-T23) peptide.

25. The method of any one of claims 1-24, wherein the TREM2 lacks a functional ectodomain cleavage site or a functional intramembrane cleavage site.

26. The method of any one of claims 1-25, wherein the transgene encodes two or more TREM2 proteins.

27. The method of any one of claims 1-26, wherein the transgene comprises a polynucleotide having at least 85% sequence identity to the nucleic acid sequence of SEQ ID NO. 4, optionally wherein the transgene comprises a polynucleotide having at least 90% sequence identity to the nucleic acid sequence of SEQ ID NO. 4, optionally wherein the transgene comprises a polynucleotide having at least 95% sequence identity to the nucleic acid sequence of SEQ ID NO. 4, optionally wherein the Transgene comprises a polynucleotide having the nucleic acid sequence of SEQ ID NO. 4.

28. The method of any one of claims 1-27, wherein the transgene comprises a polynucleotide having at least 85% sequence identity to the nucleic acid sequence of SEQ ID NO. 5, optionally wherein the transgene comprises a polynucleotide having at least 90% sequence identity to the nucleic acid sequence of SEQ ID NO. 5, optionally wherein the transgene comprises a polynucleotide having at least 95% sequence identity to the nucleic acid sequence of SEQ ID NO. 5, optionally wherein the transgene comprises a polynucleotide having the nucleic acid sequence of SEQ ID NO. 5.

29. The method of any one of claims 1-28, wherein the transgene comprises a polynucleotide having at least 85% sequence identity to the nucleic acid sequence of SEQ ID NO. 6, optionally wherein the transgene comprises a polynucleotide having at least 90% sequence identity to the nucleic acid sequence of SEQ ID NO. 6, optionally wherein the transgene comprises a polynucleotide having at least 95% sequence identity to the nucleic acid sequence of SEQ ID NO. 6, optionally wherein the transgene comprises a polynucleotide having the nucleic acid sequence of SEQ ID NO. 6.

30. The method of any one of claims 1-29, wherein the transgene comprises a polynucleotide having at least 85% sequence identity to the nucleic acid sequence of SEQ ID NO. 7, optionally wherein the transgene comprises a polynucleotide having at least 90% sequence identity to the nucleic acid sequence of SEQ ID NO. 7, optionally wherein the transgene comprises a polynucleotide having at least 95% sequence identity to the nucleic acid sequence of SEQ ID NO. 7, optionally wherein the transgene comprises a polynucleotide having the nucleic acid sequence of SEQ ID NO. 7.

31. The method of any one of claims 1-30, wherein the transgene comprises a polynucleotide having at least 85% sequence identity to the nucleic acid sequence of SEQ ID NO. 9, optionally wherein the transgene comprises a polynucleotide having at least 90% sequence identity to the nucleic acid sequence of SEQ ID NO. 9, optionally wherein the transgene comprises a polynucleotide having at least 95% sequence identity to the nucleic acid sequence of SEQ ID NO. 9, optionally wherein the transgene comprises a polynucleotide having the nucleic acid sequence of SEQ ID NO. 9.

32. The method of any one of claims 1-31, wherein the transgene comprises a polynucleotide having at least 85% sequence identity to the nucleic acid sequence of SEQ ID NO. 11, optionally wherein the transgene comprises a polynucleotide having at least 90% sequence identity to the nucleic acid sequence of SEQ ID NO. 11, optionally wherein the transgene comprises a polynucleotide having at least 95% sequence identity to the nucleic acid sequence of SEQ ID NO. 11, optionally wherein the transgene comprises a polynucleotide having the nucleic acid sequence of SEQ ID NO. 11.

33. The method of any one of claims 1-32, wherein the transgene is a codon optimized TREM2 transgene having at least 85% sequence identity to the nucleic acid sequence of any one of SEQ ID NOs. 8, 10, or 12, optionally wherein the codon optimized TREM2 transgene comprises a polynucleotide having at least 90% sequence identity to the nucleic acid sequence of any one of SEQ ID NOs. 8, 10, or 12, optionally wherein the codon optimized TREM2 transgene comprises a polynucleotide having at least 95% sequence identity to the nucleic acid sequence of any one of SEQ ID NOs. 8, 10, or 12, optionally wherein the codon optimized TREM2 transgene comprises a polynucleotide having the nucleic acid sequence of any one of SEQ ID NOs. 8, 10, or 12.

34. The method of any one of claims 1-33, wherein the TREM2 is a TREM2 fusion protein.

35. The method of claim 34, wherein the TREM2 fusion protein comprises a receptor-binding (Rb) domain of apolipoprotein E (ApoE).

36. The method of claim 35, wherein the Rb domain comprises a portion of ApoE having the amino acid sequence of residues 25-185, 50-180, 75-175, 100-170, 125-160, or 130-150 of SEQ ID NO. 13.

37. The method of claim 35 or 36, wherein the Rb domain comprises a region having at least 70% sequence identity to the amino acid sequence of residues 159-167 of SEQ ID NO. 13.

38. The method of any one of claims 1-37, wherein the transgene encoding TREM2 further comprises a microRNA (miRNA)-126 (miR-126) targeting sequence in the 3′ UTR.

39. The method of any one of claims 16-38, wherein the AD or PLOSL is TREM2-associated AD or PLOSL.

40. The method of any one of claims 1-39, wherein the cells are pluripotent cells or multipotent cells.

41. The method of claim 40, wherein the multipotent cells are CD34+ cells.

42. The method of claim 41, wherein the CD34+ cells are hematopoietic stem cells (HSCs) or myeloid cells.

43. The method of claim 42, wherein the myeloid cells are myeloid progenitor cells (MPCs).

44. The method of claim 37, wherein the pluripotent cells are embryonic stem cells (ESCs) or induced pluripotent stem cells (iPSCs).

45. The method of any one of claims 1-40, wherein the cells are blood lineage progenitor cells (BLPCs), microglial progenitor cells, monocytes, macrophages, or microglia.

46. The method of claim 45, wherein the BLPCs are monocytes.

47. The method of any one of claims 1-46, wherein, upon administration of the cells to the subject, expression of the TREM2 transgene is higher in myeloid cells in the subject than in lymphoid cells in the subject.

48. The method of claim 47, wherein, upon administration of the cells to the subject, expression of the TREM2 transgene is higher in myeloid cells in the subject by at least 50% than in lymphoid cells in the subject; optionally wherein expression of the TREM2 transgene is higher in myeloid cells in the subject by at least 60% than in lymphoid cells in the subject; optionally wherein expression of the TREM2 transgene is higher in myeloid cells in the subject by at least 70% than in lymphoid cells in the subject; optionally wherein expression of the TREM2 transgene is higher in myeloid cells in the subject by at least 80% than in lymphoid cells in the subject; optionally wherein expression of the TREM2 transgene is higher in myeloid cells in the subject by at least 90% than in lymphoid cells in the subject; optionally wherein expression of the TREM2 transgene is higher in myeloid cells in the subject by at least 95% than in lymphoid cells in the subject; optionally wherein expression of the TREM2 transgene is higher in myeloid cells in the subject by at least 100% than in lymphoid cells; optionally wherein expression of the TREM2 transgene is higher in myeloid cells in the subject by at least 150% than in lymphoid cells; optionally wherein expression of the TREM2 transgene is higher in myeloid cells in the subject by at least 200% than in lymphoid cells; optionally wherein expression of the TREM2 transgene is higher in myeloid cells in the subject by at least 250% than in lymphoid cells; optionally wherein expression of the TREM2 transgene is higher in myeloid cells in the subject by at least 300% than in lymphoid cells; optionally wherein expression of the TREM2 transgene is higher in myeloid cells in the subject by at least 350% than in lymphoid cells; optionally wherein expression of the TREM2 transgene is higher in myeloid cells in the subject by at least 400% than in lymphoid cells; optionally wherein expression of the TREM2 transgene is higher in myeloid cells in the subject by at least 450% than in lymphoid cells; optionally wherein expression of the TREM2 transgene is higher in myeloid cells in the subject by at least 500% than in lymphoid cells.

49. The method of claim 47 or 48, wherein the lymphoid cells are B cells or T cells.

50. The method of any one of claims 47-49, wherein the expression level of the TREM2 transgene is measured in one or more organs, tissues, or body fluids of the subject.

51. The method of claim 50, wherein the one or more body fluids is peripheral blood.

52. The method of any one of claims 1-51, wherein a population of endogenous microglia in the subject has been ablated prior to administration of the composition.

53. The method of any one of claims 1-51, the method comprising ablating a population of endogenous microglia in the subject prior to administering the composition to the subject.

54. The method of claim 52 or 53 wherein the endogenous microglia are ablated using an agent selected from the group consisting of busulfan, PLX3397, PLX647, PLX5622, treosulfan, and clodronate liposomes, by radiation therapy, or a combination thereof.

55. The method of any one of claims 1-54, wherein the composition is administered to the subject by way of systemic administration, by way of direct administration to the central nervous system of the subject, by way of direct administration to the bone marrow of the subject, or by way of bone marrow transplant comprising the composition.

56. The method of any one of claims 1-55, the method further comprising administering to the subject a population of cells.

57. The method of claim 56, wherein the population of cells is administered to the subject prior to administration of the composition or following administration of the composition.

58. The method of claim 56 or 57, wherein the cells are pluripotent cells or multipotent cells.

59. The method of claim 58, wherein the multipotent cells are CD34+ cells.

60. The method of claim 59, wherein the CD34+ cells are HSCs or myeloid cells.

61. The method of claim 60, wherein the myeloid cells are MPCs.

62. The method of claim 58, wherein the pluripotent cells are ESCs or iPSCs.

63. The method of any one of claims 56 or 57, wherein the cells are BLPCs, microglial progenitor cells, monocytes, macrophages, or microglia.

64. The method of claim 63, wherein the BLPCs are monocytes.

65. The method of any one of claims 56-64, wherein the cells are not modified to express a transgene encoding TREM2.

66. The method of any one of claims 1-65, wherein, prior to administration of the composition to the subject, endogenous TREM2 is disrupted in the cells, subject, or a population of neurons in the subject.

67. The method of claim 66, wherein the endogenous TREM2 is disrupted by contacting the cells with a nuclease that catalyzes cleavage of an endogenous TREM2 nucleic acid in the cells.

68. The method of claim 67, wherein the nuclease is a CRISPR-associated protein 9 (Cas9), CRISPR-associated protein 12a (Cas12a), a transcription activator like effector nuclease, a meganuclease, or a zinc finger nuclease.

69. The method of any one of claims 66-68, wherein the endogenous TREM2 is disrupted by administering an inhibitory RNA molecule to the cells, the subject, or the population of neurons.

70. The method of claim 69, wherein the inhibitory RNA molecule is a short interfering RNA, a short hairpin RNA, or a miRNA.

71. The method of any one of claims 1-70, wherein the cells are autologous cells or allogeneic cells.

72. The method of any one of claims 1-71, wherein the cells are transfected or transduced ex vivo to express the TREM2.

73. The method of claim 72, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

74. The method of claim 73, wherein the viral vector is a Retroviridae family viral vector.

75. The method of claim 74, wherein the Retroviridae family viral vector is a lentiviral vector, alpharetroviral vector, or gamma retroviral vector.

76. The method of any one of claims 73-75, wherein the Retroviridae family viral vector comprises a central polypurine tract, a woodchuck hepatitis virus post transcriptional regulatory element, a 5′ LTR, HIV signal sequence, HIV Psi signal 5′ splice site, delta GAG element, 3′ splice site, and a 3′ self inactivating LTR.

77. The method of claim 73, wherein the viral vector is an AAV selected from the group consisting of AAV1, AAV2, AAV3, AAV4, AAV5, AAV6, AAV7, AAV8, AAV9, AAV10, and AAVrh74.

78. The method of any one of claims 73-77, wherein the viral vector is a pseudotyped viral vector.

79. The method of claim 78, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

80. The method of any one of claims 73-79, wherein the viral vector comprises a polynucleotide having at least 85% sequence identity to the nucleic acid sequence of SEQ ID NO. 15, optionally wherein the viral vector comprises a polynucleotide having at least 90% sequence identity to the nucleic acid sequence of SEQ ID NO. 15, optionally wherein the viral vector comprises a polynucleotide having at least 95% sequence identity to the nucleic acid sequence of SEQ ID NO. 15, optionally wherein the viral vector comprises a polynucleotide having the nucleic acid sequence of SEQ ID NO. 15.

81. The method of any one of claims 73-79, wherein the viral vector comprises a polynucleotide having at least 85% sequence identity to the nucleic acid sequence of SEQ ID NO. 18, optionally wherein the viral vector comprises a polynucleotide having at least 90% sequence identity to the nucleic acid sequence of SEQ ID NO. 18, optionally wherein the viral vector comprises a polynucleotide having at least 95% sequence identity to the nucleic acid sequence of SEQ ID NO. 18, optionally wherein the viral vector comprises a polynucleotide having the nucleic acid sequence of SEQ ID NO. 18.

82. A composition comprising a population of cells that express a transgene encoding TREM2 operably linked to a high expression constitutive promoter.

83. The composition of claim 82, wherein the high expression constitutive promoter is an MND promoter.

84. The composition of claim 83, wherein the MND promoter comprises a polynucleotide having at least 85% sequence identity to the nucleic acid sequence of SEQ ID NO. 14 or SEQ ID NO. 16, optionally wherein the MND promoter comprises a polynucleotide having at least 90% sequence identity to the nucleic acid sequence of SEQ ID NO. 14 or SEQ ID NO. 16, optionally wherein the MND promoter comprises a polynucleotide having at least 95% sequence identity to the nucleic acid sequence of SEQ ID NO. 14 or SEQ ID NO. 16, optionally wherein the MND promoter comprises a polynucleotide having the nucleic acid sequence of SEQ ID NO. 14 or SEQ ID NO. 16.

85. The composition of claim 84, wherein the high expression constitutive promoter is a CD11b promoter.

86. The composition of claim 85, wherein the CD11b promoter comprises a polynucleotide having at least 85% sequence identity to the nucleic acid sequence of SEQ ID NO. 17, optionally wherein the CD11b promoter comprises a polynucleotide having at least 90% sequence identity to the nucleic acid sequence of SEQ ID NO. 17, optionally wherein the CD11b promoter comprises a polynucleotide having at least 95% sequence identity to the nucleic acid sequence of SEQ ID NO. 17, optionally wherein the CD11b promoter comprises a polynucleotide having the nucleic acid sequence of SEQ ID NO. 17.

87. The composition of any one of claims 82-86, wherein the TREM2 is a full-length TREM2.

88. The composition of any one of claims 82-87, wherein the TREM2 or a variant thereof has an amino acid sequence with at least 85% sequence identity to the amino acid sequence of any one of SEQ ID NOS. 1-3.

89. The composition of claim 88, wherein the TREM2 has an amino acid sequence that has at least 85% sequence identity to SEQ ID NO. 1, optionally wherein the TREM2 protein has an amino acid sequence that is at least 90% identical to the amino acid sequence of SEQ ID NO. 1, optionally wherein the TREM2 protein has an amino acid sequence that is at least 95% identical to the amino acid sequence of SEQ ID NO. 1, optionally wherein the TREM2 protein has an amino acid sequence of SEQ ID NO. 1.

90. The composition of claim 88 or 89, wherein the TREM2 has an amino acid sequence that has at least 85% sequence identity to SEQ ID NO. 2, optionally wherein the TREM2 protein has an amino acid sequence that is at least 90% identical to the amino acid sequence of SEQ ID NO. 2, optionally wherein the TREM2 protein has an amino acid sequence that is at least 95% identical to the amino acid sequence of SEQ ID NO. 2, optionally wherein the TREM2 protein has an amino acid sequence of SEQ ID NO. 2.

91. The composition of any one of claims 88-90, wherein the TREM2 has an amino acid sequence that has at least 85% sequence identity of SEQ ID NO. 3, optionally wherein the TREM2 protein has an amino acid sequence that is at least 90% identical to the amino acid sequence of SEQ ID NO. 3, optionally wherein the TREM2 protein has an amino acid sequence that is at least 95% identical to the amino acid sequence of SEQ ID NO. 3, optionally wherein the TREM2 protein has an amino acid sequence of SEQ ID NO. 3.

92. The composition of any one of claims 82-91, wherein the TREM2 comprises a TREM2 signal peptide.

93. The composition of any one of claims 82-92, wherein the TREM2 is a sTREM2, a TREM2-CTF, a TREM2-ICD, or a TREM2-T2P peptide.

94. The composition of any one of claims 82-93, wherein the TREM2 lacks a functional ectodomain cleavage site or a functional intramembrane cleavage site.

95. The composition of any one of claims 82-94, wherein the transgene encodes two or more TREM2 transgenes.

96. The composition of any one of claims 82-95, wherein the transgene comprises a polynucleotide having at least 85% sequence identity to the nucleic acid sequence of SEQ ID NO. 4, optionally wherein the transgene comprises a polynucleotide having at least 90% sequence identity to the nucleic acid sequence of SEQ ID NO. 4, optionally wherein the transgene comprises a polynucleotide having at least 95% sequence identity to the nucleic acid sequence of SEQ ID NO. 4, optionally wherein the transgene comprises a polynucleotide having the nucleic acid sequence of SEQ ID NO. 4.

97. The composition of any one of claims 82-96, wherein the transgene comprises a polynucleotide having at least 85% sequence identity to the nucleic acid sequence of SEQ ID NO. 5, optionally wherein the transgene comprises a polynucleotide having at least 90% sequence identity to the nucleic acid sequence of SEQ ID NO. 5, optionally wherein the transgene comprises a polynucleotide having at least 95% sequence identity to the nucleic acid sequence of SEQ ID NO. 5, optionally wherein the transgene comprises a polynucleotide having the nucleic acid sequence of SEQ ID NO. 5.

98. The composition of any one of claims 82-97, wherein the transgene comprises a polynucleotide having at least 85% sequence identity to the nucleic acid sequence of SEQ ID NO. 6, optionally wherein the transgene comprises a polynucleotide having at least 90% sequence identity to the nucleic acid sequence of SEQ ID NO. 6, optionally wherein the transgene comprises a polynucleotide having at least 95% sequence identity to the nucleic acid sequence of SEQ ID NO. 6, optionally wherein the transgene comprises a polynucleotide having the nucleic acid sequence of SEQ ID NO. 6.

99. The composition of any one of claims 82-98, wherein the transgene comprises a polynucleotide having at least 85% sequence identity to the nucleic acid sequence of SEQ ID NO. 7, optionally wherein the transgene comprises a polynucleotide having at least 90% sequence identity to the nucleic acid sequence of SEQ ID NO. 7, optionally wherein the transgene comprises a polynucleotide having at least 95% sequence identity to the nucleic acid sequence of SEQ ID NO. 7, optionally wherein the transgene comprises a polynucleotide having the nucleic acid sequence of SEQ ID NO. 7.

100. The composition of any one of claims 82-99, wherein the transgene comprises a polynucleotide having at least 85% sequence identity to the nucleic acid sequence of SEQ ID NO. 9, optionally wherein the transgene comprises a polynucleotide having at least 90% sequence identity to the nucleic acid sequence of SEQ ID NO. 9, optionally wherein the transgene comprises a polynucleotide having at least 95% sequence identity to the nucleic acid sequence of SEQ ID NO. 9, optionally wherein the transgene comprises a polynucleotide having the nucleic acid sequence of SEQ ID NO. 9.

101. The composition of any one of claims 82-100, wherein the transgene comprises a polynucleotide having at least 85% sequence identity to the nucleic acid sequence of SEQ ID NO. 11, optionally wherein the transgene comprises a polynucleotide having at least 90% sequence identity to the nucleic acid sequence of SEQ ID NO. 11, optionally wherein the transgene comprises a polynucleotide having at least 95% sequence identity to the nucleic acid sequence of SEQ ID NO. 11, optionally wherein the transgene comprises a polynucleotide having the nucleic acid sequence of SEQ ID NO. 11.

102. The composition of any one of claims 82-101, wherein the transgene is a codon optimized TREM2 transgene.

103. The composition of claim 102, wherein the codon optimized TREM2 transgene comprises a polynucleotide having a nucleic acid sequence having at least 85% sequence identity to the nucleic acid sequence of any one of SEQ ID NOs. 8, 10, or 12, optionally wherein the codon optimized TREM2 transgene comprises a polynucleotide having at least 90% sequence identity to the nucleic acid sequence of any one of SEQ ID NOs. 8, 10, or 12, optionally wherein the codon optimized TREM2 transgene comprises a polynucleotide having at least 95% sequence identity to the nucleic acid sequence of any one of SEQ ID NOs. 8, 10, or 12, optionally wherein the codon optimized TREM2 transgene comprises a polynucleotide having the nucleic acid sequence of any one of SEQ ID NOs. 8, 10, or 12.

104. The composition of any one of claims 82-103, wherein the TREM2 is a TREM2 fusion protein.

105. The composition of claim 104, wherein the TREM2 fusion protein comprises a Rb domain of ApoE.

106. The composition of claim 105, wherein the Rb domain comprises a portion of ApoE having the amino acid sequence of residues 25-185, 50-180, 75-175, 100-170, 125-160, or 130-150 of SEQ ID NO. 13.

107. The composition of claim 105 or 106, wherein the Rb domain comprises a region having at least 70% sequence identity to the amino acid sequence of residues 159-167 of SEQ ID NO. 13.

108. The composition of any one of claims 82-107, wherein the transgene encoding TREM2 further comprises a miR 126 targeting sequence in the 3′ UTR.

109. The composition of any one of claims 82-108, wherein the cells are pluripotent cells or multipotent cells.

110. The composition of claim 109, wherein the multipotent cells are CD34+ cells.

111. The composition of claim 110, wherein the CD34+ cells are HSCs or myeloid cells.

112. The composition of claim 111, wherein the myeloid cells are MPCs.

113. The composition of claim 109, wherein the pluripotent cells are ESCs or iPSCs.

114. The composition of any one of claims 82-108, wherein the cells are BLPCs, microglial progenitor cells, macrophages, or microglia.

115. The composition of claim 114, wherein the BLPCs are monocytes.

116. The composition of any one of claims 82-115, wherein, upon administration of the composition to a subject, expression of the TREM2 transgene is higher in myeloid cells in the subject than in lymphoid cells in the subject.

117. The composition of claim 116, wherein, upon administration of the composition to the subject, expression of the TREM2 transgene is higher in myeloid cells in the subject by at least 50% than in lymphoid cells in the subject; optionally, wherein, upon administration of the composition to the subject, expression of the TREM2 transgene is higher in myeloid cells in the subject by at least 60% than in lymphoid cells in the subject; optionally, wherein, upon administration of the composition to the subject, expression of the TREM2 transgene is higher in myeloid cells in the subject by at least 70% than in lymphoid cells in the subject; optionally, wherein, upon administration of the composition to the subject, expression of the TREM2 transgene is higher in myeloid cells in the subject by at least 80% than in lymphoid cells in the subject; optionally, wherein, upon administration of the composition to the subject, expression of the TREM2 transgene is higher in myeloid cells in the subject by at least 90% than in lymphoid cells in the subject; optionally, wherein, upon administration of the composition to the subject, expression of the TREM2 transgene is higher in myeloid cells in the subject by at least 95% than in lymphoid cells in the subject; optionally, wherein, upon administration of the composition to the subject, expression of the TREM2 transgene is higher in myeloid cells in the subject by at least 100%; optionally, wherein, upon administration of the composition to the subject, expression of the TREM2 transgene is higher in myeloid cells in the subject by at least 150%; optionally, wherein, upon administration of the composition to the subject, expression of the TREM2 transgene is higher in myeloid cells in the subject by at least 200%; optionally, wherein, upon administration of the composition to the subject, expression of the TREM2 transgene is higher in myeloid cells in the subject by at least 250%; optionally, wherein, upon administration of the composition to the subject, expression of the TREM2 transgene is higher in myeloid cells in the subject by at least 300%; optionally, wherein, upon administration of the composition to the subject, expression of the TREM2 transgene is higher in myeloid cells in the subject by at least 350%; optionally, wherein, upon administration of the composition to the subject, expression of the TREM2 transgene is higher in myeloid cells in the subject by at least 400%; optionally, wherein, upon administration of the composition to the subject, expression of the TREM2 transgene is higher in myeloid cells in the subject by at least 450%; optionally, wherein, upon administration of the composition to the subject, expression of the TREM2 transgene is higher in myeloid cells in the subject by at least 500%.

118. The composition of claim 116 or 117, wherein the lymphoid cells are B cells or T cells.

119. The composition of any one of claims 82-118, wherein the cells are transfected or transduced ex vivo to express the TREM2.

120. A pharmaceutical composition comprising the composition of any one of claims 82-119, wherein the pharmaceutical composition further comprises a pharmaceutically acceptable carrier, diluent, or excipient.

121. A kit comprising the composition of any one of claims 82-119, or the pharmaceutical composition of claim 120, and a package insert, wherein the package insert instructs a user of the kit to perform the method of any one of claims 1-81.
